(19) United States
(12) Patent Application Publication
(10) Pub. No.: US 2017/0266276 A1
(43) Pub. Date: Sep. 21, 2017

(54) Combination Therapy For Treatment of Cancer

(71) Applicant: OncoMed Pharmaceuticals, Inc., Redwood City, CA (US)

(72) Inventors: Austin L. GURNEY, San Francisco, CA (US); Wan-Ching YEN, Foster City, CA (US); Timothy Charles HOEY, HILLSBOROUGH, CA (US)

(21) Appl. No.: 15/531,876

(22) PCT Filed: Dec. 2, 2015
(86) PCT No.: PCT/US15/63480

§ 371 Date: May 31, 2017

Related U.S. Application Data
(60) Provisional application No. 62/086,435, filed on Dec. 2, 2014.
(60) Provisional application No. 62/210,545, filed on Aug. 27, 2015.

(51) Int. Cl. A61K 39/385; A61K 39/395; A61K 39/44; A61K 39/00

(52) U.S. Cl. CPC A61K 39/385; A61K 39/0011; A61K 39/39558; A61K 39/39591; A61K 39/44; C07K 2317/30; C07K 2317/32; C07K 2317/24; C07K 2317/22

(57) ABSTRACT

Described herein are combination therapies for the treatment of cancer and other diseases. In one aspect, the methods described herein for the treatment of cancer and other diseases comprise administering an RSPO-LGR pathway inhibitor in combination with a mitotic inhibitor.

### CROSS-REFERENCE TO RELATED APPLICATIONS

This application claims the priority benefit of U.S. Provisional Application No. 62/086,435, filed Dec. 2, 2014 and U.S. Provisional Application No. 62/210,545, filed Aug. 27, 2015, each of which is hereby incorporated by reference herein in its entirety.

### FIELD OF THE INVENTION

Described herein are combination therapies for the treatment of cancer and other diseases. In one aspect, the methods described herein for the treatment of cancer and other diseases comprise administering a RSPO-LGR pathway inhibitor in combination with a mitotic inhibitor.

### BACKGROUND OF THE INVENTION

The R-Spondin (RSPO) family of proteins is conserved among vertebrates and comprises four members, RSPO1, RSPO2, RSPO3, and RSPO4. These proteins have been referred to by a variety of names, including roof plate-specific spondins, hPWTSR (hRSPO3), THS2D (RSPO3), Cristin 1-4, and Futrin 1-4. The RSPOs are small secreted proteins that overall share approximately 40-60% sequence homology and domain organization. All RSPO proteins contain two furin-like cysteine-rich domains at the N-terminus followed by a thrombospondin domain and a basic charged C-terminal tail (Kim et al., 2006, Cell Cycle, 5:23-26).

Studies have shown that RSPO proteins have a role during vertebrate development (Kamata et al., 2004, Biochim. Biophys Acta, 1676:51-62) and in Xenopus myogenesis (Kazanskaya et al., 2004, Dev. Cell, 7:525-534). RSPO1 has also been shown to function as a potent mitogen for gastrointestinal epithelial cells (Kim et al., 2005, Science, 309:1256-1259). It has been reported that RSPO3 is prominently expressed in or close to endothelial cells and their cellular precursors in Xenopus and mouse. Furthermore, it has been suggested that RSPO3 can act as an angiogenic factor in embryogenesis (Kazanskaya et al., 2008, Development, 135:3655-3664).

Wnt ligands and R-spondin (RSPO) proteins have been shown to synergize to activate the canonical Wnt pathway. RSPO proteins are known to activate β-catenin signaling similar to Wnt signaling, however the relationship between RSPO proteins and Wnt signaling is still being investigated. It has been reported that RSPO proteins possess a positive modulatory activity on Wnt ligands (Nam et al., 2006, JBC 281:13247-57). This study also reported that RSPO proteins could function as Frizzled8 and LRP6 receptor ligands and induce β-catenin signaling (Nam et al., 2006, JBC 281:13247-57). Recent studies have identified an interaction between RSPO proteins and LGR (leucine-rich repeat containing, G protein-coupler receptor) proteins, such as LGR5 (U.S. Patent Publication Nos. 2009/0074782 and 2009/0191205), and these data present an alternative pathway for the activation of β-catenin signaling.

RSPO and LGR antagonists (e.g., anti-RSPO3 antibodies) that disrupt β-catenin signaling are a potential source of new therapeutic agents for cancer, as well as other β-catenin-associated diseases. See, e.g., U.S. Pat. No. 8,158,757, U.S. Pat. No. 8,540,989, U.S. Pat. No. 8,802,097, and U.S 20140017253.

Wnt pathway activation is associated with colorectal cancer. Approximately 5-10% of all colorectal cancers are hereditary with one of the main forms being familial adenomatous polyposis (FAP), an autosomal dominant disease in which about 80% of affected individuals contain a germline mutation in the adenomatous polyposis coli (APC) gene. Mutations have also been identified in other Wnt pathway components including Axin and β-catenin. Individual adenomas are clonal outgrowths of epithelial cells containing a second inactivated allele, and the large number of FAP adenomas inevitably results in the development of adenocarcinomas through additional mutations in oncogenes and/or tumor suppressor genes. Furthermore, activation of the Wnt signaling pathway, including loss-of-function mutations in APC and stabilizing mutations in β-catenin, can induce hyperplastic development and tumor growth in mouse models (Oshima et al., 1997, Cancer Res., 57:1644-9; Harada et al., 1999, EMBO J., 18:5931-42)

It is one of the objectives of the present invention to provide improved methods for cancer treatment, particularly strategically time-spaced (i.e., staggered) dosing regimens using a RSPO-LGR pathway inhibitor in combination with mitotic inhibitors.

### BRIEF SUMMARY OF THE INVENTION

The present invention relates to methods of treating cancer comprising administering to a subject a therapeutically effective amount of an RSPO-LGR pathway inhibitor, such as an anti-RSPO3 antibody or anti-LGR5 antibody. In certain embodiments, the methods further comprise administration of a mitotic inhibitor to the patient. In certain embodiments the RSPO-LGR pathway inhibitor is administered about 1 day, about 2 days, or about 3 days prior to the mitotic inhibitor. In some embodiments, the cancer is lung cancer. In certain other embodiments, the cancer is colorectal cancer, including without limitation colorectal cancer comprising an inactivating mutation in the adenomatous polyposis coli (APC) gene or an activating mutation in the β-catenin gene.

The present invention further relates to a method of treating cancer comprising administering to a subject a therapeutically effective amount of a RSPO-LGR pathway inhibitor and a therapeutically effective amount of a mitotic inhibitor, wherein the RSPO-LGR inhibitor and the mitotic inhibitor are administered using a staggered dosing schedule and the RSPO-LGR inhibitor is administered first; and wherein the RSPO-LGR pathway inhibitor is: (a) an antibody that specifically binds at least one human RSPO protein, (b) an antibody that specifically binds at least one human LGR protein, or (c) a soluble receptor comprising an extracellular domain of a human LGR protein capable of binding at least one human RSPO protein.

The present invention also relates to a method of treating cancer comprising administering to a subject a therapeutically effective amount of an antibody that specifically binds at least one human RSPO protein and a therapeutically effective amount of a mitotic inhibitor, wherein the antibody and the mitotic inhibitor are administered using a staggered dosing schedule and the antibody is administered first.

The present invention also relates to a method of treating cancer comprising administering to a subject a therapeutically effective amount of an antibody that specifically binds human RSPO3 and a therapeutically effective amount of a mitotic inhibitor, wherein the antibody and the mitotic inhibitor are administered using a staggered dosing schedule and the antibody is administered first. In one embodiment the mitotic inhibitor is administered about 1, 2, 3, 4, 5, or 6 days after the RSPO-LGR pathway inhibitor or antibody is administered.

The present invention also relates to a method of treating cancer in a subject comprising administering to the subject a therapeutically effective amount of a RSPO-LGR pathway inhibitor, wherein the RSPO-LGR pathway inhibitor is: (a) an antibody that specifically binds at least one human RSPO protein, (b) an antibody that specifically binds at least one human LGR protein, or (c) a soluble receptor comprising an extracellular domain of a human LGR protein capable of binding at least one human RSPO protein, and wherein the subject is scheduled to receive a therapeutically effective amount of a mitotic inhibitor about 1, 2, 3, 4, 5, or 6 days after the administration of the RSPO-LGR pathway inhibitor.

The present invention also relates to a method of treating cancer in a subject comprising administering to the subject a therapeutically effective amount of an antibody that specifically binds at least one human RSPO protein, wherein the subject is scheduled to receive a therapeutically effective amount of a mitotic inhibitor about 1, 2, 3, 4, 5, or 6 days after the administration of the antibody.

The present invention also relates to a method of treating cancer in a subject comprising administering to the subject a therapeutically effective amount of an antibody that specifically binds human RSPO3, wherein the subject is scheduled to receive a therapeutically effective amount of a mitotic inhibitor about 1, 2, 3, 4, 5, or 6 days after the administration of the antibody.

The present invention also relates to a method of increasing the efficacy of a mitotic inhibitor in treating cancer in a subject comprising administering to the subject a mitotic inhibitor about 1, 2, 3, 4, 5, or 6 days after a RSPO-LGR pathway inhibitor has been administered, wherein the RSPO-LGR pathway inhibitor is: (a) an antibody that specifically binds at least one human RSPO protein, (b) an antibody that specifically binds at least one human LGR protein, or (c) a soluble receptor comprising an extracellular domain of a human LGR protein capable of binding at least one human RSPO protein.

The present invention also relates to a method of increasing the efficacy of a mitotic inhibitor in treating cancer in a subject comprising administering to the subject a mitotic inhibitor about 1, 2, 3, 4, 5, or 6 days after an antibody that specifically binds at least one human RSPO protein has been administered.

The present invention also relates to a method of increasing the efficacy of a mitotic inhibitor in treating cancer in a subject comprising administering to the subject a mitotic inhibitor about 1, 2, 3, 4, 5, or 6 days after an antibody that specifically binds human RSPO3 has been administered.

The present invention also relates to a method of increasing the efficacy of a mitotic inhibitor in treating cancer in a subject comprising: (a) administering to the subject an RSPO-LGR pathway inhibitor; and (b) administering to the subject a mitotic inhibitor about 1, 2, 3, 4, 5, or 6 days after the RSPO-LGR pathway inhibitor has been administered, wherein the RSPO-LGR pathway inhibitor is: (i) an antibody that specifically binds at least one human RSPO protein, (ii) an antibody that specifically binds at least one human LGR protein, or (iii) a soluble receptor comprising an extracellular domain of a human LGR protein capable of binding at least one human RSPO protein.

The present invention also relates to a method of increasing the efficacy of a mitotic inhibitor in treating cancer in a subject comprising: (a) administering to the subject an antibody that specifically binds at least one human RSPO protein; and (b) administering to the subject a mitotic inhibitor about 1, 2, 3, 4, 5, or 6 days after the antibody has been administered.

The present invention also relates to a method of increasing the efficacy of a mitotic inhibitor in treating cancer in a subject comprising: (a) administering to the subject an antibody that specifically binds human RSPO3; and (b) administering to the subject a mitotic inhibitor about 1, 2, 3, 4, 5, or 6 days after the antibody has been administered.

In some embodiments of the aforementioned methods, the mitotic inhibitor is administered about 1 day after administration of the RSPO-LGR pathway inhibitor or antibody. In one embodiment, the mitotic inhibitor is administered about 2 days after administration of the RSPO-LGR pathway inhibitor or antibody. In another embodiment, it is administered about 3 days after administration of the RSPO-LGR pathway inhibitor or antibody.

In some embodiments of the aforementioned methods, the RSPO-LGR pathway inhibitor or antibody and the mitotic inhibitor act synergistically.

In some embodiments of the present invention, the RSPO-LGR pathway inhibitor or antibody is administered once a week. In some embodiments of the present invention, the RSPO-LGR pathway inhibitor or antibody is administered once every 2 weeks. In some embodiments of the present invention, the RSPO-LGR pathway inhibitor or antibody is administered once every 3 weeks.

In some embodiments of the present invention, the RSPO-LGR pathway inhibitor or antibody is administered about once every 2 weeks and the mitotic inhibitor is administered about once a week. In another embodiment, the mitotic inhibitor is administered about once every 2 weeks, about once every 3 weeks, or once every week for 3 weeks out of a 4 week (28 day) cycle. In another embodiment, the RSPO-LGR pathway inhibitor or antibody is administered for 2, 3, 4, 5, 6, 7, 8, or more cycles. In another embodiment, the mitotic inhibitor is administered for 2, 3, 4, 5, 6, 7, 8, or more cycles.

In some embodiments of the present invention, the RSPO-LGR pathway inhibitor or antibody is administered about once every 3 weeks and the mitotic inhibitor is administered about once a week. In another embodiment, the RSPO-LGR pathway inhibitor or antibody is administered about once every 4 weeks. In another embodiment, the mitotic inhibitor is administered about once a week, about once every 2 weeks, or about once every 3 weeks. In another embodiment, the RSPO-LGR pathway inhibitor or antibody is administered about once every 4 weeks and the mitotic inhibitor is administered about once a week. In another embodiment, the RSPO-LGR pathway inhibitor or antibody is administered for 2, 3, 4, 5, 6, 7, 8, or more cycles. In another embodiment, the mitotic inhibitor is administered for 2, 3, 4, 5, 6, 7, 8, or more cycles.

In some embodiments of the present invention, the RSPO-LGR pathway inhibitor is administered to the subject at a dosage of about 2 mg/kg to about 20 mg/kg. In some embodiments, the RSPO-LGR pathway inhibitor or antibody is administered to the subject at a dosage of about 2 mg/kg to about 10 mg/kg. In some embodiments, the RSPO-LGR pathway inhibitor or antibody is administered to the subject at a dosage of about 2.5 mg/kg to about 10 mg/kg. In some embodiments, the RSPO-LGR pathway inhibitor or antibody is administered to the subject at a dosage of about 5 mg/kg to about 20 mg/kg.

In another embodiment, the RSPO-LGR pathway inhibitor or antibody is administered at a dosage of about 2 mg/kg to about 20 mg/kg once a week. In another embodiment, the RSPO-LGR pathway inhibitor or antibody is administered at a dosage of about 2 mg/kg to about 20 mg/kg once every two weeks. In another embodiment, the RSPO-LGR pathway inhibitor or antibody is administered at a dosage of about 2 mg/kg to about 20 mg/kg once every three weeks. In another embodiment, the RSPO-LGR pathway inhibitor or antibody is administered at a dosage of about 2 mg/kg to about 20 mg/kg once every four weeks. In another embodiment, the RSPO-LGR pathway inhibitor or antibody is administered at a dosage of about 2 mg/kg to about 5 mg/kg every three weeks. In another embodiment, the RSPO-LGR pathway inhibitor or antibody is administered at a dosage of about 3 mg/kg to about 7.5 mg/kg every four weeks.

In some embodiments of the present invention, the RSPO-LGR pathway inhibitor of the invention is an antibody that specifically binds at least one human RSPO protein. In one embodiment, the antibody specifically binds at least one human RSPO protein selected from the group consisting of: RSPO1, RSPO2, and RSPO3. In another embodiment, the antibody specifically binds at least human RSPO1. In another embodiment, the antibody comprises: (a) a heavy chain CDR1 comprising TGYTMH (SEQ ID NO:5), a heavy chain CDR2 comprising GINPNNGGTTYNQNFKG (SEQ ID NO:6), and a heavy chain CDR3 comprising KEFSDGYYFFAY (SEQ ID NO:7); and (b) a light chain CDR1 comprising KASQDVIFAVA (SEQ ID NO:8), a light chain CDR2 comprising WASTRHT (SEQ ID NO:9), and a light chain CDR3 comprising QQHYSTPW (SEQ ID NO:10). In another embodiment, the antibody comprises a heavy chain variable region comprising SEQ ID NO:11 or SEQ ID NO:44, and a light chain variable region comprising SEQ ID NO:12 or SEQ ID NO:45. In another embodiment, the antibody comprises a heavy chain variable region comprising SEQ ID NO:11 and a light chain variable region comprising SEQ ID NO:12. In another embodiment, the antibody comprises a heavy chain variable region comprising SEQ ID NO:44 and a light chain variable region comprising SEQ ID NO:45.

In some embodiments of the present invention, the antibody of the invention specifically binds at least human RSPO2. In another embodiment, the antibody comprises: (a) a heavy chain CDR1 comprising SSYAMS (SEQ ID NO:17), a heavy chain CDR2 comprising SISSGGSTYYPDSVKG (SEQ ID NO:18), and a heavy chain CDR3 comprising RGGDPGVYNGDYEDAMDY (SEQ ID NO:19); and (b) a light chain CDR1 comprising KASQDVSSAVA (SEQ ID NO:20), a light chain CDR2 comprising WASTRHT (SEQ ID NO:21), and a light chain CDR3 comprising QQHYSTP (SEQ ID NO:22). In another embodiment, the antibody comprises a heavy chain variable region comprising SEQ ID NO:23 and a light chain variable region comprising SEQ ID NO:24.

In some embodiments of the present invention, the antibody of the invention specifically binds at least human RSPO3. In another embodiment, the antibody comprises: (a) a heavy chain CDR1 comprising DYSIH (SEQ ID NO:29), a heavy chain CDR2 comprising YIYPSNGDSGYNQKFK (SEQ ID NO:30), and a heavy chain CDR3 comprising TYFANNFD (SEQ ID NO:31) or ATYFANNTDY (SEQ ID NO:32); and (b) a light chain CDR1 comprising KASQSVDYDGDSYMN (SEQ ID NO:33), a light chain CDR2 comprising AASNLES (SEQ ID NO:34) or AAS (SEQ ID NO:35), and a light chain CDR3 comprising QQSNEDPLT (SEQ ID NO:36) or QQSNEDPLTF (SEQ ID NO:37). In another embodiment, the antibody comprises: (a) a heavy chain CDR1 comprising DYSIH (SEQ ID NO:29), a heavy chain CDR2 comprising YIYPSNGDSGYNQKFK (SEQ ID NO:30), and a heavy chain CDR3 comprising TYFANNFD (SEQ ID NO:31); and (b) a light chain CDR1 comprising KASQSVDYDGDSYMN (SEQ ID NO:33), a light chain CDR2 comprising AASNLES (SEQ ID NO:34), and a light chain CDR3 comprising QQSNEDPLT (SEQ ID NO:36). In another embodiment, the antibody comprises a heavy chain variable region comprising SEQ ID NO:38 and a light chain variable region comprising SEQ ID NO:39.

In some embodiments of the present invention, the RSPO-LGR pathway inhibitor is an antibody that specifically binds at least one human LGR protein. In another embodiment, the antibody specifically binds at least one human LGR protein selected from the group consisting of: LGR4, LGR5, and LGR6. In another embodiment, the antibody specifically binds at least human LGR5. In another embodiment, the antibody comprises: (a) the heavy chain CDR1, CDR2, and CDR3 sequences of the monoclonal antibody produced by the 88M1 hybridoma having the ATCC deposit number PTA-9342; and (b) the light chain CDR1, CDR2, and CDR3 sequences of the monoclonal antibody produced by the 88M1 hybridoma having the ATCC deposit number PTA-9342. In another embodiment, the antibody comprises the heavy chain variable region and light chain variable region of the monoclonal antibody produced by the 88M1 hybridoma having the ATCC deposit number PTA-9342.

In some embodiments of the aforementioned methods, the antibody is a monoclonal antibody, a recombinant antibody, a chimeric antibody, a humanized antibody, a human antibody, or an antibody fragment comprising an antigen-binding site. In another embodiment, the antibody is a monospecific antibody or a bispecific antibody. In another embodiment, the antibody is an IgG1 antibody, an IgG2 antibody, or an IgG4 antibody.

In one embodiment of the present invention, the RSPO-LGR pathway inhibitor is OMP-131R010.

In some embodiments of the present invention, the RSPO-LGR pathway inhibitor is a soluble receptor comprising an extracellular domain of a human LGR protein or a fragment thereof, wherein the extracellular domain is capable of binding a human RSPO protein. In another embodiment, the human LGR protein is LGR5. In another embodiment, the extracellular domain of a human LGR protein comprises amino acids 22-564 of human LGR5 (SEQ ID NO: 56). In another embodiment, the soluble receptor comprises a non-LGR polypeptide. In another embodiment, the non-LGR polypeptide is directly linked to the extracellular domain of the human LGR protein. In another embodiment, the non-LGR polypeptide is connected to the extracellular domain of the human LGR protein by a linker. In another embodiment, the non-LGR polypeptide comprises a human Fc region. In another embodiment, the non-LGR polypeptide comprises SEQ ID NO:58, SEQ ID NO:59, SEQ ID NO:60, SEQ ID NO:61, or SEQ ID NO:62.

In some embodiments of the aforementioned methods, the mitotic inhibitor of the invention is a taxane, a vinca alkaloid, an epothilone, or eribulin mesylate. In another embodiment, the mitotic inhibitor is a taxane selected from the group consisting of paclitaxel, docetaxel, and derivatives thereof. In another embodiment, the mitotic inhibitor is paclitaxel or nab-paclitaxel. In another embodiment, the mitotic inhibitor is docetaxel. In another embodiment, the mitotic inhibitor is a vinca alkaloid selected from the group consisting of vinblastine, vincristine, vinorelbine, and derivatives thereof.

In some embodiments of the aforementioned methods, the cancer of the invention is colorectal cancer, breast cancer, ovarian cancer, lung cancer, or pancreatic cancer. In another embodiment, the cancer is colorectal cancer. In some embodiments, Wnt signaling is activated in the colorectal cancer (e.g., by an inactivating mutation in the APC gene or an activating mutation in the β-catenin gene). In certain embodiments, the colorectal cancer is third-line colorectal cancer. In some embodiments, the colorectal cancer is resistant to treatment with chemotherapy comprising 5-fluorouracil, irinotecan, and/or oxaliplatin.

In some embodiments, the aforementioned methods of the invention further comprise administering at least one additional therapeutic agent. In another embodiment, the additional therapeutic agent is a chemotherapeutic agent.

The present invention also relates to a method of treating cancer comprising administering to a subject a therapeutically effective amount of OMP-131R010 and a therapeutically effective amount of a taxane selected from the group consisting of paclitaxel, nab-paclitaxel, and docetaxel, wherein the taxane is administered about 1, 2, 3, 4, 5, or 6 days after OMP-131R010 is administered. In one embodiment of the invention, OMP-131R010 is administered about once every 3 weeks. In another embodiment, OMP-131R010 is administered about once every 4 weeks. In another embodiment, the taxane is administered about once a week. In another embodiment, the taxane is administered about once every two weeks. In another embodiment, the taxane is administered about once every three weeks.

In some embodiments of the aforementioned methods, an additional therapeutic agent is also administered. In one embodiment, the additional therapeutic agent is a chemotherapeutic agent.

In some embodiments of the aforementioned methods, the cancer is colorectal cancer, breast cancer, ovarian cancer, lung cancer, or pancreatic cancer. In some embodiments, the cancer comprises a RSPO gene fusion. In some embodiments, the cancer comprises a RSPO2 gene fusion. In some embodiments, the cancer comprises a RSPO3 gene fusion. In one embodiment of the invention, the cancer is colorectal cancer. In another embodiment, the colorectal cancer comprises an inactivating mutation in the adenomatous polyposis coli (APC) gene. In another embodiment, the colorectal cancer does not comprise an inactivating mutation in the APC gene. In another embodiment, the colorectal cancer comprises a wild-type APC gene. In another embodiment, the colorectal cancer comprises an activating mutation in the β-catenin gene. In another embodiment, the colorectal cancer does not comprise an activating mutation in the β-catenin gene. In another embodiment, the colorectal cancer comprises a RSPO gene fusion. In another embodiment, the RSPO gene fusion is a RSPO2 gene fusion. In another embodiment, the RSPO gene fusion is a RSPO3 gene fusion.

In some embodiments of the present invention, the presence of an inactivating mutation in the APC gene of the cancer is determined. In another embodiment, the presence of an activating mutation in the β-catenin gene of the tumor or cancer is determined. In another embodiment, the presence of a RSPO gene fusion in the tumor or cancer is determined. In another embodiment, the RSPO gene fusion is a RSPO2 gene fusion. In another embodiment, the RSPO gene fusion is a RSPO3 gene fusion. In another embodiment, the presence of the RSPO gene fusion is determined by a PCR-based assay, microarray analysis, or nucleotide sequencing.

In some embodiments of the present invention, the cancer expresses high RSPO1, RSPO2, RSPO3, and/or RSPO4 levels compared to a pre-determined level of expression of RSPO1, RSPO2, RSPO3, and/or RSPO4, respectively. In another embodiment, the pre-determined RSPO1, RSPO2, RSPO3, or RSPO4 expression level is the expression level of RSPO1, RSPO2, RSPO3, or RSPO4 in a tumor or a group of tumors of the same tissue type. In another embodiment, the pre-determined RSPO1, RSPO2, RSPO3, or RSPO4 expression level is the expression level of RSPO1, RSPO2, RSPO3, or RSPO4 in normal tissue of the same tissue type.

In one embodiment of the invention, the expression level of one or more of RSPO1, RSPO2, RSPO3, and RSPO4 in the cancer is also determined. In another embodiment, the expression level of one or more of RSPO1, RSPO2, RSPO3, and RSPO4 is determined by a PCR-based assay, microarray analysis, or nucleotide sequencing.

Where aspects or embodiments are described in terms of a Markush group or other grouping alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTION OF THE DRAWINGS/FIGURES

FIGS. 1A and 1B. Inhibition of ovarian tumor growth in vivo by a RSPO-LGR pathway inhibitor in combination with a chemotherapeutic agent. OMP-OV19 ovarian tumor cells were injected subcutaneously into NOD/SCID mice. FIG. 1A. Mice were treated with control antibody (--), paclitaxel (-▪-), a combination of paclitaxel plus anti-RSPO3 antibody 131R010 (also referred to as OMP-131R10) administered the same day (-▴-), or a combination of paclitaxel plus 131R010, wherein 131R010 was administered 2 days prior to paclitaxel (-▾-). Antibodies were dosed at 25 mg/kg and administered every other week. Paclitaxel was dosed at 20 mg/kg and administered every other week. Tumor volumes were measured on the indicated days post-treatment and shown as the mean±SEM. FIG. 1B. Tumor volumes of individual animals in the two combination treatment groups on Day 61.

FIG. 2. Inhibition of lung tumor growth in vivo by a RSPO-LGR pathway inhibitor in combination with a chemotherapeutic agent. OMP-LU77 lung tumor cells were injected subcutaneously into NOD/SCID mice. Mice were treated with control antibody (-▪-), paclitaxel (-▴-), a combination of paclitaxel and anti-RSPO3 antibody 131R010 (also referred to as OMP-131R10) administered the same day (-▾-), or a combination of paclitaxel plus anti-RSPO3 antibody 131R010, wherein 131R010 was administered 2 days prior to paclitaxel (--). Antibodies were dosed every three weeks at 25 mg/kg. Taxol was dosed every three weeks at 20 mg/kg. Tumor volumes were measured on the indicated days post-treatment.

FIGS. 3A and 3B. Inhibition of colorectal tumor growth in vivo by a RSPO-LGR pathway inhibitor in combination with chemotherapeutic agents. OMP-C8 colorectal tumor cells were injected subcutaneously into NOD/SCID mice. FIG. 3A. Mice were treated with control antibody (-▪-), nab-paclitaxel (ABRAXANE) (-Δ-), a combination of nab-paclitaxel and anti-RSPO3 antibody 131R010 administered the same day (--), or a combination of nab-paclitaxel plus anti-RSPO3 antibody 131R010, wherein 131R010 was administered 2 days prior to nab-paclitaxel (-▾-). Antibodies were dosed every other week at 25 mg/kg. Nab-paclitaxel was dosed once a week at 30 mg/kg. Tumor volumes were measured on the indicated days post-treatment. FIG. 3B. Mice were treated with control antibody (-▪-), 5-FU and irinotecan (-◯-), a combination of 5-FU, irinotecan, and anti-RSPO3 antibody 131R010 administered the same day (-▴-), or a combination of 5-FU and irinotecan plus anti-RSPO3 antibody 131R010, wherein 131R010 was administered 2 days prior to 5-FU and irinotecan (-⋄-). Antibodies were dosed every other week at 25 mg/kg. 5-FU and irinotecan were dosed at 50 mg/kg and 5 mg/kg, respectively, once a week. Tumor volumes were measured on the indicated days post-treatment.

### DETAILED DESCRIPTION OF THE INVENTION

Described herein are methods of inhibiting tumor growth, methods of reducing tumor size, and methods of treating cancer. The methods provided herein comprise administering to a subject a therapeutically effective amount of a RSPO-LGR pathway inhibitor in combination with a therapeutically effective amount of a mitotic inhibitor using a staggered dosing schedule. In some embodiments, the RSPO-LGR pathway inhibitor is an antibody. In some embodiments, the RSPO-LGR pathway inhibitor is an antibody that specifically binds at least one RSPO protein. In some embodiments, the RSPO-LGR pathway inhibitor is an antibody that specifically binds at least one LGR protein. In some embodiments, the RSPO-LGR pathway inhibitor is a soluble receptor. In some embodiments, the RSPO-LGR pathway inhibitor is a soluble receptor comprising an extracellular domain of a LGR protein or a fragment thereof. In some embodiments, the mitotic inhibitor is a taxane, a vinca alkaloid, an epothilone, or eribulin mesylate.

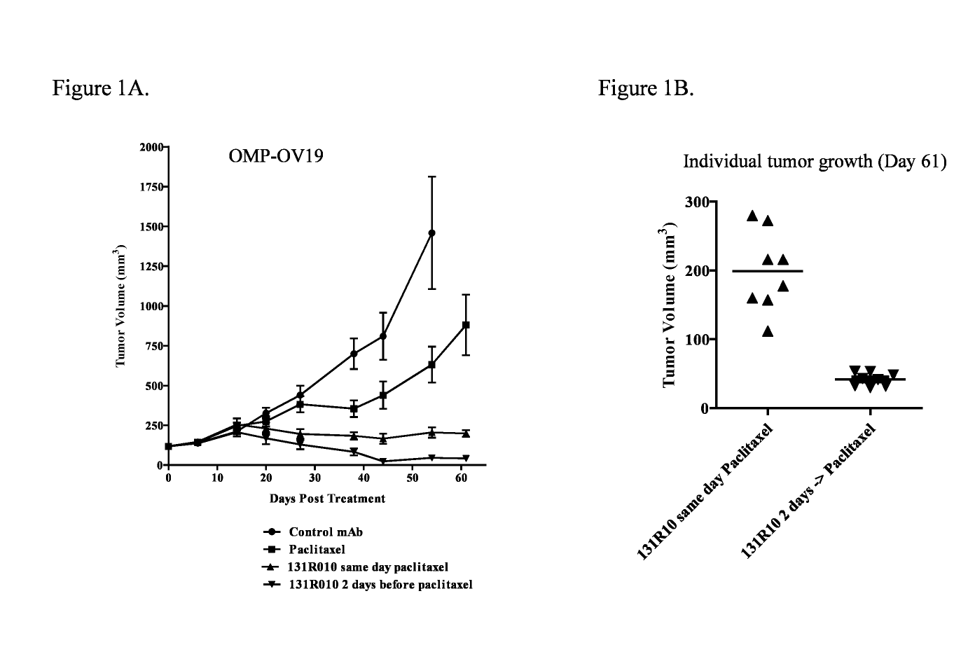

Treatment with a combination of the RSPO-LGR pathway inhibitor anti-RSPO3 antibody OMP-131R010 (also referred to as OMP-131R10) and a taxane was effective at inhibiting tumor growth in several xenograft models. Surprisingly, the order of delivering the anti-RSPO3 antibody and the taxane affected the efficacy of the drug combination. Administration of the RSPO-LGR pathway inhibitor anti-RSPO3 antibody OMP-131R010 prior to administration of a taxane (staggered or sequential manner of dosing) was better at inhibiting tumor growth in the xenograft models than co-administration of OMP-131R010 and taxane (e.g., Examples 1-3; FIGS. 1, 2, and 3A). Indeed, staggered administration of OMP-131R010 and taxane not only inhibited tumor growth, but actually decreased tumor size over the course of the treatment.

### I. Definitions

To facilitate an understanding of the detailed description, a number of terms and phrases are defined below.

The terms “antagonist” and “antagonistic” as used herein refer to any molecule that partially or fully blocks, inhibits, reduces, or neutralizes a biological activity of a target and/or signaling pathway (e.g., the RSPO-LGR pathway). The term “antagonist” is used herein to include any molecule that partially or fully blocks, inhibits, reduces, or neutralizes the activity of a protein (e.g., a RSPO protein or an LGR protein). Suitable antagonist molecules specifically include, but are not limited to, antagonist antibodies, antibody fragments, soluble receptors, or small molecules.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen-binding site within the variable region of the immunoglobulin molecule. As used herein, the term encompasses intact polyclonal antibodies, intact monoclonal antibodies, antibody fragments comprising an antigen-binding site (such as Fab, Fab′, F(ab′)2, and Fv fragments), single chain Fv (scFv) antibodies, multispecific antibodies such as bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” as used herein refers to a portion of an intact antibody and generally includes the antigenic determining variable region or antigen-binding site of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises at least one antigen-binding site or epitope-binding site.

The term “variable region” of an antibody as used herein refers to the variable region of the antibody light chain, or the variable region of the antibody heavy chain, either alone or in combination. The variable region of the heavy or light chain generally consists of four framework regions connected by three complementarity determining regions (CDRs), also known as “hypervariable regions”. The CDRs in each chain are held together in close proximity by the framework regions and, with the CDRs from the other chain, contribute to the formation of the antigen-binding site of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.), and (2) an approach based on crystallographic studies of antigen-antibody complexes (Al-Lazikani et al., 1997, J. Mol. Biol., 273:927-948). In addition, combinations of these two approaches are sometimes used in the art to determine CDRs.

The term “monoclonal antibody” as used herein refers to a homogenous antibody population involved in the highly specific recognition and binding of a single antigenic determinant or epitope. This is in contrast to polyclonal antibodies that typically include a mixture of different antibodies directed against different antigenic determinants. The term “monoclonal antibody” encompasses both intact and full-length antibodies as well as antibody fragments (e.g., Fab, Fab′, F(ab′)2, Fv), single chain (scFv) antibodies, fusion proteins comprising an antibody portion, and any other modified immunoglobulin molecule comprising at least one antigen-binding site. Furthermore, “monoclonal antibody” refers to such antibodies made by any number of techniques, including but not limited to, hybridoma production, phage selection, recombinant expression, and transgenic animals.

The term “humanized antibody” as used herein refers to antibodies that are specific immunoglobulin chains, chimeric immunoglobulins, or fragments thereof that contain minimal non-human sequences. Typically, humanized antibodies are human immunoglobulins in which amino acid residues of the CDRs are replaced by amino acid residues from the CDRs of a non-human species (e.g., mouse, rat, rabbit, or hamster) that have the desired specificity, affinity, and/or binding capability.

The term “human antibody” as used herein refers to an antibody produced by a human or an antibody having an amino acid sequence corresponding to an antibody produced by a human made using any of the techniques known in the art.

The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and/or binding capability, while the constant regions are homologous to the sequences in antibodies derived from another species (usually human).

The term “affinity-matured antibody” as used herein refers to an antibody with one or more alterations in one or more CDRs that result in an improvement in the affinity of the antibody for antigen, compared to a parent antibody that does not possess those alterations(s). Preferred affinity-matured antibodies will have nanomolar or even picomolar affinities for the target antigen. Affinity-matured antibodies are produced by procedures known in the art including heavy chain and light chain variable region shuffling, random mutagenesis of CDR and/or framework residues, or site-directed mutagenesis of CDR and/or framework residues.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and non-contiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, or 8-10 amino acids in a unique spatial conformation.

The terms “selectively binds” or “specifically binds” as used herein mean that a binding agent or an antibody reacts or associates more frequently, more rapidly, with greater duration, with greater affinity, or with some combination of the above to the epitope, protein, or target molecule than with alternative substances, including unrelated or related proteins. In certain embodiments “specifically binds” means, for instance, that an antibody binds a target with a K_{D} of about 0.1 mM or less, but more usually less than about 1 μM. In certain embodiments, “specifically binds” means that an antibody binds a target with a K_{D} of at least about 0.1 μM or less, at least about 0.01 μM or less, or at least about 1 nM or less. Because of the sequence identity between homologous proteins in different species, specific binding can include an antibody that recognizes a protein in more than one species (e.g., human RSPO protein and mouse RSPO protein). Likewise, because of homology within certain regions of polypeptide sequences of different proteins, specific binding can include an antibody (or other polypeptide or binding agent) that recognizes more than one protein (e.g., human RSPO1 and human RSPO3). It is understood that, in certain embodiments, an antibody or binding agent that specifically binds a first target can or cannot specifically bind a second target. As such, “specific binding” does not necessarily require (although it can include) exclusive binding, i.e. binding to a single target. Thus, an antibody can, in certain embodiments, specifically bind more than one target. In certain embodiments, multiple targets can be bound by the same antigen-binding site on the antibody. For example, an antibody can, in certain instances, comprise two identical antigen-binding sites, each of which specifically binds the same epitope on two or more proteins (e.g., RSPO1 and RSPO3). In certain alternative embodiments, an antibody can be bispecific and comprise at least two antigen-binding sites with differing specificities. By way of non-limiting example, a bispecific antibody can comprise one antigen-binding site that recognizes an epitope on one protein (e.g., a human RSPO protein) and further comprise a second, different antigen-binding site that recognizes a different epitope on a second protein. Generally, but not necessarily, reference to binding means specific binding.

The term “soluble receptor” as used herein refers to an extracellular fragment (or a portion thereof) of a receptor protein preceding the first transmembrane domain of the receptor that can be secreted from a cell in soluble form.

The term “LGR soluble receptor” as used herein refers to an extracellular fragment of an LGR receptor protein (e.g., LGR5) preceding the first transmembrane domain of the receptor that can be secreted from a cell in soluble form. LGR soluble receptors comprising the entire extracellular domain (ECD) as well as smaller fragments of the ECD are encompassed by the term. In certain embodiments, the extracellular domain comprises amino acids 22-564 of human LGR5 (SEQ ID NO:56). In certain embodiments, the extracellular fragment is capable of binding at least one human RSPO protein.

The terms “polypeptide” and “peptide” and “protein” are used interchangeably herein and refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides used in the methods described herein can be based upon antibodies, in certain embodiments, the polypeptides can occur as single chains or associated chains.

The term “amino acid” as used herein refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. The phrase “amino acid analog” refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. The phrase “amino acid mimetic” refers to chemical compounds that have a structure that is different from the general chemical structure of an amino acid, but that function similarly to a naturally occurring amino acid.

The terms “polynucleotide” and “nucleic acid” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST and BLAST variations, ALIGN and ALIGN variations, Megalign, BestFit, GCG Wisconsin Package, etc. In some embodiments, two nucleic acids or polypeptides are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 nucleotides or residues, at least about 60-80 nucleotides or residues in length or any integral value therebetween. In some embodiments, identity exists over a longer region than 60-80 nucleotides or residues, such as at least about 80-100 nucleotides or residues, and in some embodiments the sequences are substantially identical over the full length of the sequences being compared, such as the coding region of a nucleotide sequence.

The term “conservative amino acid substitution” as used herein refers to a substitution in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Preferably, conservative substitutions in the sequences of the polypeptides and antibodies do not abrogate the binding of the polypeptide or antibody containing the amino acid sequence to the antigen(s). Methods of identifying amino acid conservative substitutions which do not eliminate antigen binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

As used herein, a polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The terms “cancer” and “cancerous” as used herein refer to or describe the physiological condition in mammals in which a population of cells is characterized by unregulated cell growth. Examples of cancer include, but are not limited to, carcinoma, blastoma, sarcoma, and hematologic cancers such as lymphoma and leukemia.

The terms “proliferative disorder” and “proliferative disease” as used herein refer to disorders associated with abnormal cell proliferation such as cancer.

The terms “tumor” and “neoplasm” as used herein refer to any mass of tissue that results from excessive cell growth or proliferation, either benign (non-cancerous) or malignant (cancerous), including pre-cancerous lesions.

The term “metastasis” as used herein refers to the process by which a cancer spreads or transfers from the site of origin to other regions of the body with the development of a similar cancerous lesion at the new location. A “metastatic” or “metastasizing” cell is generally one that loses adhesive contacts with neighboring cells and migrates from the primary site of disease to invade neighboring body structures.

The terms “cancer stem cell” and “CSC” and “tumor stem cell” and “tumor initiating cell” are used interchangeably herein and refer to cells from a cancer or tumor that: (1) have extensive proliferative capacity; (2) are capable of asymmetric cell division to generate one or more types of differentiated cell progeny wherein the differentiated cells have reduced proliferative or developmental potential; and (3) are capable of symmetric cell divisions for self-renewal or self-maintenance. These properties confer on the cancer stem cells the ability to form or establish a tumor or cancer upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” as used herein refer to the total population of cells derived from a cancer or tumor or pre-cancerous lesion, including both non-tumorigenic cells, which comprise the bulk of the cancer cell population, and tumorigenic cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a sample of cells from a tumor to form palpable tumors upon serial transplantation into immunocompromised hosts (e.g., mice).

The term “subject” as used herein refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The term “pharmaceutically acceptable” refers to an agent, compound, molecule, etc. approved or approvable by a regulatory agency of the Federal or a state government or listed in the U.S. Pharmacopeia or other generally recognized pharmacopeia for use in animals, including humans.

The phrases “pharmaceutically acceptable excipient, carrier or adjuvant” and “acceptable pharmaceutical carrier” refer to an excipient, carrier, or adjuvant that can be administered to a subject, together with a therapeutic agent, and which does not destroy the pharmacological activity thereof and is nontoxic when administered in doses sufficient to deliver a therapeutic effect. In general, those of skill in the art and the FDA consider a pharmaceutically acceptable excipient, carrier, or adjuvant to be an inactive ingredient of any formulation or pharmaceutical composition.

The terms “effective amount” and “therapeutically effective amount” and “therapeutic effect” as used herein refer to an amount of a binding agent, an antibody, a polypeptide, a polynucleotide, a small molecule, or other therapeutic agent effective to “treat” a disease or disorder in a subject or mammal. In the case of cancer, the therapeutically effective amount of an agent (e.g., an antibody) has a therapeutic effect and as such can reduce the number of cancer cells; decrease tumorigenicity, tumorigenic frequency, or tumorigenic capacity; reduce the number or frequency of cancer stem cells; reduce tumor size; reduce the cancer cell population; inhibit and/or stop cancer cell infiltration into peripheral organs including, for example, the spread of cancer into soft tissue and bone; inhibit and stop tumor or cancer cell metastasis; inhibit and/or stop tumor or cancer cell growth; relieve to some extent one or more of the symptoms associated with the cancer; reduce morbidity and mortality; improve quality of life; or a combination of such effects. To the extent the agent prevents growth and/or kills existing cancer cells, it can be referred to as cytostatic and/or cytotoxic.

The terms “treating” and “treatment” and “to treat” and “alleviating” and “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus, those in need of treatment include those who already have a disorder; those prone to have a disorder; and those in whom a disorder is to be prevented. In some embodiments, a subject is successfully “treated” according to the methods described herein if the patient shows one or more of the following: a reduction in the number of or complete absence of cancer cells; a reduction in tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

As used in the present disclosure and claims, the singular forms “a”, “an”, and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Methods of Use and Pharmaceutical Compositions

RSPO-LGR pathway inhibitors (e.g., RSPO-binding agents and LGR-binding agents) in combination with mitotic inhibitors are useful in a variety of applications including, but not limited to, therapeutic treatment methods, such as the treatment of cancer, particularly when used in a staggered or sequential dosing regimen. In certain embodiments, the combination of a RSPO-LGR pathway inhibitor and a mitotic inhibitor is useful in methods of inhibiting β-catenin signaling, inhibiting mitosis, inhibiting tumor growth, reducing tumor size, inducing tumor cell differentiation, inducing apoptosis, inducing tumor cell death, increasing tumor cell differentiation, increasing apoptosis, increasing tumor cell death, reducing tumor volume, reducing cancer stem cell frequency, and/or reducing the tumorigenicity of a tumor, particularly when used in a staggered or sequential dosing regimen. The methods of use can be in vitro, ex vivo, or in vivo methods.

As used herein, the term “a staggered or sequential dosing regimen” and related terminology or phraseology such as “a staggered dosing schedule” generally refers to the use of a RSPO-LGR pathway inhibitor in combination with a mitotic inhibitor where the use of or administration of each agent is staggered over time. In some embodiments, the first agent is administered at least about 12, 24, 36, 48, 60, 72, 84, or 96 hours prior to administration of the second agent. In some embodiments, the first agent is administered at least about 1 day, about 2 days, about 3 days, about 4 days, about 5 days, about 6 days, or about 7 days prior to administration of the second agent. In some embodiments, the staggered administration of the two agents includes variations in dosage amounts. As used herein, this definition does not preclude administration of additional therapeutic agents.

In some embodiments, a RSPO-LGR pathway inhibitor (e.g., RSPO-binding agent or LGR-binding agent) in combination with a mitotic inhibitor is used in a method of treating a disease associated with β-catenin signaling, particularly when used in a staggered or sequential dosing regimen. In some embodiments, the disease is dependent upon β-catenin signaling.

In some embodiments, the disease treated with a combination of a RSPO-LGR pathway inhibitor (e.g., RSPO-binding agent or LGR-binding agent) and a mitotic inhibitor, wherein the therapeutic agents are administered using a staggered dosing regimen is cancer. In certain embodiments, the cancer comprises β-catenin signaling dependent tumor cells, or a subset of β-catenin signaling dependent tumor cells. In certain embodiments, the cancer is characterized by tumor cells, or a subset of tumor cells expressing or over-expressing β-catenin. In certain embodiments, the cancer is characterized by tumor cells, or a subset of tumor cells expressing or over-expressing one or more RSPO proteins. In certain embodiments, the cancer is characterized by tumor cells, or a subset of tumor cells expressing or over-expressing one or more LGR proteins.

Described herein is a method of treating cancer comprising administering to a subject a therapeutically effective amount of a RSPO-LGR pathway inhibitor and a therapeutically effective amount of a mitotic inhibitor, wherein the RSPO-LGR pathway inhibitor is administered first and the mitotic inhibitor is administered second. Described herein is a method of treating cancer comprising administering to a subject a therapeutically effective amount of a RSPO-LGR pathway inhibitor and a therapeutically effective amount of a mitotic inhibitor, wherein the RSPO-LGR pathway inhibitor and the mitotic inhibitor are administered using a staggered dosing schedule and the RSPO-LGR pathway inhibitor is administered first. In some embodiments, the mitotic inhibitor is administered about 1 day, about 2 days, about 3 days, about 4 days, about 5 days, about 6 days, or about 7 days after the RSPO-LGR pathway inhibitor is administered. Also described herein is a method of increasing the efficacy of a mitotic inhibitor in treating cancer in a subject comprising administering to the subject a therapeutically effective amount of a mitotic inhibitor about 1 day, about 2 days, about 3 days, about 4 days, about 5 days, or about 6 days after a therapeutically effective amount of a RSPO-LGR pathway inhibitor is administered. Further described herein is a method of increasing the efficacy of a mitotic inhibitor in treating cancer in a subject comprising administering to the subject a therapeutically effective amount of a RSPO-LGR pathway inhibitor, wherein the subject is scheduled to be administered a therapeutically effective amount of a mitotic inhibitor about 1 day, about 2 days, about 3 days, about 4 days, about 5 days, or about 6 days after the RSPO-LGR pathway inhibitor is administered. In some embodiments, the increase in the efficacy of a mitotic inhibitor in treating cancer is relative to the efficacy of the mitotic inhibitor used without the RSPO-LGR pathway inhibitor. In some embodiments, the increase in the efficacy of a mitotic inhibitor in treating cancer is relative to the efficacy observed when the mitotic inhibitor and the RSPO-LGR pathway inhibitor are administered to the patient substantially simultaneously, e.g., on the same day. In some embodiments, a method of treating cancer comprises administering to a subject a therapeutically effective amount of a RSPO-LGR pathway inhibitor and a therapeutically effective amount of a mitotic inhibitor, wherein the RSPO-LGR pathway inhibitor and the mitotic inhibitor are administered using a staggered dosing schedule and the RSPO-LGR pathway inhibitor is administered first; and wherein the RSPO-LGR pathway inhibitor is an antibody that specifically binds at least one human RSPO protein, an antibody that specifically binds at least one human LGR protein, or a soluble receptor comprising the extracellular domain of a human LGR protein or a fragment thereof. In some embodiments, the mitotic inhibitor is administered about 1, 2, 3, 4, 5, 6, or 7 days after the RSPO-LGR pathway inhibitor is administered. In some embodiments, the mitotic inhibitor is administered about 2 days after the RSPO-LGR pathway inhibitor is administered. In some embodiments, the mitotic inhibitor is administered about 3 days after the RSPO-LGR pathway inhibitor is administered.

In some embodiments, a method comprises the use of a RSPO-LGR pathway inhibitor and a mitotic inhibitor for the treatment of cancer, wherein the RSPO-LGR pathway inhibitor and the mitotic inhibitor are used in a staggered dosing schedule and the RSPO-LGR pathway inhibitor is used first; and wherein the RSPO-LGR pathway inhibitor is an antibody that specifically binds at least one human RSPO protein, an antibody that specifically binds at least one human LGR protein, or a soluble receptor comprising the extracellular domain of a human LGR protein or a fragment thereof.

Described herein is a method of increasing the efficacy of a mitotic inhibitor in treating cancer in a subject comprising: (a) administering to the subject a RSPO-LGR pathway inhibitor; and (b) administering to the subject a mitotic inhibitor about 1 day, about 2 days, about 3 days, about 4 days, about 5 days, or about 6 days after the RSPO-LGR pathway inhibitor is administered. In some embodiments, a method of increasing the efficacy of a mitotic inhibitor in treating cancer in a subject comprises administering to the subject a mitotic inhibitor about 1, 2, 3, 4, 5, or 6 days after a RSPO-LGR pathway inhibitor is administered, wherein the RSPO-LGR pathway inhibitor is an antibody that specifically binds at least one human RSPO protein, an antibody that specifically binds at least one human LGR protein, or a soluble receptor comprising the extracellular domain of a human LGR protein or a fragment thereof. In some embodiments, a method of increasing the efficacy of a mitotic inhibitor in treating cancer in a subject comprises: (a) administering to the subject a RSPO-LGR pathway inhibitor, wherein the RSPO-LGR pathway inhibitor is: (i) an antibody that specifically binds at least one human RSPO protein, (ii) an antibody that specifically binds at least one human LGR protein, or (iii) a soluble receptor comprising the extracellular domain of a human LGR protein or a fragment thereof; and (b) administering to the subject a mitotic inhibitor about 1, 2, 3, 4, 5, or 6 days after the RSPO-LGR pathway inhibitor is administered. In some embodiments, the increase in the efficacy of a mitotic inhibitor in treating cancer is relative to the efficacy of the mitotic inhibitor used without the RSPO-LGR pathway inhibitor. In some embodiments, the increase in the efficacy of a mitotic inhibitor in treating cancer is relative to the efficacy observed when the mitotic inhibitor and the RSPO-LGR pathway inhibitor are administered to the patient substantially simultaneously, e.g., on the same day.

In some embodiments, a method of increasing the efficacy of a mitotic inhibitor for the treatment of cancer comprises the use of a mitotic inhibitor about 1, 2, 3, 4, 5, or 6 days after a RSPO-LGR pathway inhibitor is used, wherein the RSPO-LGR pathway inhibitor an antibody that specifically binds at least one human RSPO protein, an antibody that specifically binds at least one human LGR protein, or a soluble receptor comprising the extracellular domain of a human LGR protein or a fragment thereof. In some embodiments, the increase in the efficacy of a mitotic inhibitor in treating cancer is relative to the efficacy of the mitotic inhibitor used without the RSPO-LGR pathway inhibitor. In some embodiments, the increase in the efficacy of a mitotic inhibitor in treating cancer is relative to the efficacy observed when the mitotic inhibitor and the RSPO-LGR pathway inhibitor are administered to the patient substantially simultaneously, e.g., on the same day.

Described herein is a method of improving the efficacy of combination therapy using a RSPO-LGR pathway inhibitor and a mitotic inhibitor, wherein the method comprises administering the mitotic inhibitor after allowing sufficient time for the RSPO-LGR pathway inhibitor to reach its target. In some embodiments, the method of improving the efficacy comprises administering the mitotic inhibitor after allowing sufficient time for the RSPO-LGR pathway inhibitor to accumulate at its target. In some embodiments, the target is a RSPO protein. In some embodiments, the target is an LGR protein. In some embodiments, the target is found associated with a tumor.

In some embodiments of the methods described herein, the mitotic inhibitor is administered about 1 day after the RSPO-LGR pathway inhibitor is administered. In some embodiments, the mitotic inhibitor is administered about 2 days after the RSPO-LGR pathway inhibitor is administered. In some embodiments, the mitotic inhibitor is administered about 3 days after the RSPO-LGR pathway inhibitor is administered.

In some embodiments of the methods described herein, the RSPO-LGR pathway inhibitor and the mitotic inhibitor act synergistically. In some embodiments, the RSPO-LGR pathway inhibitor sensitizes cancer cells to the mitotic inhibitor. In some embodiments, the RSPO-LGR pathway inhibitor sensitizes cancer stem cells to the mitotic inhibitor. In some embodiments, the RSPO-LGR pathway inhibitor suppresses or arrests cell cycle progression during the mitosis (M) phase. In some embodiments, the RSPO-LGR pathway inhibitor suppresses or arrests cell cycle progression at the G2/M checkpoint. In some embodiments, the RSPO-LGR pathway inhibitor suppresses or arrests cell cycle progression at the G2/M checkpoint and increases the efficacy of the mitotic inhibitor. In some embodiments, the RSPO-LGR pathway inhibitor suppresses or arrests cell cycle progression at the M phase and increases the efficacy of the mitotic inhibitor. In some embodiments, the staggered dosing allows for sustained inhibition of β-catenin signaling and increased efficacy of the mitotic inhibitor.

In some embodiments of the methods described herein, the staggered dosing schedule of a RSPO-LGR pathway inhibitor in combination with a mitotic inhibitor increases apoptosis of tumor cells. In some embodiments, the staggered dosing schedule of a RSPO-LGR pathway inhibitor in combination with a mitotic inhibitor allows for accumulation of the RSPO-LGR pathway inhibitor at the tumor site(s). In some embodiments, the staggered dosing schedule of a RSPO-LGR pathway inhibitor in combination with a mitotic inhibitor allows for synchronization of anti-tumor activity of the RSPO-LGR pathway inhibitor and the mitotic inhibitor.

In some embodiments of the methods described here, the RSPO-LGR pathway inhibitor is administered once every week. In some embodiments, the RSPO-LGR pathway inhibitor is administered once every 2 weeks. In some embodiments, the RSPO-LGR pathway inhibitor is administered once every 3 weeks. In some embodiments, the RSPO-LGR pathway inhibitor is administered once every 4 weeks. In some embodiments, the mitotic inhibitor is administered about once a week, about once every 2 weeks, about once every 3 weeks, about once every 4 weeks, or about once every 3 weeks out of a 4 week cycle. In some embodiments, the RSPO-LGR pathway inhibitor is administered about once every 2 weeks and the mitotic inhibitor is administered once a week or once a week for 3 weeks of a 4 week cycle. In some embodiments, the RSPO-LGR pathway inhibitor is administered about once every 3 weeks and the mitotic inhibitor is administered once a week or once a week for 3 weeks of a 4 week cycle. In some embodiments, the RSPO-LGR pathway inhibitor is administered once every 4 weeks. In some embodiments, the mitotic inhibitor is administered about once a week, about once every 2 weeks, about once every 3 weeks, or about once every 4 weeks. In some embodiments, the RSPO-LGR pathway inhibitor is administered once every 4 weeks and the mitotic inhibitor is administered once a week or once a week for 3 weeks of a 4 week cycle.

In some embodiments, a treatment or dosing regimen can be limited to a specific number of administrations or “cycles”. A “cycle” can be a dosing schedule that is well-known or commonly used by those of skill in the art for a standard-of-care therapeutic agent. For example, a cycle of paclitaxel can be administration once a week for 3 weeks of a 4 week (28 day) cycle (there is one week of no administration every 4 weeks). In some embodiments, the RSPO-LGR pathway inhibitor is administered for 2, 3, 4, 5, 6, 7, 8, or more cycles. In some embodiments, the mitotic inhibitor is administered for 2, 3, 4, 5, 6, 7, 8, or more cycles. In some embodiments, one agent is withheld for 1 or more cycles while administration of the second agent is continued.

In some embodiments of the methods described herein, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, bladder cancer, glioblastoma, and head and neck cancer. In some embodiments, the cancer contains a RSPO gene fusion. In some embodiments, the cancer contains a RSPO2 gene fusion. In some embodiments, the cancer contains a RSPO3 gene fusion. In certain embodiments, the cancer is breast cancer. In some embodiments, the cancer is ovarian cancer. In certain embodiments, the cancer is pancreatic cancer. In certain embodiments, the cancer is lung cancer. As used herein, “lung cancer” includes but is not limited to, small cell lung carcinoma and non-small cell lung carcinoma (NSCLC). In certain embodiments, the cancer is colorectal cancer. In some embodiments, the cancer is colorectal cancer that comprises an inactivating mutation in the APC gene. In some embodiments, the cancer is colorectal cancer that does not comprise an inactivating mutation in the APC gene. In some embodiments, the cancer comprises an activating mutation in the β-catenin gene. In some embodiments, the cancer does not comprise an activating mutation in the β-catenin gene. In some embodiments, the tumor comprises an activating mutation in the β-catenin gene. In some embodiments, the cancer is colorectal cancer that contains a RSPO gene fusion. In some embodiments, the cancer is colorectal cancer that contains a RSPO2 gene fusion. In some embodiments, the cancer is colorectal cancer that contains a RSPO3 gene fusion. In some embodiments, the cancer has elevated expression level of a RSPO polypeptide. In some embodiments, the cancer has elevated expression level of RSPO1, RSPO2, RSPO3, and/or RSPO4. In some embodiments, the cancer is colorectal cancer with an elevated expression level of RSPO3. In some embodiments, the cancer is colorectal cancer with an elevated expression level of RSPO2. In some embodiments, the cancer does not have elevated expression level of a RSPO polypeptide. In some embodiments, the cancer does not have elevated expression level of RSPO1, RSPO2, RSPO3, and/or RSPO4. In some embodiments, the cancer is colorectal cancer that does not have elevated expression level of RSPO3. In some embodiments, the cancer is colorectal cancer that does not have elevated expression level of RSPO2. In some embodiments, the cancer has substantially the same expression level of a RSPO polypeptide as normal tissue of the same tissue type. In some embodiments, the cancer has substantially the same expression level of RSPO1, RSPO2, RSPO3, and/or RSPO4 as normal tissue of the same tissue type. In some embodiments, the cancer is colorectal cancer that has substantially the same expression level of RSPO3 as normal tissue of the same tissue type. In some embodiments, the cancer is colorectal cancer that has substantially the same expression level of RSPO2 as normal tissue of the same tissue type.

In some embodiments, the cancer is a colorectal cancer that comprises a mutation in a gene encoding a component of the Wnt signaling pathway. See, for example, U.S. Patent Publication No. 20130209473, which is hereby incorporated by reference herein in its entirety for all purposes. In some embodiments, the cancer is a colorectal cancer that comprises a mutation in a Wnt (e.g., WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11, WNT16), Frizzled (e.g., FZD1-FZD10), RSPO (e.g., RSPO1, RSPO2, RSPO3, RSPO4), LGR (e.g., LGR4, LGR5, LGR6), WTX, WISP (e.g., WISP1, WISP2, WISP3), β-TrCp, STRA6, LRP (e.g., LRP5, LRP6), Axin (e.g., AXIN1, AXIN2), Dishevelled, sFRP, WIF-1, Dkk, Krn, GSK3β, CKIα, PP2A, pygopus, bc19/legless, TCF/LEF, Groucho, CTNNB1, CBP/p300, Brg-1 genes, TCFL2, PPN, CDH17, EZH2, HMGA1, HMGA2, YY1, and/or TC1 gene. In certain embodiments, the Wnt signaling pathway is activated in the colorectal cancer which comprises the mutation.

In some embodiments, the colorectal cancer treated with the RSPO-LGR pathway inhibitor (e.g., anti-RSPO3 or anti-LGR5 antibody) and mitotic inhibitor is second-line or third-line colorectal cancer. In certain embodiments, the colorectal cancer is resistant to treatment with a chemotherapy regimen. In certain embodiments, the colorectal cancer is resistant to a chemotherapy treatment comprising one or more of 5-fluorouracil (5-FU), irinotecan, and/or oxaliplatin. In some embodiments, the colorectal cancer is resistant to irinotecan/5-FU/leucovorin (FOLFIRI) and/or oxaliplatin/5-FU/leucovorin (FOLFOX). In certain alternative embodiments, the colorectal cancer is resistant to a treatment with bevacizumab. In certain embodiments, the patient treated with the RSPO-LGR pathway inhibitor and mitotic inhibitor has failed one prior treatment regimen. In another embodiment the patient treated with the RSPO-LGR pathway inhibitor and mitotic inhibitor has failed two prior treatment regimens. In certain embodiments the prior treatment regimen (or regimens) comprises treatment with one or more of 5-fluorouracil (5-FU), irinotecan, oxaliplatin and/or bevacizumab.

In some embodiments, a method of treating cancer comprises administering to a subject a therapeutically effective amount of anti-RSPO3 antibody OMP-131R010 and a therapeutically effective amount of a taxane selected from the group consisting of paclitaxel, nab-paclitaxel, and docetaxel, wherein the taxane is administered about 1, 2, 3, 4, 5, 6 or 7 days after OMP-131R010 is administered. In some embodiments, OMP-131R010 is administered about once a week. In some embodiments, OMP-131R010 is administered about once every 2 weeks. In some embodiments, OMP-131R010 is administered about once every 3 weeks. In some embodiments, OMP-131R010 is administered about once every 4 weeks. In some embodiments, taxane is administered once a week. In some embodiments, taxane is administered once every 2 weeks. In some embodiments, taxane is administered once every three weeks. In some embodiments, taxane is administered once a week for 3 weeks of a 4 week cycle. In some embodiments, a method of treating cancer comprises administering to a subject a therapeutically effective amount of OMP-131R010 and a therapeutically effective amount of docetaxel, wherein the docetaxel is administered about 2 or 3 days after OMP-131R010 is administered. In some embodiments, a method of treating cancer comprises administering to a subject a therapeutically effective amount of OMP-131R010, a therapeutically effective amount of nab-paclitaxel, and a therapeutically effective amount of gemcitabine, wherein the nab-paclitaxel is administered about 2 or 3 days after OMP-131R010 is administered. In some embodiments, a method of treating cancer comprises administering to a subject a therapeutically effective amount of OMP-131R010, a therapeutically effective amount of nab-paclitaxel, and a therapeutically effective amount of gemcitabine, wherein the nab-paclitaxel and the gemcitabine are administered about 2 or 3 days after OMP-131R010 is administered. In some embodiments, a method of treating cancer comprises administering to a subject a therapeutically effective amount of OMP-131R010 and a therapeutically effective amount of paclitaxel, wherein the paclitaxel is administered about 2 or 3 days after OMP-131R010 is administered.

Described herein is a method of inhibiting tumor growth or reducing tumor size comprising contacting tumor cells with an effective amount of a RSPO-LGR pathway inhibitor and an effective amount of a mitotic inhibitor, wherein the RSPO-LGR pathway inhibitor is administered to the cells first and the mitotic inhibitor is administered to the cells second. Described herein is a method of inhibiting tumor growth or reducing tumor size comprising contacting tumor cells with an effective amount of a RSPO-LGR pathway inhibitor and an effective amount of a mitotic inhibitor, wherein the RSPO-LGR pathway inhibitor and the mitotic inhibitor are administered to the cells using a staggered dosing schedule and the RSPO-LGR pathway inhibitor is administered to the cells first. In some embodiments, the mitotic inhibitor is administered about 12, 24, 36, 48, 60, 72, 84, or 96 hours after the RSPO-LGR pathway inhibitor is administered. In some embodiments, the mitotic inhibitor is administered about 1 day, about 2 days, about 3 days, about 4 days, about 5 days, about 6 days, or about 7 days after the RSPO-LGR pathway inhibitor is administered. Described herein is a method of increasing the efficacy of a mitotic inhibitor in inhibiting tumor growth or reducing tumor size comprising: (a) contacting tumor cells with a RSPO-LGR pathway inhibitor; and (b) contacting the tumor cells with a mitotic inhibitor about 1 day, about 2 days, about 3 days, about 4 days, about 5 days, about 6 days, or about 7 days after the RSPO-LGR pathway inhibitor is administered. In some embodiments, the increase in the efficacy of a mitotic inhibitor in treating cancer is relative to the efficacy of the mitotic inhibitor used without the RSPO-LGR pathway inhibitor. In some embodiments, the increase in the efficacy of a mitotic inhibitor in treating cancer is relative to the efficacy observed when the mitotic inhibitor and the RSPO-LGR pathway inhibitor are administered to the patient substantially simultaneously, e.g., on the same day.

In certain embodiments of the methods described herein, the method of inhibiting tumor growth or reducing tumor size comprises contacting the tumor or tumor cell with a RSPO-LGR pathway inhibitor and a mitotic pathway inhibitor in vitro. For example, in some embodiments, an immortalized cell line or a cancer cell line is cultured in medium to which is added the RSPO-LGR pathway inhibitor followed by addition of the mitotic inhibitor to inhibit tumor cell growth. In some embodiments, tumor cells are isolated from a patient sample such as, for example, a tissue biopsy, pleural effusion, or blood sample and cultured in medium to which is added the RSPO-LGR pathway inhibitor and a mitotic inhibitor to inhibit tumor cell growth.

In some embodiments, the method of inhibiting tumor growth or reducing tumor size comprises contacting the tumor or tumor cells with a RSPO-LGR pathway inhibitor and a mitotic inhibitor in vivo. In certain embodiments, contacting a tumor or tumor cell with a RSPO-LGR pathway inhibitor and a mitotic inhibitor is undertaken in an animal model. For example, a RSPO-LGR pathway inhibitor and a mitotic inhibitor can be administered in a staggered dosing manner to immunocompromised mice (e.g., NOD/SCID mice) which bear xenograft tumors to inhibit growth of the tumors. In certain embodiments, cancer stem cells are isolated from a patient sample such as, for example, a tissue biopsy, pleural effusion, or blood sample and injected into immunocompromised mice that are then administered in a staggered dosing manner a RSPO-LGR pathway inhibitor followed by administration of a mitotic inhibitor to inhibit tumor cell growth. In some embodiments, a RSPO-LGR pathway inhibitor and a mitotic inhibitor are administered in a staggered dosing manner at the same time or shortly after introduction of cells into the animal to prevent tumor growth (preventative model). In some embodiments, a RSPO-LGR pathway inhibitor and a mitotic inhibitor are administered in a staggered dosing manner after the cells have grown to a tumor of a specific size to inhibit and/or reduce tumor growth (therapeutic model).

Described herein is a method of inhibiting tumor growth or reducing tumor size in a subject, the method comprising administering to the subject a therapeutically effective amount of a RSPO-LGR pathway inhibitor and a therapeutically effective amount of a mitotic inhibitor in a staggered dosing manner, wherein the RSPO-LGR pathway inhibitor is administered prior to administration of the mitotic inhibitor. In certain embodiments, the subject is a human. In certain embodiments, the subject has a tumor or has had a tumor removed. In some embodiments, the subject has a tumor that has metastasized. In some embodiments, the subject has had prior therapeutic treatment.

Described herein is a method of inhibiting invasiveness of a tumor in a subject, the method comprising administering to the subject a therapeutically effective amount of a RSPO-LGR pathway inhibitor and a therapeutically effective amount of a mitotic inhibitor in a staggered dosing manner, wherein the RSPO-LGR pathway inhibitor is administered prior to administration of the mitotic inhibitor. In some embodiments, the inhibition of invasiveness comprises increasing E-cadherin expression of the tumor cells. In certain embodiments, the subject is a human. In certain embodiments, the subject has a tumor or has had a tumor removed.

Described herein is a method of reducing or preventing metastasis in a subject, the method comprising administering to the subject a therapeutically effective amount of a RSPO-LGR pathway inhibitor and a therapeutically effective amount of a mitotic inhibitor in a staggered dosing manner, wherein the RSPO-LGR pathway inhibitor is administered prior to administration of the mitotic inhibitor. In some embodiments, the reduction or prevention of metastasis comprises inhibiting invasiveness of a tumor. In some embodiments, the reduction or prevention of metastasis comprises inhibiting invasiveness of a tumor by increasing E-cadherin expression of the tumor cells. In certain embodiments, the subject is a human. In certain embodiments, the subject has a tumor or has had a tumor removed.

Described herein is a method of inhibiting β-catenin signaling in a cell, the method comprising contacting the cell with an effective amount of a RSPO-LGR pathway inhibitor and an effective amount of a mitotic inhibitor in a staggered dosing manner, wherein the RSPO-LGR pathway inhibitor is administered prior to administration of the mitotic inhibitor. In certain embodiments, the cell is a tumor cell. In certain embodiments, the method is an in vivo method wherein the step of contacting the cell with the inhibitor(s) comprises administering a therapeutically effective amount of the inhibitor(s) to a subject. In some embodiments, the method is an in vitro or ex vivo method.

In addition, described herein is a method of reducing the tumorigenicity of a tumor in a subject, the method comprising administering to the subject a therapeutically effective amount of a RSPO-LGR pathway inhibitor and a therapeutically effective amount of a mitotic inhibitor in a staggered dosing manner, wherein the RSPO-LGR pathway inhibitor is administered prior to administration of the mitotic inhibitor. In certain embodiments, the tumor comprises cancer stem cells. In some embodiments, the tumorigenicity of a tumor is reduced by reducing the frequency of cancer stem cells in the tumor. In certain embodiments, the frequency of cancer stem cells in the tumor is reduced by administration of the RSPO-LGR pathway inhibitor. In some embodiments, the tumorigenicity of the tumor is reduced by inducing differentiation of the tumor cells. In some embodiments, the tumorigenicity of the tumor is reduced by inducing apoptosis of the tumor cells. In some embodiments, the tumorigenicity of the tumor is reduced by increasing apoptosis of the tumor cells.

Described herein is a method of reducing cancer stem cell frequency in a tumor comprising cancer stem cells, the method comprising administering to a subject a therapeutically effective amount of a RSPO-LGR pathway inhibitor and a therapeutically effective amount of a mitotic inhibitor in a staggered dosing manner, wherein the RSPO-LGR pathway inhibitor is administered prior to administration of the mitotic inhibitor. In certain embodiments, the RSPO-LGR pathway inhibitor in combination with a mitotic inhibitor is capable of reducing the tumorigenicity of a tumor comprising cancer stem cells in an animal model, such as a mouse xenograft model. In certain embodiments, the number or frequency of cancer stem cells in a treated tumor is reduced by at least about two-fold, about three-fold, about five-fold, about ten-fold, about 50-fold, about 100-fold, or about 1000-fold as compared to the number or frequency of cancer stem cells in an untreated tumor. In certain embodiments, the reduction in the number or frequency of cancer stem cells is determined by limiting dilution assay using an animal model.

In certain embodiments, the tumor is a tumor in which β-catenin signaling is active. In certain embodiments, the tumor is a β-catenin signaling dependent tumor. In some embodiments, the tumor is a tumor in which β-catenin signaling is aberrant. In certain embodiments, the tumor comprises an inactivating mutation (e.g., a truncating mutation) in the APC tumor suppressor gene. In certain embodiments, the tumor does not comprise an inactivating mutation in the APC tumor suppressor gene. In some embodiments, the tumor comprises a wild-type APC gene. In some embodiments, the tumor comprises an activating mutation in the β-catenin gene. In some embodiments, the tumor does not comprise an activating mutation in the β-catenin gene. In certain embodiments, a cancer for which a subject is being treated involves such a tumor.

In some embodiments, the tumor comprises a RSPO gene fusion. In some embodiments, the tumor comprises a RSPO2 gene fusion. In some embodiments, the tumor comprises a RSPO3 gene fusion. In certain embodiments, a cancer for which a subject is being treated involves such a tumor.

In certain embodiments of the methods described herein, the tumor expresses one or more human RSPO proteins to which a RSPO-binding agent binds. In certain embodiments, the tumor over-expresses one or more human RSPO protein(s). In certain embodiments, the tumor over-expresses one or more human RSPO protein(s) as compared to the RSPO protein expression in normal tissue of the same tissue type. In certain embodiments, the tumor over-expresses one or more human RSPO protein(s) as compared to the RSPO protein expression in at least one other tumor. In some embodiments, the tumor over-expresses RSPO1, RSPO2, RSPO3, and/or RSPO4. In some embodiments, the tumor over-expresses RSPO1 or RSPO3. In certain embodiments, the tumor does not over-express one or more human RSPO protein(s). In certain embodiments, the tumor does not over-express one or more human RSPO protein(s) as compared to the RSPO protein expression in normal tissue of the same tissue type. In some embodiments, the tumor does not over-express RSPO1, RSPO2, RSPO3, and/or RSPO4. In some embodiments, the tumor expresses RSPO1, RSPO2, RSPO3, and/or RSPO4 substantially at the same level as normal tissue of the same tissue type. In some embodiments, the tumor expresses low RSPO1, RSPO2, RSPO3, and/or RSPO4 levels compared to a pre-determined expression level. In some embodiments, the tumor expresses high RSPO1, RSPO2, RSPO3, and/or RSPO4 levels compared to a pre-determined expression level. In some embodiments, the pre-determined expression level of RSPO1, RSPO2, RSPO3, or RSPO4 is the expression level of RSPO1, RSPO2, RSPO3, or RSPO4 in a tumor or a group of tumors of the same tissue type. In some embodiments, the pre-determined RSPO1, RSPO2, RSPO3, or RSPO4 expression level is the expression level of RSPO1, RSPO2, RSPO3, or RSPO4 in a tumor or group of tumors of a different tissue type. In certain embodiments, a cancer for which a subject is being treated involves such a tumor.

In certain embodiments, the tumor expresses one or more human LGR proteins to which a LGR-binding agent binds. In certain embodiments, the tumor over-expresses one or more human LGR proteins. In certain embodiments, the tumor over-expresses human LGR5.

In some embodiments of the methods described herein, the tumor is a tumor selected from the group consisting of colorectal tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In some embodiments, the tumor contains a RSPO gene fusion. In some embodiments, the tumor contains a RSPO2 gene fusion. In some embodiments, the tumor contains a RSPO3 gene fusion. In certain embodiments, the tumor is a breast tumor. In some embodiments, the tumor is an ovarian tumor. In certain embodiments, the tumor is a lung tumor. In certain embodiments, the tumor is a pancreatic tumor. In certain embodiments, the tumor is a colorectal tumor. In some embodiments, the tumor is a colorectal tumor in which Wnt signaling is activated (e.g., by a mutation in a component of the Wnt signaling pathway). In some embodiments, the tumor is a colorectal tumor that comprises an inactivating mutation in the APC gene. In some embodiments, the tumor is a colorectal tumor that does not comprise an inactivating mutation in the APC gene. In some embodiments, the tumor comprises an activating mutation in the β-catenin gene. In some embodiments, the tumor is a colorectal tumor that contains a RSPO gene fusion. In some embodiments, the tumor is a colorectal tumor that contains a RSPO2 gene fusion. In some embodiments, the tumor is a colorectal tumor that contains a RSPO3 gene fusion. In some embodiments, the tumor has elevated expression level of a RSPO polypeptide. In some embodiments, the tumor has an elevated expression level of RSPO1, RSPO2, RSPO3, and/or RSPO4. In some embodiments, the tumor is a colorectal tumor with an elevated expression level of RSPO3. In some embodiments, the tumor is a colorectal tumor with an elevated expression level of RSPO2. In some embodiments, the tumor does not have an elevated expression level of a RSPO polypeptide. In some embodiments, the tumor does not have an elevated expression level of RSPO1, RSPO2, RSPO3, and/or RSPO4. In some embodiments, the tumor is a colorectal tumor that does not have an elevated expression level of RSPO3. In some embodiments, the tumor is a colorectal tumor that does not have an elevated expression level of RSPO2. In some embodiments, the tumor has substantially the same expression level of a RSPO polypeptide as normal tissue of the same tissue type. In some embodiments, the tumor has substantially the same expression level of RSPO1, RSPO2, RSPO3, and/or RSPO4 as normal tissue of the same tissue type. In some embodiments, the tumor is a colorectal tumor that has substantially the same expression level of RSPO3 as normal tissue of the same tissue type. In some embodiments, the tumor is a colorectal tumor that has substantially the same expression level of RSPO2 as normal tissue of the same tissue type. In some embodiments, the tumor is a colorectal tumor that expresses low RSPO1, RSPO2, RSPO3, and/or RSPO4 levels compared to a pre-determined expression level. In some embodiments, the tumor is a colorectal tumor that expresses high RSPO1, RSPO2, RSPO3, and/or RSPO4 levels compared to a pre-determined expression level. In some embodiments, the pre-determined RSPO1, RSPO2, RSPO3, or RSPO4 expression level is the expression level of RSPO1, RSPO2, RSPO3, or RSPO4 in normal tissue of the same tissue type. In some embodiments, the pre-determined RSPO1, RSPO2, RSPO3, or RSPO4 expression level is the expression level of RSPO1, RSPO2, RSPO3, or RSPO4 in a tumor or a group of tumors of the same tissue type. In some embodiments, the pre-determined RSPO1, RSPO2, RSPO3, or RSPO4 expression level is the expression level of RSPO1, RSPO2, RSPO3, or RSPO4 in a tumor or group of tumors of a different tissue type.

The phrases “a tumor has elevated expression levels of,” “a tumor has substantially the same expression level as normal tissue of the same tissue type,” “a tumor has low RSPO3 expression,” or “a tumor has high RSPO3 expression” may refer to expression levels of a protein or expression levels of a nucleic acid. In general, the phrase “a tumor has elevated expression levels of,” “a tumor has high expression levels of,” “a tumor has low expression levels of,” or “a tumor has substantially the same expression levels of” a protein or a gene (or similar phrases) refers to expression levels of a protein or a gene in a tumor as compared to expression levels of the same protein or the same gene in a reference sample or to a pre-determined expression level. In some embodiments, the reference sample is normal tissue of the same tissue type. In some embodiments, the reference sample is normal tissue of a group of tissue types. In some embodiments, the reference sample is a tumor or a group of tumors of the same tissue type. In some embodiments, the reference sample is a tumor or group of tumors of a different tissue type. Thus in some embodiments, the expression levels of a protein or a gene in a tumor are “elevated,” “high,” “low,” or “substantially the same” as compared to the average expression level of the protein or the gene within a group of tissue types. In some embodiments, the expression levels of a protein or a gene in a tumor are “elevated,” “high,” “low,” or “substantially the same” as compared to the expression level of the protein or the gene in other tumors of the same tissue type or a different tissue type. In some embodiments, the tumor expresses “elevated,” “high,” “low,” or “substantially the same” levels of RSPO1, RSPO2, RSPO3, and/or RSPO4 as compared to the RSPO levels expressed in normal tissue of the same tissue type. In some embodiments, the tumor expresses “elevated,” “high,” or “substantially the same” levels of RSPO1, RSPO2, RSPO3, and/or RSPO4 as compared to a pre-determined level.

In certain embodiments, a method described herein further comprises a step of determining the expression level of at least one RSPO (i.e., protein or nucleic acid) in the tumor or cancer. In some embodiments, the step of determining the expression level of a RSPO in the tumor or cancer comprises determining the expression level of one or more of RSPO1, RSPO2, RSPO3, and RSPO4. In some embodiments, the expression level of one or more of RSPO1, RSPO2, RSPO3, and RSPO4 in a tumor or cancer is compared to the expression level of one or more of RSPO1, RSPO2, RSPO3, and RSPO4 in a reference sample. In some embodiments, the expression level of one or more of RSPO1, RSPO2, RSPO3, and RSPO4 in a tumor or cancer is compared to the expression level of RSPO1, RSPO2, RSPO3, and RSPO4, respectively, in normal tissue of the same tissue type. In some embodiments, the level of expression of one or more of RSPO1, RSPO2, RSPO3, and RSPO4 in a tumor or cancer is compared to a pre-determined level of expression of RSPO1, RSPO2, RSPO3, and RSPO4, respectively. In some embodiments, the level of expression of one or more of RSPO1, RSPO2, RSPO3, and RSPO4 in a tumor or cancer is compared to a pre-determined level of expression of RSPO1, RSPO2, RSPO3, and RSPO4, respectively, in normal tissue of the same tissue type. In some embodiments, the tumor or cancer has elevated expression of one or more of RSPO1, RSPO2, RSPO3, and RSPO4. In some embodiments, the tumor or cancer does not have elevated expression of one or more of RSPO1, RSPO2, RSPO3, and RSPO4. In some embodiments, the tumor or cancer expresses one or more of RSPO1, RSPO2, RSPO3, and RSPO4 substantially at the same level as the reference sample. In general, the expression level of a RSPO (i.e., protein or nucleic acid) is compared to the expression level of the RSPO (i.e., protein or nucleic acid) in normal tissue of the same tissue type. However, in some embodiments, the expression level of a RSPO (i.e., protein or nucleic acid) is compared to the average expression level of the RSPO (i.e., protein or nucleic acid) within a group of tissue types. In some embodiments, the expression levels of a RSPO (i.e., protein or nucleic acid) in a tumor is compared to the expression level of the RSPO (i.e., protein or nucleic acid) in other tumors of the same tissue type or a different tissue type. In some embodiments, determining the level of RSPO expression is done prior to treatment with the RSPO-LGR pathway inhibitor.

In certain embodiments, a method described herein further comprises a step of determining if the tumor or cancer has an inactivating mutation in the APC gene. In some embodiments, a method described herein further comprises a step of determining if the tumor or cancer has an activating mutation in the β-catenin gene. In some embodiments, a method described herein further comprises a step of determining if the tumor or cancer has a mutation in a gene encoding a component of the Wnt signaling pathway. See, e.g., Seahgiri et al., Nature, 488: 660-664 (2012) and U.S. Patent Publication No. 20130209473, each of which is hereby incorporated by reference herein in its entirety for all purposes. In some embodiments, a method described herein further comprises a step of determining if the tumor or cancer has a mutation in a Wnt (e.g., WNT1, WNT2, WNT2B, WNT3, WNT3A, WNT4, WNT5A, WNT5B, WNT6, WNT7A, WNT7B, WNT8A, WNT8B, WNT9A, WNT9B, WNT10A, WNT10B, WNT11, WNT16), Frizzled (e.g., Frz 1-10), RSPO (e.g., RSPO1, RSPO2, RSPO3, RSPO4), LGR (e.g., LGR4, LGR5, LGR6), WTX, WISP (e.g., WISP1, WISP2, WISP3), β-TrCp, STRA6, LRP (e.g., LRP5, LRP6), Axin (e.g., AXIN1, AXIN2), Dishevelled, sFRP, WIF-1, Dkk, Krn, GSK3β, CK1α, PP2A, pygopus, bc19/legless, TCF/LEF, Groucho, CTNNB1, CBP/p300, Brg-1 genes, TCFL2, PPN, CDH17, EZH2, HMGA1, HMGA2, YY1, and/or TC1 gene.

In certain embodiments, a method described herein further comprises a step of determining if the tumor or cancer has a RSPO gene fusion.

In certain embodiments, a method described herein further comprises a step of determining the level of RSPO1, RSPO2, RSPO3, and/or RSPO4 expression in the tumor or cancer. In some embodiments, determining the level of RSPO expression is done prior to treatment with the RSPO-LGR pathway inhibitor. In some embodiments, the subject is administered the RSPO-LGR pathway inhibitor if the tumor or cancer has an inactivating mutation in the APC gene.

Methods for determining the level of RSPO expression in a cell, tumor, or cancer are known by those of skill in the art. For nucleic acid expression these methods include, but are not limited to, PCR-based assays, microarray analyses and nucleotide sequencing (e.g., NextGen sequencing). For protein expression these methods include, but are not limited to, Western blot analyses, protein arrays, ELISAs, immunohistochemistry (IHC) assays, and FACS.

Methods for determining whether a tumor has a RSPO gene fusion or a mutation in a gene encoding a RSPO-LGR pathway component are known by those of skill in the art. Methods may include but are not limited to, PCR-based assays, microarray analyses, and nucleotide sequencing (e.g., NextGen sequencing, whole-genome sequencing (WGS)).

Methods for determining the level of RSPO expression, presence of a RSPO gene fusion, or the presence of a mutation in a gene encoding a RSPO-LGR pathway component can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA.

In some embodiments of any of the methods described herein, the RSPO-LGR pathway inhibitor is a RSPO-binding agent. In some embodiments, the RSPO-LGR pathway inhibitor is a LGR-binding agent. In some embodiments, the RSPO-LGR pathway inhibitor is an antibody. In some embodiments, the RSPO-LGR pathway inhibitor is an anti-RSPO antibody. In some embodiments, the RSPO-LGR pathway inhibitor is an anti-RSPO3 antibody. In some embodiments, the RSPO-LGR pathway inhibitor is an anti-LGR antibody. In some embodiments, the RSPO-LGR pathway inhibitor is an anti-LGR5 antibody. In some embodiments, the RSPO-LGR pathway inhibitor is the antibody OMP-131R010. In some embodiments, the RSPO-LGR pathway inhibitor is a soluble receptor. In some embodiments, the RSPO-LGR pathway inhibitor is a LGR soluble receptor. In some embodiments, the RSPO-LGR pathway inhibitor is a LGR-Fc soluble receptor. In some embodiments, the RSPO-LGR pathway inhibitor is a LGR5-Fc soluble receptor. In certain embodiments, the LGR5-Fc soluble receptor comprises the amino acid sequence of SEQ ID NO:63.

In some embodiments of any of the methods described herein, the RSPO-LGR pathway inhibitor is an antibody that specifically binds at least one RSPO protein or fragment thereof. In some embodiments, the antibody specifically binds at least one human RSPO protein selected from the group consisting of: RSPO1, RSPO2, RSPO3, and RSPO4. In some embodiments, the antibody specifically binds human RSPO3. In some embodiments, the RSPO-LGR pathway inhibitor is an antibody that specifically binds RSPO3 and comprises: (a) a heavy chain CDR1 comprising DYSIH (SEQ ID NO:29), a heavy chain CDR2 comprising YIYPSNGDSGYNQKFK (SEQ ID NO:30), and a heavy chain CDR3 comprising ATYFANNFDY (SEQ ID NO:32) or TYFANNFD (SEQ ID NO:31); and (b) a light chain CDR1 comprising KASQSVDYDGDSYMN (SEQ ID NO:33), a light chain CDR2 comprising AASNLES (SEQ ID NO:34), and a light chain CDR3 comprising QQSNEDPLT (SEQ ID NO:36).

In certain embodiments of any of the methods described herein, the RSPO-LGR pathway inhibitor is an antibody comprising (a) a heavy chain CDR1 comprising DYSIH (SEQ ID NO:29), a heavy chain CDR2 comprising YIYPSNGDSGYNQKFK (SEQ ID NO:30), and a heavy chain CDR3 comprising ATYFANNFDY (SEQ ID NO:32) or TYFANNFD (SEQ ID NO:31); and (b) a light chain CDR1 comprising KASQSVDYDGDSYMN (SEQ ID NO:33), a light chain CDR2 comprising AASNLES (SEQ ID NO:34), and a light chain CDR3 comprising QQSNEDPLT (SEQ ID NO:36) and is administered in combination with a mitotic inhibitor in a staggered dosing manner.

In certain embodiments of any of the methods described herein, the RSPO-LGR pathway inhibitor is an antibody comprising (a) a heavy chain CDR1 comprising DYSIH (SEQ ID NO:29), a heavy chain CDR2 comprising YIYPSNGDSGYNQKFK (SEQ ID NO:30), and a heavy chain CDR3 comprising ATYFANNFDY (SEQ ID NO:32) or TYFANNFD (SEQ ID NO:31); and (b) a light chain CDR1 comprising KASQSVDYDGDSYMN (SEQ ID NO:33), a light chain CDR2 comprising AASNLES (SEQ ID NO:34), and a light chain CDR3 comprising QQSNEDPLT (SEQ ID NO:36) and is administered in combination with a taxane in a staggered dosing manner.

In certain embodiments of any of the methods described herein, the RSPO-LGR pathway inhibitor is an antibody comprising (a) a heavy chain CDR1 comprising DYSIH (SEQ ID NO:29), a heavy chain CDR2 comprising YIYPSNGDSGYNQKFK (SEQ ID NO:30), and a heavy chain CDR3 comprising ATYFANNFDY (SEQ ID NO:32) or TYFANNFD (SEQ ID NO:31); and (b) a light chain CDR1 comprising KASQSVDYDGDSYMN (SEQ ID NO:33), a light chain CDR2 comprising AASNLES (SEQ ID NO:34), and a light chain CDR3 comprising QQSNEDPLT (SEQ ID NO:36) and is administered in combination with paclitaxel, nab-paclitaxel, or docetaxel in a staggered dosing manner.

In certain embodiments of any of the methods described herein, the RSPO-LGR pathway inhibitor is an antibody comprising a heavy chain variable region comprising SEQ ID NO:38 and a light chain variable region comprising SEQ ID NO:39, administered in combination with a mitotic inhibitor in a staggered dosing manner.

In certain embodiments of any of the methods described herein, the RSPO-LGR pathway inhibitor is an antibody comprising a heavy chain variable region comprising SEQ ID NO:38 and a light chain variable region comprising SEQ ID NO:39, administered in combination with a taxane in a staggered dosing manner.

In certain embodiments of any of the methods described herein, the RSPO-LGR pathway inhibitor is an antibody comprising a heavy chain variable region comprising SEQ ID NO:38 and a light chain variable region comprising SEQ ID NO:39, administered in combination with paclitaxel, nab-paclitaxel, or docetaxel in a staggered dosing manner.

In some embodiments, the antibody is a monoclonal antibody, a recombinant antibody, a chimeric antibody, a humanized antibody, a human antibody, or an antibody fragment comprising an antigen-binding site. In some embodiments, the antibody is a monospecific antibody or a bispecific antibody. In some embodiments, the antibody is an IgG1 antibody, an IgG2 antibody, or an IgG4 antibody. In some embodiments, the RSPO-LGR pathway inhibitor is antibody OMP-131R010.

In certain embodiments of any of the methods described herein, the RSPO-LGR pathway inhibitor is a soluble receptor. In some embodiments, the soluble receptor comprises an extracellular domain of a LGR protein or a fragment thereof. In some embodiments, the LGR protein is LGR5. In certain embodiments, the extracellular domain comprises amino acids 22-564 of human LGR5 (SEQ ID NO:56).

In certain embodiments of any of the methods described herein, the RSPO-LGR pathway inhibitor is a LGR-Fc soluble receptor comprising amino acids 22-564 of human LGR5, administered in combination with a mitotic inhibitor in a staggered dosing manner. In some embodiments, the mitotic inhibitor is a taxane. In some embodiments, the taxane is paclitaxel, nab-paclitaxel, or docetaxel.

Described herein are compositions comprising a RSPO-LGR pathway inhibitor and/or a mitotic inhibitor. In some embodiments, the composition comprises a RSPO-binding agent or polypeptide described herein. In some embodiments, the composition comprises a LGR-binding agent or polypeptide described herein. In some embodiments, the composition comprises a mitotic inhibitor described herein. In some embodiments, the composition is a pharmaceutical composition comprising a RSPO-LGR pathway inhibitor and a pharmaceutically acceptable vehicle. In some embodiments, the composition is a pharmaceutical composition comprising a mitotic inhibitor and a pharmaceutically acceptable vehicle. The pharmaceutical compositions find use in inhibiting tumor cell growth, reducing tumor size, and treating cancer in human patients. In some embodiments, the RSPO-binding agents described herein find use in the manufacture of a medicament for the treatment of cancer in combination with mitotic inhibitors. In some embodiments, the LGR-binding agents described herein find use in the manufacture of a medicament for the treatment of cancer in combination with mitotic inhibitors. In some embodiments, the mitotic inhibitors are taxanes.

Formulations are prepared for storage and use by combining a therapeutic agent with a pharmaceutically acceptable carrier, excipient, and/or stabilizer as a sterile lyophilized powder, aqueous solution, etc. (Remington: The Science and Practice of Pharmacy, 22^{nd} Edition, 2012, Pharmaceutical Press, London). Those of skill in the art generally consider pharmaceutically acceptable carriers, excipients, and/or stabilizers to be inactive ingredients of a formulation or pharmaceutical composition.

Suitable carriers, excipients, or stabilizers comprise nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (such as less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose, or sorbitol; salt-forming counter-ions such as sodium; metal complexes (e.g. Zn-protein complexes); and/or non-ionic surfactants such as polysorbate (TWEEN) or polyethylene glycol (PEG).

The therapeutic formulation can be in unit dosage form. Such formulations include tablets, pills, capsules, powders, granules, solutions or suspensions in water or non-aqueous media, or suppositories for oral, parenteral, or rectal administration or for administration by inhalation. In solid compositions such as tablets the principal active ingredient is mixed with a pharmaceutical carrier. As described herein, pharmaceutical carriers are considered to be inactive ingredients of a formulation or composition. Conventional tableting ingredients include corn starch, lactose, sucrose, sorbitol, talc, stearic acid, magnesium stearate, dicalcium phosphate or gums, and other diluents (e.g. water) to form a solid pre-formulation composition containing a homogeneous mixture of a compound, or a non-toxic pharmaceutically acceptable salt thereof. The solid pre-formulation composition is then subdivided into unit dosage forms of the type described above. The tablets, pills, etc., of the novel composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, including a number of polymeric acids and mixtures of polymeric acids with such materials as shellac, cetyl alcohol and cellulose acetate.

Pharmaceutical formulations can include a RSPO-LGR pathway inhibitor and/or a mitotic inhibitor complexed with liposomes. Liposomes can be generated by the reverse phase evaporation with a lipid composition comprising phosphatidylcholine, cholesterol, and PEG-derivatized phosphatidylethanolamine (PEG-PE). Liposomes are extruded through filters of defined pore size to yield liposomes with the desired diameter.

The RSPO-LGR pathway inhibitor and/or mitotic inhibitor can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22^{nd} Edition, 2012, Pharmaceutical Press, London.

In addition, sustained-release preparations comprising a RSPO-LGR pathway inhibitor and/or a mitotic inhibitor can be prepared. Suitable examples of sustained-release preparations include semi-permeable matrices of solid hydrophobic polymers containing the agent, which matrices are in the form of shaped articles (e.g., films or microcapsules). Examples of sustained-release matrices include polyesters, hydrogels such as poly(2-hydroxyethyl-methacrylate) or poly(vinylalcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The RSPO-LGR pathway inhibitor and mitotic inhibitor are administered as appropriate pharmaceutical compositions to a human patient according to known methods. The pharmaceutical compositions can be administered in any number of ways for either local or systemic treatment. Suitable methods of administration include, but are not limited to, intravenous (administration as a bolus or by continuous infusion over a period of time), intraarterial, intramuscular (injection or infusion), intratumoral, intraperitoneal, intracerobrospinal, subcutaneous, intra-articular, intrasynovial, intracranial (e.g., intrathecal or intraventricular), or oral. In additional, administration can be topical, (e.g., transdermal patches, ointments, lotions, creams, gels, drops, suppositories, sprays, liquids and powders) or pulmonary (e.g., by inhalation or insufflation of powders or aerosols, including by nebulizer; intratracheal, intranasal, epidermal and transdermal).

For the treatment of a disease, the appropriate dosage(s) of a RSPO-LGR pathway inhibitor in combination with a mitotic inhibitor depends on the type of disease to be treated, the severity and course of the disease, the responsiveness of the disease, whether the inhibitors are administered for therapeutic or preventative purposes, previous therapy, the patient's clinical history, and so on, all at the discretion of the treating physician. The RSPO-LGR pathway inhibitor can be administered one time or as a series of treatments spread over several days to several months, or until a cure is effected or a diminution of the disease state is achieved (e.g., reduction in tumor size). The mitotic inhibitor can be administered one time or as a series of treatments spread over several days to several months, or until a cure is effected or a diminution of the disease state is achieved (e.g., reduction in tumor size). Optimal dosing schedules for each agent can be calculated from measurements of drug accumulation in the body of the patient and will vary depending on the relative potency of an individual agent. The administering physician can determine optimum dosages, dosing methodologies, and repetition rates.

In some embodiments, combined administration includes co-administration in a single pharmaceutical formulation. In some embodiments, combined administration includes using separate formulations and consecutive administration in either order but generally within a time period such that all active agents can exert their biological activities simultaneously. In some embodiments, combined administration includes using separate formulations and a staggered dosing regimen. In some embodiments, combined administration includes using separate formulations and administration in a specific order. In some embodiments, combined administration includes using separate formulations and administration of the agents in a specific order and in a staggered dosing regimen. For example, in some embodiments, the mitotic inhibitor is administered about 1 day, about 2 days, about 3 days, about 4 days, about 5 days, about 6 days, or about 7 days after the RSPO-LGR pathway inhibitor is administered.

In certain embodiments, dosage of a RSPO-LGR pathway inhibitor is from about 0.01 μg to about 100 mg/kg of body weight, from about 0.1 μg to about 100 mg/kg of body weight, from about 1 μg to about 100 mg/kg of body weight, from about 1 mg to about 100 mg/kg of body weight, about 1 mg to about 80 mg/kg of body weight from about 10 mg to about 100 mg/kg of body weight, from about 10 mg to about 75 mg/kg of body weight, or from about 10 mg to about 50 mg/kg of body weight. In certain embodiments, the dosage of the RSPO-LGR pathway inhibitor is from about 0.1 mg to about 20 mg/kg of body weight. In some embodiments, the RSPO-LGR pathway inhibitor is administered to the subject at a dosage of about 2 mg/kg to about 10 mg/kg. In certain embodiments, the RSPO-LGR pathway inhibitor is administered once or more daily, weekly, monthly, or yearly. In certain embodiments, the RSPO-LGR pathway inhibitor is administered once every week, once every two weeks, once every three weeks, or once every four weeks.

In some embodiments of the present invention, the RSPO-LGR pathway inhibitor is administered to the subject at a dosage of about 2 mg/kg to about 20 mg/kg. In some embodiments, the RSPO-LGR pathway inhibitor or antibody is administered to the subject at a dosage of about 2 mg/kg to about 10 mg/kg. In some embodiments, the RSPO-LGR pathway inhibitor or antibody is administered to the subject at a dosage of about 2.5 mg/kg to about 10 mg/kg. In some embodiments, the RSPO-LGR pathway inhibitor or antibody is administered to the subject at a dosage of about 5 mg/kg to about 20 mg/kg.

In another embodiment, the RSPO-LGR pathway inhibitor or antibody is administered at a dosage of about 2 mg/kg to about 20 mg/kg once a week. In another embodiment, the RSPO-LGR pathway inhibitor or antibody is administered at a dosage of about 2 mg/kg to about 20 mg/kg once every two weeks. In another embodiment, the RSPO-LGR pathway inhibitor or antibody is administered at a dosage of about 2 mg/kg to about 20 mg/kg once every three weeks. In another embodiment, the RSPO-LGR pathway inhibitor or antibody is administered at a dosage of about 2 mg/kg to about 20 mg/kg once every four weeks. In another embodiment, the RSPO-LGR pathway inhibitor or antibody is administered at a dosage of about 2 mg/kg to about 5 mg/kg every three weeks. In another embodiment, the RSPO-LGR pathway inhibitor or antibody is administered at a dosage of about 3 mg/kg to about 7.5 mg/kg every four weeks.

In certain embodiments, dosage of a mitotic inhibitor is from about 20 mg/m² to about 3000 mg/m², from about 20 mg/m² to about 2000 mg/m², from about 20 mg/m² to about 1000 mg/m², from about 20 mg/m² to about 500 mg/m², or about 20 mg/m² to about 250 mg/m². In certain embodiments, the dosage of the mitotic inhibitor is from about 20 mg/m² to about 150 mg/m². In certain embodiments, the dosage of the mitotic inhibitor is about 50 mg/m². In certain embodiments, the dosage of the mitotic inhibitor is about 75 mg/m². In certain embodiments, the dosage of the mitotic inhibitor is about 90 mg/m². In certain embodiments, the dosage of the mitotic inhibitor is about 125 mg/m². In certain embodiments, the mitotic inhibitor is administered once or more daily, weekly, monthly, or yearly. In certain embodiments, the mitotic inhibitor is administered twice a day or more, once a day, once every 2 days, once every 3 days, once every 4 days, once every 5 days, once every week, once every two weeks, once every three weeks, once every four weeks, or once every week for 3 weeks of a 4 week cycle. In some embodiments, the mitotic inhibitor is administered following a dosing schedule established for a standard-of-care therapeutic agent.

In some embodiments, a RSPO-LGR pathway inhibitor and/or mitotic inhibitor can be administered at an initial higher “loading” dose, followed by one or more lower doses. In some embodiments, the frequency of administration can also change. In some embodiments, a dosing regimen can comprise administering an initial dose, followed by additional doses (or “maintenance” doses) once a week, once every two weeks, once every three weeks, or once every month. For example, a dosing regimen can comprise administering an initial loading dose, followed by a weekly maintenance dose of, for example, one-half of the initial dose. In some embodiments, a dosing regimen can comprise administering an initial loading dose, followed by maintenance doses of, for example one-half of the initial dose every other week. In some embodiments, a dosing regimen can comprise administering three initial doses for 3 weeks, followed by maintenance doses of, for example, the same amount every other week.

As is known to those of skill in the art, administration of any therapeutic agent can lead to side effects and/or toxicities. In some cases, the side effects and/or toxicities are so severe as to preclude administration of the particular agent at a therapeutically effective dose. In some cases, drug therapy must be discontinued, and other agents can be tried. However, many agents in the same therapeutic class often display similar side effects and/or toxicities, meaning that the patient either has to stop therapy, or if possible, suffer from the unpleasant side effects associated with the therapeutic agent.

Described herein are methods of treating cancer in a subject, the method comprising using a dosing strategy for administering two or more agents, which can reduce side effects and/or toxicities associated with administration of a RSPO-LGR pathway inhibitor and/or a mitotic inhibitor. In some embodiments, a method for treating cancer in a human subject comprises administering to the subject a therapeutically effective dose of a RSPO-LGR pathway inhibitor in combination with a therapeutically effective dose of a mitotic inhibitor, wherein one or both of the inhibitors are administered according to an intermittent dosing strategy. In some embodiments, the intermittent dosing strategy comprises administering an initial dose of a RSPO-LGR pathway inhibitor to the subject, and administering subsequent doses of the RSPO-LGR pathway inhibitor about once every 2 weeks. In some embodiments, the intermittent dosing strategy comprises administering an initial dose of a RSPO-LGR pathway inhibitor to the subject, and administering subsequent doses of the RSPO-LGR pathway inhibitor about once every 3 weeks. In some embodiments, the intermittent dosing strategy comprises administering an initial dose of a RSPO-LGR pathway inhibitor to the subject, and administering subsequent doses of the RSPO-LGR pathway inhibitor about once every 4 weeks. In some embodiments, the RSPO-LGR pathway inhibitor is administered using an intermittent dosing strategy and the mitotic inhibitor is administered weekly or every week for 3 weeks out of a 4 week cycle.

Combination therapy with two or more therapeutic agents often uses agents that work by different mechanisms of action, although this is not required. Combination therapy using agents with different mechanisms of action can result in additive or synergetic effects. Combination therapy can allow for a lower dose of each agent than is used in monotherapy, thereby reducing toxic side effects and/or increasing the therapeutic index of the agent(s). Combination therapy can decrease the likelihood that resistant cancer cells will develop. In some embodiments, combination therapy comprises a therapeutic agent that affects (e.g., inhibits or kills) non-tumorigenic cells and a therapeutic agent that affects (e.g., inhibits or kills) tumorigenic CSCs.

In some embodiments, the combination of a RSPO-LGR pathway inhibitor and a mitotic inhibitor results in additive or synergetic results. In some embodiments, the combination therapy results in an increase in the therapeutic index of the RSPO-LGR pathway inhibitor. In some embodiments, the combination therapy results in an increase in the therapeutic index of the mitotic inhibitor. In some embodiments, the combination therapy results in a decrease in the toxicity and/or side effects of the RSPO-LGR pathway inhibitor. In some embodiments, the combination therapy results in a decrease in the toxicity and/or side effects of the mitotic inhibitor.

The treating physician can estimate repetition rates for dosing based on measured residence times and concentrations of the drug in bodily fluids or tissues. The progress of therapy can be monitored by conventional techniques and assays.

In certain embodiments, in addition to administering a RSPO-LGR pathway inhibitor in combination with a mitotic inhibitor, treatment methods can further comprise administering at least one additional therapeutic agent prior to, concurrently with, and/or subsequently to administration of the RSPO-LGR pathway inhibitor and/or the mitotic inhibitor.

In some embodiments, the additional therapeutic agent(s) will be administered substantially simultaneously or concurrently with the RSPO-LGR pathway inhibitor or the mitotic inhibitor. For example, a subject can be given the RSPO-LGR pathway inhibitor and the mitotic inhibitor while undergoing a course of treatment with the additional therapeutic agent (e.g., additional chemotherapeutic agent). In certain embodiments, the RSPO-LGR pathway inhibitor and the mitotic inhibitor will be administered within 1 year of the treatment with the additional therapeutic agent. In certain alternative embodiments, the RSPO-LGR pathway inhibitor and the mitotic inhibitor will be administered within 10, 8, 6, 4, or 2 months of any treatment with the additional therapeutic agent. In certain other embodiments, the RSPO-LGR pathway inhibitor and the mitotic inhibitor will be administered within 4, 3, 2, or 1 week of any treatment with the additional therapeutic agent. In some embodiments, the RSPO-LGR pathway inhibitor and the mitotic inhibitor will be administered within 5, 4, 3, 2, or 1 days of any treatment with the additional therapeutic agent. It will further be appreciated that the agents or treatment can be administered to the subject within a matter of hours or minutes (i.e., substantially simultaneously) with the RSPO-LGR pathway inhibitor or the mitotic inhibitor.

Useful classes of additional therapeutic (e.g., anti-cancer) agents include, for example, auristatins, DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosureas, platinols, purine antimetabolites, puromycins, radiation sensitizers, steroids, topoisomerase inhibitors, or the like. In certain embodiments, the additional therapeutic agent is an antimetabolite, a topoisomerase inhibitor, or an angiogenesis inhibitor.

Therapeutic agents that can be administered in combination with a RSPO-LGR pathway inhibitor and a mitotic inhibitor include chemotherapeutic agents. Thus, in some embodiments, the method or treatment involves the administration of a RSPO-LGR pathway inhibitor and mitotic inhibitor in combination with a chemotherapeutic agent or cocktail of multiple different chemotherapeutic agents. Treatment with a RSPO-LGR pathway inhibitor and mitotic inhibitor can occur prior to, concurrently with, or subsequent to administration of chemotherapies. Chemotherapies contemplated include chemical substances or drugs which are known in the art and are commercially available, such as gemcitabine, irinotecan, doxorubicin, 5-fluorouracil, cytosine arabinoside (“Ara-C”), cyclophosphamide, thiotepa, busulfan, cytoxin, methotrexate, cisplatin, melphalan, and carboplatin. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in Chemotherapy Service, 1992, M. C. Perry, Editor, Williams & Wilkins, Baltimore, Md.

Chemotherapeutic agents useful in the methods described herein also include, but are not limited to, alkylating agents such as thiotepa and cyclosphosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; chlorambucil; gemcitabine; 6-thioguanine; mercaptopurine; methotrexate; platinum analogs such as cisplatin and carboplatin; platinum; etoposide (VP-16); ifosfamide; mitomycin C; mitoxantrone; novantrone; teniposide; daunomycin; aminopterin; xeloda; ibandronate; CPT11; topoisomerase inhibitor RFS 2000; difluoromethylornithine (DMFO); retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene; and antiandrogens such as flutamide, nilutamide, bicalutamide, leuprolide, and goserelin; and pharmaceutically acceptable salts, acids or derivatives of any of the above.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In some embodiments, the RSPO-LGR pathway inhibitor and mitotic inhibitor are used in combination with gemcitabine. In some embodiments, the RSPO-LGR pathway inhibitor and mitotic inhibitor are used in combination with gemcitabine for the treatment of pancreatic cancer, wherein the RSPO-LGR pathway inhibitor is OMP-131R010 and the mitotic inhibitor is paclitaxel or nab-paclitaxel (ABRAXANE).

In some embodiments, treatment can include administration of one or more cytokines (e.g., lymphokines, interleukins, tumor necrosis factors, and/or growth factors) or can be accompanied by surgical removal of tumor or cancer cells or any other therapy deemed necessary by a treating physician.

In certain embodiments, treatment involves the administration of a RSPO-LGR pathway inhibitor and a mitotic inhibitor in combination with radiation therapy. Treatment with the RSPO-LGR pathway inhibitor and the mitotic inhibitor can occur prior to, concurrently with, or subsequent to administration of radiation therapy. The dosing schedules for such radiation therapy can be determined by the skilled practitioner.

In some embodiments, Wnt pathway inhibitors can be administered in combination with a RSPO-LGR pathway inhibitor and a mitotic inhibitor. Treatment with a RSPO-LGR pathway inhibitor and mitotic inhibitor can occur prior to, concurrently with, or subsequent to administration of a Wnt pathway inhibitor. In some embodiments, a Wnt pathway inhibitor can be administered to the subject within a matter of hours or minutes (i.e., substantially simultaneously) with the RSPO-LGR pathway inhibitor or the mitotic inhibitor. Wnt pathway inhibitors have been described in, for example, U.S. Pat. Nos. 7,723,477, 8,324,361, 8,765,913, 7,982,013, 8,507,442, and U.S. Patent Publication Nos. 2013/0034551 and 2013/0045209, each of which are hereby incorporated by reference herein in their entirety for all purposes. In certain embodiments, the Wnt pathway inhibitor is an anti-Wnt antibody. In certain embodiments, the Wnt pathway inhibitor is an anti-FZD antibody. In certain embodiments, the Wnt pathway inhibitor is an anti-FZD antibody that specifically binds at least one FZD receptor selected from FZD1, FZD2, FZD3, FZD4, FZD5, FZD6, FZD7, FZD8, FZD9, and FZD10. In certain embodiments, the Wnt pathway inhibitor is an anti-FZD antibody that specifically binds at least one FZD receptor selected from FZD1, FZD2, FZD5, FZD7, and FZD8. In certain embodiments, the Wnt pathway inhibitor is vantictumab (OMP-18R5). In certain embodiments, the Wnt pathway inhibitor is a FZD-Fc soluble receptor. In certain embodiments, the Wnt pathway inhibitor is a FZD8-Fc soluble receptor. In certain embodiments, the Wnt pathway inhibitor is ipafricept (OMP-54F28).

### III. RSPO-LGR Pathway Inhibitors

Described herein are methods, including, for example, methods of inhibiting tumor growth, reducing tumor size, or treating cancer, the methods comprising administering a RSPO-LGR pathway inhibitor in combination with a mitotic inhibitor. In some embodiments, a RSPO-LGR pathway inhibitor is used in combination with a mitotic inhibitor following a sequential or staggered dosing schedule, wherein the RSPO-LGR pathway inhibitor is administered before the mitotic inhibitor.

In certain embodiments, the RSPO-LGR pathway inhibitor is an agent that binds one or more soluble extracellular components of the RSPO-LGR pathway. In certain embodiments, the RSPO-LGR pathway inhibitor is an agent that binds one or more extracellular region(s) of membrane-bound components of the RSPO-LGR pathway. In certain embodiments, the RSPO-LGR pathway inhibitor is an agent that directly modulates one or more soluble extracellular components of the RSPO-LGR pathway. In certain embodiments, the RSPO-LGR pathway inhibitor is an agent that directly modulates one or more extracellular region(s) of membrane-bound components of the RSPO-LGR pathway.

In certain embodiments, the RSPO-LGR pathway inhibitor is an agent that modulates, directly or indirectly, a component of the Wnt signaling pathway. In certain embodiments, the RSPO-LGR pathway inhibitor is an agent that inhibits β-catenin signaling. In certain embodiments, the RSPO-LGR pathway inhibitor is an agent that modulates Wnt-mediated β-catenin signaling.

In certain embodiments, the RSPO-LGR pathway inhibitor is an agent that binds one or more human RSPO proteins. These agents are referred to herein as “RSPO-binding agents”. Non-limiting examples of RSPO-binding agents can be found in U.S. Pat. Nos. 8,158,758, 8,158,757, 8,802,097, 8,088,374, and U.S. Patent Publication Nos. 2014/0017253, 2014/0134703, 2013/0337533, 2014/0186917, 2012/0263730, 2012/0039912, 2009/0220495, 2012/0088727, 2014/0056894, and 20150147333, each of which is hereby incorporated by reference herein in its entirety for all purposes.

In some embodiments, the RSPO-binding agent is an antibody. In some embodiments, the RSPO-binding agent is a polypeptide. In certain embodiments, the RSPO-binding agent binds RSPO1 (“RSPO1-binding agents”). In certain embodiments, the RSPO-binding agent binds RSPO2 (“RSPO2-binding agents”). In certain embodiments, the RSPO-binding agent binds RSPO3 (“RSPO3-binding agents”). In certain embodiments, the RSPO-binding agent specifically binds one or more human RSPO proteins. The full-length amino acid (aa) sequences for human RSPO1, RSPO2, RSPO3, and RSPO4 are known in the art and are provided herein as SEQ ID NO:1 (RSPO1), SEQ ID NO:2 (RSPO2), SEQ ID NO:3 (RSPO3), and SEQ ID NO:4 (RSPO4).

In certain embodiments, the antigen-binding site of a RSPO-binding agent (e.g., an antibody or a bispecific antibody) described herein is capable of binding (or binds) one, two, three, or four RSPOs. In certain embodiments, the antigen-binding site of a RSPO-binding agent (e.g., an antibody or a bispecific antibody) described herein is capable of binding (or binds) a first RSPO protein (e.g., RSPO1) as well as one, two, or three other RSPOs (e.g., RSPO2, RSPO3, and/or RSPO4). In some embodiments, the RSPO-binding agent (e.g., antibody) specifically binds both human RSPO and mouse RSPO.

In certain embodiments of the methods described herein, the RSPO-binding agent is an antibody that specifically binds within amino acids 21-263 of human RSPO1 (SEQ ID NO:1). In certain embodiments, the RSPO-binding agent is an antibody that specifically binds within amino acids 31-263 of human RSPO1 (SEQ ID NO:1). In certain embodiments, the RSPO-binding agent is an antibody that specifically binds within amino acids 34-135 of human RSPO1 (SEQ ID NO:1). In certain embodiments, the RSPO-binding agent is an antibody that specifically binds within amino acids 34-85 of human RSPO1 (SEQ ID NO:1). In certain embodiments, the RSPO-binding agent is an antibody that specifically binds within amino acids 91-135 of human RSPO1 (SEQ ID NO:1). In certain embodiments, the RSPO-binding agent is an antibody that specifically binds within amino acids 147-207 of human RSPO1 (SEQ ID NO:1). In certain embodiments, the RSPO-binding agent binds a furin-like cysteine-rich domain of RSPO1. In some embodiments, the RSPO-binding agent binds at least one amino acid within a furin-like cysteine-rich domain of RSPO1. In some embodiments, the RSPO-binding agent binds the thrombospondin domain of RSPO1. In some embodiments, the RSPO-binding agent binds at least one amino acid within the thrombospondin domain of RSPO1.

In certain embodiments, the RSPO-binding agent is an antibody that specifically binds within amino acids 22-243 of human RSPO2 (SEQ ID NO:2). In certain embodiments, the RSPO-binding agent is an antibody that specifically binds within amino acids 22-205 of human RSPO2 (SEQ ID NO:2). In certain embodiments, the RSPO-binding agent is an antibody that specifically binds within amino acids 35-134 of human RSPO2 (SEQ ID NO:2). In certain embodiments, the RSPO-binding agent is an antibody that specifically binds within amino acids 34-84 of human RSPO2 (SEQ ID NO:2). In certain embodiments, the RSPO-binding agent is an antibody that specifically binds within amino acids 90-134 of human RSPO2 (SEQ ID NO:2). In certain embodiments, the RSPO-binding agent binds a furin-like cysteine-rich domain of RSPO2. In some embodiments, the RSPO-binding agent binds at least one amino acid within a furin-like cysteine-rich domain of RSPO2. In some embodiments, the RSPO-binding agent binds the thrombospondin domain of RSPO2. In some embodiments, the RSPO-binding agent binds at least one amino acid within the thrombospondin domain of RSPO2.

In certain embodiments, the RSPO-binding agent is an antibody that specifically binds within amino acids 22-272 of human RSPO3 (SEQ ID NO:3). In certain embodiments, the RSPO-binding agent is an antibody that specifically binds within amino acids 22-207 of human RSPO3 (SEQ ID NO:3). In certain embodiments, the RSPO-binding agent is an antibody that specifically binds within amino acids 35-135 of human RSPO3 (SEQ ID NO:3). In certain embodiments, the RSPO-binding agent is an antibody that specifically binds within amino acids 35-86 of human RSPO3 (SEQ ID NO:3). In certain embodiments, the RSPO-binding agent is an antibody that specifically binds within amino acids 92-135 of human RSPO3 (SEQ ID NO:3). In certain embodiments, the RSPO-binding agent binds a furin-like cysteine-rich domain of RSPO3. In some embodiments, the RSPO-binding agent binds at least one amino acid within a furin-like cysteine-rich domain of RSPO3. In some embodiments, the RSPO-binding agent binds the thrombospondin domain of RSPO3. In some embodiments, the RSPO-binding agent binds at least one amino acid within the thrombospondin domain of RSPO3.

In certain embodiments, the RSPO-binding agent or antibody binds at least one RSPO protein with a dissociation constant (K_{D}) of about 1 μM or less, about 100 nM or less, about 40 nM or less, about 20 nM or less, about 10 nM or less, about 1 nM or less, or about 0.1 nM or less. In certain embodiments, a RSPO-binding agent or antibody binds at least one RSPO protein with a dissociation constant (K_{D}) of about 1 μM or less, about 100 nM or less, about 40 nM or less, about 20 nM or less, about 10 nM or less, about 1 nM or less, or about 0.1 nM or less. In some embodiments, a RSPO-binding agent or antibody binds at least one RSPO protein with a K_{D} of about 20 nM or less. In some embodiments, a RSPO-binding agent or antibody binds at least one RSPO protein with a K_{D} of about 10 nM or less. In some embodiments, a RSPO-binding agent or antibody binds at least one RSPO protein with a K_{D} of about 1 nM or less. In some embodiments, a RSPO-binding agent or antibody binds at least one RSPO protein with a K_{D} of about 0.5 nM or less. In some embodiments, a RSPO-binding agent or antibody binds at least one RSPO protein with a K_{D} of about 0.1 nM or less. In certain embodiments, a RSPO-binding agent or antibody described herein binds at least two RSPO proteins. In some embodiments, the RSPO-binding agent binds both human RSPO and mouse RSPO with a K_{D} of about 10 nM or less. In some embodiments, a RSPO-binding agent binds both human RSPO and mouse RSPO with a K_{D} of about 1 nM or less. In some embodiments, a RSPO-binding agent binds both human RSPO and mouse RSPO with a K_{D} of about 0.1 nM or less. In some embodiments, the dissociation constant of a binding agent (e.g., an antibody) to a RSPO protein is the dissociation constant determined using a RSPO fusion protein comprising at least a portion of the RSPO protein immobilized on a Biacore chip. In some embodiments, the dissociation constant of a binding agent (e.g., an antibody) to a RSPO protein is the dissociation constant determined using the binding agent captured by an anti-human IgG antibody on a Biacore chip and a RSPO protein.

In certain embodiments, the RSPO-binding agent (e.g., an antibody) binds to at least one human RSPO protein with a half maximal effective concentration (EC₅₀) of about 1 μM or less, about 100 nM or less, about 40 nM or less, about 20 nM or less, about 10 nM or less, about 1 nM or less, or about 0.1 nM or less. In certain embodiments, a RSPO-binding agent (e.g., an antibody) binds to at least one human RSPO with a half maximal effective concentration (EC₅₀) of about 1 μM or less, about 100 nM or less, about 40 nM or less, about 20 nM or less, about 10 nM or less, about 1 nM or less, or about 0.1 nM or less.

In certain embodiments, the RSPO-binding agent is a RSPO1-binding agent (e.g., an antibody) that specifically binds human RSPO1, wherein the RSPO1-binding agent (e.g., an antibody) comprises one, two, three, four, five, and/or six of the CDRs of antibody 89M5 (see Table 1).

TABLE 1

| | | |
|---|---|---|
| 89M5 | 130M23 | 131R010 |

Heavy Chain

| | | | |
|---|---|---|---|
| CDR1 | TGYTMH | SSYAMS | DYSIH |
| | (SEQ ID NO: 5) | (SEQ ID NO: 17) | (SEQ ID NO: 29) |
| CDR2 | GINPNNGGTTYNQNFKG | SISSGGSTYYPDSVKG | YIYPSNGDSGYNQKFK |
| | (SEQ ID NO: 6) | (SEQ ID NO: 18) | (SEQ ID NO: 30) |
| CDR3 | KEFSDGYYFFAY | RGGDPGVYNGDYEDAMDY | TYFANNFD |
| | (SEQ ID NO: 7) | (SEQ ID NO: 19) | (SEQ ID NO: 31) or |
| | | | ATYFANNFDY |
| | | | (SEQ ID NO: 32) |

Light Chain

| | | | |
|---|---|---|---|
| CDR1 | KASQDVIFAVA | KASQDVSSAVA | KASQSVDYDGDSYMN |
| | (SEQ ID NO: 8) | (SEQ ID NO: 20) | (SEQ ID NO: 33) |
| CDR2 | WASTRHT | WASTRHT | AASNLES |
| | (SEQ ID NO: 9) | (SEQ ID NO: 21) | (SEQ ID NO: 34) or |
| | | | AAS |
| | | | (SEQ ID NO: 35) |
| CDR3 | QQHYSTPW | QQHYSTP | QQSNEDPLT |
| | (SEQ ID NO: 10) | (SEQ ID NO: 22) | (SEQ ID NO: 36) or |
| | | | QQSNEDPLTF |
| | | | (SEQ ID NO: 37) |

In certain embodiments, the RSPO-binding agent is a RSPO1-binding agent (e.g., an antibody) that specifically binds human RSPO1, wherein the RSPO1-binding agent comprises a heavy chain CDR1 comprising TGYTMH (SEQ ID NO:5), a heavy chain CDR2 comprising GINPNNGGTTYNQNFKG (SEQ ID NO:6), and a heavy chain CDR3 comprising KEFSDGYYFFAY (SEQ ID NO:7). In some embodiments, the RSPO1-binding agent further comprises a light chain CDR1 comprising KASQDVIFAVA (SEQ ID NO:8), a light chain CDR2 comprising WASTRHT (SEQ ID NO:9), and a light chain CDR3 comprising QQHYSTPW (SEQ ID NO:10). In some embodiments, the RSPO1-binding agent comprises a light chain CDR1 comprising KASQDVIFAVA (SEQ ID NO:8), a light chain CDR2 comprising WASTRHT (SEQ ID NO:9), and a light chain CDR3 comprising QQHYSTPW (SEQ ID NO:10). In certain embodiments, the RSPO1-binding agent comprises: (a) a heavy chain CDR1 comprising TGYTMH (SEQ ID NO:5), a heavy chain CDR2 comprising GINPNNGGTTYNQNFKG (SEQ ID NO:6), and a heavy chain CDR3 comprising KEFSDGYYFFAY (SEQ ID NO:7); and (b) a light chain CDR1 comprising KASQDVIFAVA (SEQ ID NO:8), a light chain CDR2 comprising WASTRHT (SEQ ID NO:9), and a light chain CDR3 comprising QQHYSTPW (SEQ ID NO:10).

In certain embodiments, the RSPO-binding agent is a RSPO1-binding agent (e.g., an antibody or bispecific antibody) that specifically binds human RSPO1, wherein the RSPO1-binding agent comprises: (a) a heavy chain CDR1 comprising TGYTMH (SEQ ID NO:5) or a variant thereof comprising 1, 2, 3, or 4 amino acid substitutions; (b) a heavy chain CDR2 comprising GINPNNGGTTYNQNFKG (SEQ ID NO:6) or a variant thereof comprising 1, 2, 3, or 4 amino acid substitutions; (c) a heavy chain CDR3 comprising KEFSDGYYFFAY (SEQ ID NO:7) or a variant thereof comprising 1, 2, 3, or 4 amino acid substitutions; (d) a light chain CDR1 comprising KASQDVIFAVA (SEQ ID NO:8) or a variant thereof comprising 1, 2, 3, or 4 amino acid substitutions; (e) a light chain CDR2 comprising WASTRHT (SEQ ID NO:9) or a variant thereof comprising 1, 2, 3, or 4 amino acid substitutions; and (f) a light chain CDR3 comprising QQHYSTPW (SEQ ID NO:10) or a variant thereof comprising 1, 2, 3, or 4 amino acid substitutions. In certain embodiments, the amino acid substitutions are conservative substitutions. In some embodiments, the substitutions are made as part of a germline humanization process.

In certain embodiments, the RSPO-binding agent is a RSPO1-binding agent (e.g., an antibody) that specifically binds RSPO1, wherein the RSPO1-binding agent comprises a heavy chain variable region having at least about 80% sequence identity to SEQ ID NO:11 and/or a light chain variable region having at least 80% sequence identity to SEQ ID NO:12. In certain embodiments, the RSPO1-binding agent comprises a heavy chain variable region having at least about 85%, at least about 90%, at least about 95%, at least about 97%, or at least about 99% sequence identity to SEQ ID NO:11. In certain embodiments, the RSPO1-binding agent comprises a light chain variable region having at least about 85%, at least about 90%, at least about 95%, at least about 97%, or at least about 99% sequence identity to SEQ ID NO:12. In certain embodiments, the RSPO1-binding agent comprises a heavy chain variable region having at least about 95% sequence identity to SEQ ID NO:11 and/or a light chain variable region having at least about 95% sequence identity to SEQ ID NO:12. In certain embodiments, the RSPO1-binding agent comprises a heavy chain variable region comprising SEQ ID NO:11 and/or a light chain variable region comprising SEQ ID NO:12. In certain embodiments, the RSPO1-binding agent comprises a heavy chain variable region comprising SEQ ID NO:11 and a light chain variable region comprising SEQ ID NO:12. In certain embodiments, the RSPO1-binding agent comprises a heavy chain variable region consisting of SEQ ID NO:11 and a light chain variable region consisting of SEQ ID NO:12.

In certain embodiments, the RSPO-binding agent is a RSPO1-binding agent (e.g., an antibody) that specifically binds RSPO1, wherein the RSPO1-binding agent comprises a heavy chain variable region having at least about 80% sequence identity to SEQ ID NO:44 and/or a light chain variable region having at least 80% sequence identity to SEQ ID NO:45. In certain embodiments, the RSPO1-binding agent comprises a heavy chain variable region having at least about 85%, at least about 90%, at least about 95%, at least about 97%, or at least about 99% sequence identity to SEQ ID NO:44. In certain embodiments, the RSPO1-binding agent comprises a light chain variable region having at least about 85%, at least about 90%, at least about 95%, at least about 97%, or at least about 99% sequence identity to SEQ ID NO:45. In certain embodiments, the RSPO1-binding agent comprises a heavy chain variable region having at least about 95% sequence identity to SEQ ID NO:44 and/or a light chain variable region having at least about 95% sequence identity to SEQ ID NO:45. In certain embodiments, the RSPO1-binding agent comprises a heavy chain variable region comprising SEQ ID NO:44 and/or a light chain variable region comprising SEQ ID NO:45. In certain embodiments, the RSPO1-binding agent comprises a heavy chain variable region comprising SEQ ID NO:44 and a light chain variable region comprising SEQ ID NO:45. In certain embodiments, the RSPO1-binding agent comprises a heavy chain variable region consisting of SEQ ID NO:44 and a light chain variable region consisting of SEQ ID NO:45.

In certain embodiments, the RSPO-binding agent is a RSPO1-binding agent (e.g., an antibody) that specifically binds RSPO1, wherein the RSPO1-binding agent comprises: (a) a heavy chain having at least 90% sequence identity to SEQ ID NO:13 or SEQ ID NO:14; and/or (b) a light chain having at least 90% sequence identity to SEQ ID NO:15 or SEQ ID NO:16. In some embodiments, the RSPO1-binding agent comprises: (a) a heavy chain having at least 95% sequence identity to SEQ ID NO:13 or SEQ ID NO:14; and/or (b) a light chain having at least 95% sequence identity to SEQ ID NO:15 or SEQ ID NO:16. In some embodiments, the RSPO1-binding agent comprises a heavy chain comprising SEQ ID NO:14 and/or a light chain comprising SEQ ID NO:16. In some embodiments, the RSPO1-binding agent comprises a heavy chain comprising SEQ ID NO:14 and a light chain comprising SEQ ID NO:16.

In certain embodiments, the RSPO-binding agent is a RSPO1-binding agent (e.g., an antibody) that specifically binds RSPO1, wherein the RSPO1-binding agent comprises: (a) a heavy chain having at least 90% sequence identity to SEQ ID NO:46 or SEQ ID NO:47; and/or (b) a light chain having at least 90% sequence identity to SEQ ID NO:48 or SEQ ID NO:49. In some embodiments, the RSPO1-binding agent comprises: (a) a heavy chain having at least 95% sequence identity to SEQ ID NO:46 or SEQ ID NO:47; and/or (b) a light chain having at least 95% sequence identity to SEQ ID NO:48 or SEQ ID NO:49. In some embodiments, the RSPO1-binding agent comprises a heavy chain comprising SEQ ID NO:47 and/or a light chain comprising SEQ ID NO:49. In some embodiments, the RSPO1-binding agent comprises a heavy chain comprising SEQ ID NO:47 and a light chain comprising SEQ ID NO:49.

In certain embodiments, a RSPO1-binding agent comprises the heavy chain variable region and light chain variable region of antibody h89M5-H8L5. In certain embodiments, a RSPO1-binding agent comprises the heavy chain and light chain of antibody h89M5-H8L5 (with or without the leader sequence). In certain embodiments, a RSPO1-binding agent is antibody h89M5-H8L5. In certain embodiments, a RSPO1-binding agent comprises the heavy chain variable region and/or light chain variable region of antibody h89M5-H8L5 in a chimeric form of the antibody. In some embodiments, the anti-RSPO1 antibody is h89M5-H8L5.

In certain embodiments, a RSPO1-binding agent comprises the heavy chain variable region and light chain variable region of antibody h89M5-H2L2. In certain embodiments, a RSPO1-binding agent comprises the heavy chain and light chain of antibody h89M5-H2L2 (with or without the leader sequence). In certain embodiments, a RSPO1-binding agent is antibody h89M5-H2L2. In certain embodiments, a RSPO1-binding agent comprises the heavy chain variable region and/or light chain variable region of antibody h89M5-H2L2 in a chimeric form of the antibody. In some embodiments, the anti-RSPO1 antibody is h89M5-H2L2.

In certain embodiments, a RSPO1-binding agent comprises the heavy chain CDRs and/or light chain CDRs of antibody 89M5. The hybridoma cell line producing the 89M5 antibody was deposited with American Type Culture Collection (ATCC), 10801 University Boulevard, Manassas, Va., USA, under the conditions of the Budapest Treaty on Jun. 30, 2011 and assigned ATCC deposit designation number PTA-11970.

Plasmids encoding the heavy chain and light chain of antibody h89M5-H8L5 were deposited with ATCC, 10801 University Boulevard, Manassas, Va., USA, under the conditions of the Budapest Treaty on Aug. 15, 2014 and assigned ATCC deposit designation number PTA-121494 and PTA-121495. In some embodiments, a RSPO1-binding agent comprises a heavy chain variable region encoded by the plasmid deposited with ATCC and designated PTA-121494. In some embodiments, a RSPO1-binding agent comprises a light chain variable region encoded by the plasmid deposited with ATCC and designated PTA-121495. In some embodiments, a RSPO1-binding agent comprises a heavy chain variable region encoded by the plasmid deposited with ATCC and designated PTA-121494 and a light chain variable region encoded by the plasmid deposited with ATCC and designated PTA-121495. In some embodiments, a RSPO1-binding agent comprises a heavy chain encoded by the plasmid deposited with ATCC and designated PTA-121494. In some embodiments, a RSPO1-binding agent comprises a light chain encoded by the plasmid deposited with ATCC and designated PTA-121495. In some embodiments, a RSPO1-binding agent comprises a heavy chain encoded by the plasmid deposited with ATCC and designated PTA-121494 and a light chain encoded by the plasmid deposited with ATCC and designated PTA-121495.

In certain embodiments, a RSPO1-binding agent comprises, consists essentially of, or consists of, antibody h89M5-H8L5. In certain embodiments, a RSPO1-binding agent comprises, consists essentially of, or consists of, a variant of antibody 89M5. In certain embodiments, a RSPO1-binding agent comprises, consists essentially of, or consists of, a variant of antibody h89M5-H8L5.

In certain embodiments, a RSPO1-binding agent comprises, consists essentially of, or consists of, antibody h89M5-H2L2. In certain embodiments, a RSPO1-binding agent comprises, consists essentially of, or consists of, a variant of antibody 89M5. In certain embodiments, a RSPO1-binding agent comprises, consists essentially of, or consists of, a variant of antibody h89M5-H2L2.

In certain embodiments of the methods described herein, the RSPO-binding agent is a RSPO2-binding agent (e.g., an antibody) that specifically binds human RSPO2, wherein the RSPO2-binding agent (e.g., an antibody) comprises one, two, three, four, five, and/or six of the CDRs of antibody 130M23 (see Table 1).

In certain embodiments, the RSPO-binding agent is a RSPO2-binding agent (e.g., an antibody) that specifically binds human RSPO2, wherein the RSPO2-binding agent comprises a heavy chain CDR1 comprising SSYAMS (SEQ ID NO:17), a heavy chain CDR2 comprising SISSGGSTYYPDSVKG (SEQ ID NO:18), and a heavy chain CDR3 comprising RGGDPGVYNGDYEDAMDY (SEQ ID NO:19). In some embodiments, the RSPO2-binding agent further comprises a light chain CDR1 comprising KASQDVSSAVA (SEQ ID NO:20), a light chain CDR2 comprising WASTRHT (SEQ ID NO:21), and a light chain CDR3 comprising QQHYSTP (SEQ ID NO:22). In some embodiments, the RSPO2-binding agent comprises a light chain CDR1 comprising KASQDVSSAVA (SEQ ID NO:20), a light chain CDR2 comprising WASTRHT (SEQ ID NO:21), and a light chain CDR3 comprising QQHYSTP (SEQ ID NO:22). In certain embodiments, the RSPO2-binding agent comprises: (a) a heavy chain CDR1 comprising SSYAMS (SEQ ID NO:17), a heavy chain CDR2 comprising SISSGGSTYYPDSVKG (SEQ ID NO:18), and a heavy chain CDR3 comprising RGGDPGVYNGDYEDAMDY (SEQ ID NO:19); and (b) a light chain CDR1 comprising KASQDVSSAVA (SEQ ID NO:20), a light chain CDR2 comprising WASTRHT (SEQ ID NO:21), and a light chain CDR3 comprising QQHYSTP (SEQ ID NO:22).

In certain embodiments, the RSPO-binding agent is a RSPO2-binding agent (e.g., an antibody or bispecific antibody) that specifically binds human RSPO2, wherein the RSPO2-binding agent comprises: (a) a heavy chain CDR1 comprising SSYAMS (SEQ ID NO:17) or a variant thereof comprising 1, 2, 3, or 4 amino acid substitutions; (b) a heavy chain CDR2 comprising SISSGGSTYYPDSVKG (SEQ ID NO:18) or a variant thereof comprising 1, 2, 3, or 4 amino acid substitutions; (c) a heavy chain CDR3 comprising RGGDPGVYNGDYEDAMDY (SEQ ID NO:19) or a variant thereof comprising 1, 2, 3, or 4 amino acid substitutions; (d) a light chain CDR1 comprising KASQDVSSAVA (SEQ ID NO:20) or a variant thereof comprising 1, 2, 3, or 4 amino acid substitutions; (e) a light chain CDR2 comprising WASTRHT (SEQ ID NO:21) or a variant thereof comprising 1, 2, 3, or 4 amino acid substitutions; and (f) a light chain CDR3 comprising QQHYSTP (SEQ ID NO:22) or a variant thereof comprising 1, 2, 3, or 4 amino acid substitutions. In certain embodiments, the amino acid substitutions are conservative substitutions. In some embodiments, the substitutions are made as part of a germline humanization process.

In certain embodiments, the RSPO-binding agent is a RSPO2-binding agent (e.g., an antibody) that specifically binds RSPO2, wherein the RSPO2-binding agent comprises a heavy chain variable region having at least about 80% sequence identity to SEQ ID NO:23 and/or a light chain variable region having at least 80% sequence identity to SEQ ID NO:24 or SEQ ID NO:50. In certain embodiments, the RSPO2-binding agent comprises a heavy chain variable region having at least about 85%, at least about 90%, at least about 95%, at least about 97%, or at least about 99% sequence identity to SEQ ID NO:23. In certain embodiments, the RSPO2-binding agent comprises a light chain variable region having at least about 85%, at least about 90%, at least about 95%, at least about 97%, or at least about 99% sequence identity to SEQ ID NO:24. In certain embodiments, the RSPO2-binding agent comprises a light chain variable region having at least about 85%, at least about 90%, at least about 95%, at least about 97%, or at least about 99% sequence identity to SEQ ID NO:50. In certain embodiments, the RSPO2-binding agent comprises a heavy chain variable region having at least about 95% sequence identity to SEQ ID NO:23 and/or a light chain variable region having at least about 95% sequence identity to SEQ ID NO:24 or SEQ ID NO:50. In certain embodiments, the RSPO2-binding agent comprises a heavy chain variable region comprising SEQ ID NO:23 and/or a light chain variable region comprising SEQ ID NO:24 or SEQ ID NO:50. In certain embodiments, the RSPO2-binding agent comprises a heavy chain variable region comprising SEQ ID NO:23 and a light chain variable region comprising SEQ ID NO:24. In certain embodiments, the RSPO2-binding agent comprises a heavy chain variable region comprising SEQ ID NO:23 and a light chain variable region comprising SEQ ID NO:50. In certain embodiments, the RSPO2-binding agent comprises a heavy chain variable region consisting of SEQ ID NO:23 and a light chain variable region consisting of SEQ ID NO:24. In certain embodiments, the RSPO2-binding agent comprises a heavy chain variable region consisting of SEQ ID NO:23 and a light chain variable region consisting of SEQ ID NO:50.

In certain embodiments, the RSPO-binding agent is a RSPO2-binding agent (e.g., an antibody) that specifically binds RSPO2, wherein the RSPO2-binding agent comprises: (a) a heavy chain having at least 90% sequence identity to SEQ ID NO:25 or SEQ ID NO:26; and/or (b) a light chain having at least 90% sequence identity to SEQ ID NO:27 or SEQ ID NO:28. In some embodiments, the RSPO2-binding agent comprises: (a) a heavy chain having at least 95% sequence identity to SEQ ID NO:25 or SEQ ID NO:26; and/or (b) a light chain having at least 95% sequence identity to SEQ ID NO:27 or SEQ ID NO:28. In some embodiments, the RSPO2-binding agent comprises a heavy chain comprising SEQ ID NO:26 and/or a light chain comprising SEQ ID NO:28. In some embodiments, the RSPO2-binding agent comprises a heavy chain comprising SEQ ID NO:26 and a light chain comprising SEQ ID NO:28.

In certain embodiments, the RSPO-binding agent is a RSPO2-binding agent (e.g., an antibody) that specifically binds RSPO2, wherein the RSPO2-binding agent comprises: (a) a heavy chain having at least 90% sequence identity to SEQ ID NO:25 or SEQ ID NO:26; and/or (b) a light chain having at least 90% sequence identity to SEQ ID NO:51 or SEQ ID NO:52. In some embodiments, the RSPO2-binding agent comprises: (a) a heavy chain having at least 95% sequence identity to SEQ ID NO:25 or SEQ ID NO:26; and/or (b) a light chain having at least 95% sequence identity to SEQ ID NO:51 or SEQ ID NO:52. In some embodiments, the RSPO2-binding agent comprises a heavy chain comprising SEQ ID NO:26 and/or a light chain comprising SEQ ID NO:52. In some embodiments, the RSPO2-binding agent comprises a heavy chain comprising SEQ ID NO:26 and a light chain comprising SEQ ID NO:52.

In certain embodiments, a RSPO2-binding agent comprises the heavy chain variable region and light chain variable region of antibody h130M23-H1L6. In certain embodiments, a RSPO2-binding agent comprises the heavy chain and light chain of antibody h130M23-H1L6 (with or without the leader sequence). In certain embodiments, a RSPO2-binding agent is antibody h130M23-H1L6. In certain embodiments, a RSPO2-binding agent comprises the heavy chain variable region and/or light chain variable region of antibody h130M23-H1L6 in a chimeric form of the antibody. In some embodiments, the anti-RSPO2 antibody is h130M23-H1L6.

In certain embodiments, a RSPO2-binding agent comprises the heavy chain variable region and light chain variable region of antibody h130M23-H1L2. In certain embodiments, a RSPO2-binding agent comprises the heavy chain and light chain of antibody h130M23-H1L2 (with or without the leader sequence). In certain embodiments, a RSPO2-binding agent is antibody h130M23-H1L2. In certain embodiments, a RSPO2-binding agent comprises the heavy chain variable region and/or light chain variable region of antibody h130M23-H1L2 in a chimeric form of the antibody. In some embodiments, the anti-RSPO2 antibody is h130M23-H1L2.

In certain embodiments of the methods described herein, a RSPO2-binding agent comprises the heavy chain CDRs and/or light chain CDRs of antibody 130M23. The hybridoma cell line producing the 130M23 antibody was deposited with ATCC, 10801 University Boulevard, Manassas, Va., USA, under the conditions of the Budapest Treaty on Aug. 10, 2011 and assigned ATCC deposit designation number PTA-12021.

In certain embodiments, a RSPO2-binding agent comprises, consists essentially of, or consists of, antibody h130M23-H1L6. In certain embodiments, a RSPO2-binding agent comprises, consists essentially of, or consists of, a variant of antibody 130M23. In certain embodiments, a RSPO2-binding agent comprises, consists essentially of, or consists of, a variant of antibody h130M23-H1L6.

In certain embodiments, a RSPO2-binding agent comprises, consists essentially of, or consists of, antibody h130M23-H1L2. In certain embodiments, a RSPO2-binding agent comprises, consists essentially of, or consists of, a variant of antibody 130M23. In certain embodiments, a RSPO2-binding agent comprises, consists essentially of, or consists of, a variant of antibody h130M23-H1L2.

In certain embodiments of the methods described herein, the RSPO-binding agent is a RSPO3-binding agent (e.g., an antibody) that specifically binds human RSPO3, wherein the RSPO3-binding agent (e.g., an antibody) comprises one, two, three, four, five, and/or six of the CDRs of antibody 131R010 (see Table 1 herein).

In certain embodiments, the RSPO-binding agent is a RSPO3-binding agent (e.g., an antibody) that specifically binds human RSPO3, wherein the RSPO3-binding agent comprises a heavy chain CDR1 comprising DYSIH (SEQ ID NO:29), a heavy chain CDR2 comprising YIYPSNGDSGYNQKFK (SEQ ID NO:30), and a heavy chain CDR3 comprising TYFANNFD (SEQ ID NO:31) or ATYFANNFDY (SEQ ID NO:32). In some embodiments, the RSPO3-binding agent further comprises a light chain CDR1 comprising KASQSVDYDGDSYMN (SEQ ID NO:33), a light chain CDR2 comprising AASNLES (SEQ ID NO:34) or AAS (SEQ ID NO:35), and a light chain CDR3 comprising QQSNEDPLT (SEQ ID NO:36) or QQSNEDPLTF (SEQ ID NO:37). In some embodiments, the RSPO3-binding agent comprises a light chain CDR1 comprising KASQSVDYDGDSYMN (SEQ ID NO:33), a light chain CDR2 comprising AASNLES (SEQ ID NO:34) or AAS (SEQ ID NO:35), and a light chain CDR3 comprising QQSNEDPLT (SEQ ID NO:36) or QQSNEDPLTF (SEQ ID NO:37). In certain embodiments, the RSPO3-binding agent comprises: (a) a heavy chain CDR1 comprising DYSIH (SEQ ID NO:29), a heavy chain CDR2 comprising YIYPSNGDSGYNQKFK (SEQ ID NO:30), and a heavy chain CDR3 comprising TYFANNFD (SEQ ID NO:31); and (b) a light chain CDR1 comprising KASQSVDYDGDSYMN (SEQ ID NO:33), a light chain CDR2 comprising AASNLES (SEQ ID NO:34), and a light chain CDR3 comprising QQSNEDPLT (SEQ ID NO:36). In certain embodiments, the RSPO3-binding agent comprises: (a) a heavy chain CDR1 comprising DYSIH (SEQ ID NO:29), a heavy chain CDR2 comprising YIYPSNGDSGYNQKFK (SEQ ID NO:30), and a heavy chain CDR3 comprising ATYFANNFDY (SEQ ID NO:32); and (b) a light chain CDR1 comprising KASQSVDYDGDSYMN (SEQ ID NO:33), a light chain CDR2 comprising AASNLES (SEQ ID NO:34), and a light chain CDR3 comprising QQSNEDPLT (SEQ ID NO:36).

In certain embodiments, the RSPO-binding agent is a RSPO3-binding agent (e.g., an antibody or bispecific antibody) that specifically binds human RSPO3, wherein the RSPO3-binding agent comprises: (a) a heavy chain CDR1 comprising DYSIH (SEQ ID NO:29) or a variant thereof comprising 1, 2, 3, or 4 amino acid substitutions; (b) a heavy chain CDR2 comprising YIYPSNGDSGYNQKFK (SEQ ID NO:30) or a variant thereof comprising 1, 2, 3, or 4 amino acid substitutions; (c) a heavy chain CDR3 comprising TYFANNFD (SEQ ID NO:31), ATYFANNFDY (SEQ ID NO:32), or a variant thereof comprising 1, 2, 3, or 4 amino acid substitutions; (d) a light chain CDR1 comprising KASQSVDYDGDSYMN (SEQ ID NO:33) or a variant thereof comprising 1, 2, 3, or 4 amino acid substitutions; (e) a light chain CDR2 comprising AASNLES (SEQ ID NO:34), AAS (SEQ ID NO:35), or a variant thereof comprising 1, 2, 3, or 4 amino acid substitutions; and (f) a light chain CDR3 comprising QQSNEDPLT (SEQ ID NO:36), QQSNEDPLTF (SEQ ID NO:37), or a variant thereof comprising 1, 2, 3, or 4 amino acid substitutions. In certain embodiments, the amino acid substitutions are conservative substitutions. In some embodiments, the substitutions are made as part of a germline humanization process.

In certain embodiments, the RSPO-binding agent is a RSPO3-binding agent (e.g., an antibody) that specifically binds RSPO3, wherein the RSPO3-binding agent comprises a heavy chain variable region having at least about 80% sequence identity to SEQ ID NO:38 and/or a light chain variable region having at least 80% sequence identity to SEQ ID NO:39. In certain embodiments, the RSPO3-binding agent comprises a heavy chain variable region having at least about 85%, at least about 90%, at least about 95%, at least about 97%, or at least about 99% sequence identity to SEQ ID NO:38. In certain embodiments, the RSPO3-binding agent comprises a light chain variable region having at least about 85%, at least about 90%, at least about 95%, at least about 97%, or at least about 99% sequence identity to SEQ ID NO:39. In certain embodiments, the RSPO3-binding agent comprises a heavy chain variable region having at least about 95% sequence identity to SEQ ID NO:38 and/or a light chain variable region having at least about 95% sequence identity to SEQ ID NO:39. In certain embodiments, the RSPO3-binding agent comprises a heavy chain variable region comprising SEQ ID NO:38 and/or a light chain variable region comprising SEQ ID NO:39. In certain embodiments, the RSPO3-binding agent comprises a heavy chain variable region comprising SEQ ID NO:38 and a light chain variable region comprising SEQ ID NO:39. In certain embodiments, the RSPO3-binding agent comprises a heavy chain variable region consisting of SEQ ID NO:38 and a light chain variable region consisting of SEQ ID NO:39.

In certain embodiments, the RSPO-binding agent is a RSPO3-binding agent (e.g., an antibody) that specifically binds RSPO3, wherein the RSPO3-binding agent comprises: (a) a heavy chain having at least 90% sequence identity to SEQ ID NO:40 or SEQ ID NO:41; and/or (b) a light chain having at least 90% sequence identity to SEQ ID NO:42 or SEQ ID NO:43. In some embodiments, the RSPO3-binding agent comprises: (a) a heavy chain having at least 95% sequence identity to SEQ ID NO:40 or SEQ ID NO:41; and/or (b) a light chain having at least 95% sequence identity to SEQ ID NO:42 or SEQ ID NO:43. In some embodiments, the RSPO3-binding agent comprises a heavy chain comprising SEQ ID NO:41 and/or a light chain comprising SEQ ID NO:43. In some embodiments, the RSPO3-binding agent comprises a heavy chain comprising SEQ ID NO:41 and a light chain comprising SEQ ID NO:43.

In certain embodiments, a RSPO3-binding agent comprises the heavy chain variable region and light chain variable region of antibody 131R010. In certain embodiments, a RSPO3-binding agent comprises the heavy chain and light chain of antibody 131R010 (with or without the leader sequence). In certain embodiments, a RSPO3-binding agent is antibody 131R010. In certain embodiments, a RSPO3-binding agent comprises the heavy chain variable region and/or light chain variable region of antibody 131R010 in a chimeric form of the antibody. In certain embodiments, a RSPO3-binding agent comprises the heavy chain CDRs and/or light chain CDRs of antibody 131R010. In some embodiments, the anti-RSPO3 antibody is 131R010.

Plasmids encoding the heavy chain and light chain of antibody 131R010 were deposited with the American Type Culture Collection (ATCC), 10801 University Boulevard, Manassas, Va., USA, under the conditions of the Budapest Treaty on Jun. 18, 2013 and assigned ATCC deposit designation number PTA-120420 and PTA-120421. In some embodiments, the RSPO3-binding agent comprises a heavy chain variable region encoded by the plasmid deposited with ATCC and designated PTA-120420. In some embodiments, the RSPO3-binding agent comprises a light chain variable region encoded by the plasmid deposited with ATCC and designated PTA-120421. In some embodiments, the RSPO3-binding agent comprises a heavy chain variable region encoded by the plasmid deposited with ATCC and designated PTA-120420 and a light chain variable region encoded by the plasmid deposited with ATCC and designated PTA-120421. In some embodiments, the RSPO3-binding agent comprises a heavy chain encoded by the plasmid deposited with ATCC and designated PTA-120420. In some embodiments, the RSPO3-binding agent comprises a light chain encoded by the plasmid deposited with ATCC and designated PTA-120421. In some embodiments, the RSPO3-binding agent comprises a heavy chain encoded by the plasmid deposited with ATCC and designated PTA-120420 and a light chain encoded by the plasmid deposited with ATCC and designated PTA-120421.

In certain embodiments, a RSPO3-binding agent comprises, consists essentially of, or consists of, antibody 131R010. In certain embodiments, a RSPO3-binding agent comprises, consists essentially of, or consists of, a variant of antibody 131R010.

Described herein are methods comprising polypeptides, including, but not limited to, antibodies that specifically bind at least one human RSPO protein. In some embodiments, a polypeptide binds human RSPO1. In some embodiments, a polypeptide binds human RSPO2. In some embodiments, a polypeptide binds human RSPO3.

In certain embodiments, the polypeptide comprises one, two, three, four, five, and/or six of the CDRs of antibody 89M5 (see Table 1 herein). In certain embodiments, the polypeptide comprises one, two, three, four, five, and/or six of the CDRs of antibody 130M23 (see Table 1 herein). In certain embodiments, the polypeptide comprises one, two, three, four, five, and/or six of the CDRs of antibody 131R010 (see Table 1 herein). In some embodiments, the polypeptide comprises CDRs with up to four (i.e., 0, 1, 2, 3, or 4) amino acid substitutions per CDR. In certain embodiments, the heavy chain CDR(s) are contained within a heavy chain variable region. In certain embodiments, the light chain CDR(s) are contained within a light chain variable region.

In some embodiments, the RSPO-binding agent is a polypeptide that specifically binds a human RSPO1, wherein the polypeptide comprises an amino acid sequence having at least about 80% sequence identity to SEQ ID NO:11 and/or SEQ ID NO:12. In some embodiments, the polypeptide comprises an amino acid sequence having at least about 80% sequence identity to SEQ ID NO:13 and/or an amino acid sequence having at least about 80% sequence identity to SEQ ID NO:15. In some embodiments, the polypeptide comprises an amino acid sequence having at least about 80% sequence identity to SEQ ID NO:14 and/or an amino acid sequence having at least about 80% sequence identity to SEQ ID NO:16. In certain embodiments, the polypeptide comprises an amino acid sequence having at least about 85%, at least about 90%, at least about 95%, at least about 97%, or at least about 99% sequence identity to SEQ ID NO:11, SEQ ID NO:12, SEQ ID NO:13, SEQ ID NO:14, SEQ ID NO:15, or SEQ ID NO:16. In certain embodiments, the polypeptide comprises an amino acid sequence having at least about 95% sequence identity to SEQ ID NO:11 and/or an amino acid sequence having at least about 95% sequence identity to SEQ ID NO:12. In certain embodiments, the polypeptide comprises an amino acid sequence having at least about 95% sequence identity to SEQ ID NO:13 and/or an amino acid sequence having at least about 95% sequence identity to SEQ ID NO:15. In certain embodiments, the polypeptide comprises an amino acid sequence having at least about 95% sequence identity to SEQ ID NO:14 and/or an amino acid sequence having at least about 95% sequence identity to SEQ ID NO:16. In certain embodiments, the polypeptide comprises an amino acid sequence of SEQ ID NO:11 and/or an amino acid sequence of SEQ ID NO:12. In certain embodiments, the polypeptide comprises an amino acid sequence of SEQ ID NO:13 and/or an amino acid sequence of SEQ ID NO:15. In certain embodiments, the polypeptide comprises an amino acid sequence of SEQ ID NO:14 and/or an amino acid sequence of SEQ ID NO:16.

In some embodiments, the RSPO-binding agent is a polypeptide that specifically binds a human RSPO1, wherein the polypeptide comprises an amino acid sequence having at least about 80% sequence identity to SEQ ID NO:44 and/or SEQ ID NO:45. In some embodiments, the polypeptide comprises an amino acid sequence having at least about 80% sequence identity to SEQ ID NO:46 and/or an amino acid sequence having at least about 80% sequence identity to SEQ ID NO:48. In some embodiments, the polypeptide comprises an amino acid sequence having at least about 80% sequence identity to SEQ ID NO:47 and/or an amino acid sequence having at least about 80% sequence identity to SEQ ID NO:49. In certain embodiments, the polypeptide comprises an amino acid sequence having at least about 85%, at least about 90%, at least about 95%, at least about 97%, or at least about 99% sequence identity to SEQ ID NO:44, SEQ ID NO:45, SEQ ID NO:46, SEQ ID NO:47, SEQ ID NO:48, or SEQ ID NO:49. In certain embodiments, the polypeptide comprises an amino acid sequence having at least about 95% sequence identity to SEQ ID NO:44 and/or an amino acid sequence having at least about 95% sequence identity to SEQ ID NO:45. In certain embodiments, the polypeptide comprises an amino acid sequence having at least about 95% sequence identity to SEQ ID NO:46 and/or an amino acid sequence having at least about 95% sequence identity to SEQ ID NO:48. In certain embodiments, the polypeptide comprises an amino acid sequence having at least about 95% sequence identity to SEQ ID NO:47 and/or an amino acid sequence having at least about 95% sequence identity to SEQ ID NO:49. In certain embodiments, the polypeptide comprises an amino acid sequence of SEQ ID NO:44 and/or an amino acid sequence of SEQ ID NO:45. In certain embodiments, the polypeptide comprises an amino acid sequence of SEQ ID NO:46 and/or an amino acid sequence of SEQ ID NO:48. In certain embodiments, the polypeptide comprises an amino acid sequence of SEQ ID NO:47 and/or an amino acid sequence of SEQ ID NO:49.

In some embodiments, the RSPO-binding agent is a polypeptide that specifically binds a human RSPO2, wherein the polypeptide comprises an amino acid sequence having at least about 80% sequence identity to SEQ ID NO:23 and/or SEQ ID NO:24. In some embodiments, the RSPO-binding agent is a polypeptide that specifically binds a human RSPO2, wherein the polypeptide comprises an amino acid sequence having at least about 80% sequence identity to SEQ ID NO:23 and/or SEQ ID NO:50. In some embodiments, the polypeptide comprises an amino acid sequence having at least about 80% sequence identity to SEQ ID NO:25 and/or an amino acid sequence having at least about 80% sequence identity to SEQ ID NO:27 or SEQ ID NO:51. In some embodiments, the polypeptide comprises an amino acid sequence having at least about 80% sequence identity to SEQ ID NO:26 and/or an amino acid sequence having at least about 80% sequence identity to SEQ ID NO:28 or SEQ ID NO:52. In certain embodiments, the polypeptide comprises an amino acid sequence having at least about 85%, at least about 90%, at least about 95%, at least about 97%, or at least about 99% sequence identity to SEQ ID NO:23, SEQ ID NO:24, SEQ ID NO:25, SEQ ID NO:26, SEQ ID NO:27, SEQ ID NO:28, SEQ ID NO:50, SEQ ID NO:51, or SEQ ID NO:52. In certain embodiments, the polypeptide comprises an amino acid sequence having at least about 95% sequence identity to SEQ ID NO:23 and/or an amino acid sequence having at least about 95% sequence identity to SEQ ID NO:24 or SEQ ID NO:50. In certain embodiments, the polypeptide comprises an amino acid sequence having at least about 95% sequence identity to SEQ ID NO:25 and/or an amino acid sequence having at least about 95% sequence identity to SEQ ID NO:27 or SEQ ID NO:51. In certain embodiments, the polypeptide comprises an amino acid sequence having at least about 95% sequence identity to SEQ ID NO:26 and/or an amino acid sequence having at least about 95% sequence identity to SEQ ID NO:28 or SEQ ID NO:52. In certain embodiments, the polypeptide comprises an amino acid sequence of SEQ ID NO:23 and/or an amino acid sequence of SEQ ID NO:24. In certain embodiments, the polypeptide comprises an amino acid sequence of SEQ ID NO:23 and/or an amino acid sequence of SEQ ID NO:50. In certain embodiments, the polypeptide comprises an amino acid sequence of SEQ ID NO:25 and/or an amino acid sequence of SEQ ID NO:27. In certain embodiments, the polypeptide comprises an amino acid sequence of SEQ ID NO:25 and/or an amino acid sequence of SEQ ID NO:51. In certain embodiments, the polypeptide comprises an amino acid sequence of SEQ ID NO:26 and/or an amino acid sequence of SEQ ID NO:28. In certain embodiments, the polypeptide comprises an amino acid sequence of SEQ ID NO:26 and/or an amino acid sequence of SEQ ID NO:52.

In some embodiments, the RSPO-binding agent is a polypeptide that specifically binds a human RSPO3, wherein the polypeptide comprises an amino acid sequence having at least about 80% sequence identity to SEQ ID NO:38 and/or SEQ ID NO:39. In some embodiments, the polypeptide comprises an amino acid sequence having at least about 80% sequence identity to SEQ ID NO:40 and/or an amino acid sequence having at least about 80% sequence identity to SEQ ID NO:42. In some embodiments, the polypeptide comprises an amino acid sequence having at least about 80% sequence identity to SEQ ID NO:41 and/or an amino acid sequence having at least about 80% sequence identity to SEQ ID NO:43. In certain embodiments, the polypeptide comprises an amino acid sequence having at least about 85%, at least about 90%, at least about 95%, at least about 97%, or at least about 99% sequence identity to SEQ ID NO:38, SEQ ID NO:39, SEQ ID NO:40, SEQ ID NO:41, SEQ ID NO:42, or SEQ ID NO:43. In certain embodiments, the polypeptide comprises an amino acid sequence having at least about 95% sequence identity to SEQ ID NO:38 and/or an amino acid sequence having at least about 95% sequence identity to SEQ ID NO:39. In certain embodiments, the polypeptide comprises an amino acid sequence having at least about 95% sequence identity to SEQ ID NO:40 and/or an amino acid sequence having at least about 95% sequence identity to SEQ ID NO:42. In certain embodiments, the polypeptide comprises an amino acid sequence having at least about 95% sequence identity to SEQ ID NO:41 and/or an amino acid sequence having at least about 95% sequence identity to SEQ ID NO:43. In certain embodiments, the polypeptide comprises an amino acid sequence of SEQ ID NO:38 and/or an amino acid sequence of SEQ ID NO:39. In certain embodiments, the polypeptide comprises an amino acid sequence of SEQ ID NO:40 and/or an amino acid sequence of SEQ ID NO:42. In certain embodiments, the polypeptide comprises an amino acid sequence of SEQ ID NO:41 and/or an amino acid sequence of SEQ ID NO:43.

In certain embodiments, the RSPO-binding agent is a RSPO1-binding agent (e.g., antibody) that competes for specific binding to RSPO1 with an antibody that comprises the CDRs of antibody 89M5. In certain embodiments, the RSPO-binding agent is a RSPO2-binding agent (e.g., antibody) that competes for specific binding to RSPO2 with an antibody that comprises the CDRs of antibody 130M23. In certain embodiments, the RSPO-binding agent is a RSPO3-binding agent (e.g., antibody) that competes for specific binding to RSPO3 with an antibody that comprises the CDRs of antibody 131R010.

In certain embodiments, the RSPO-binding agent is a RSPO1-binding agent (e.g., an antibody) that binds the same epitope, or essentially the same epitope on RSPO1, as an antibody that comprises the CDRs of antibody 89M5. In certain embodiments, the RSPO-binding agent is a RSPO2-binding agent (e.g., an antibody) that binds the same epitope, or essentially the same epitope on RSPO2, as an antibody that comprises the CDRs of antibody 130M23. In certain embodiments, the RSPO-binding agent is a RSPO3-binding agent (e.g., an antibody) that binds the same epitope, or essentially the same epitope on RSPO3, as an antibody that comprises the CDRs of antibody 131R010.

In certain embodiments, the RSPO-binding agent is a RSPO1-binding agent (e.g., an antibody) that binds an epitope on RSPO1 that overlaps with the epitope on RSPO1 bound by an antibody comprising the CDRs of antibody 89M5. In certain embodiments, the RSPO-binding agent is a RSPO2-binding agent (e.g., an antibody) that binds an epitope on RSPO2 that overlaps with the epitope on RSPO2 bound by an antibody comprising the CDRs of antibody 130M23. In certain embodiments, the RSPO-binding agent is a RSPO3-binding agent (e.g., an antibody) that binds an epitope on RSPO3 that overlaps with the epitope on RSPO3 bound by an antibody comprising the CDRs of antibody 131R010.

In certain embodiments, the RSPO-binding agent is a RSPO3-binding agent (e.g., an antibody) disclosed in U.S. Patent Publication No. 20150147333, each of which is hereby incorporated by reference herein in its entirety for all purposes. In certain embodiments, the RSPO-binding agent is anti-RSPO3 antibody 4H1, 4D4, 5C2, 5D6, 5E11, 6E9, 21C2, or 26E11 disclosed in U.S. Patent Publication No. 20150147333. In certain embodiments, the RSPO-binding agent is an anti-RSPO3 antibody comprising the 6 CDRs of anti-RSPO3 antibody 4H1, 4D4, 5C2, 5D6, 5E11, 6E9, 21C2, or 26E11. In certain embodiments, the RSPO-binding agent is an anti-RSPO3 antibody comprising the VH and/or VL region(s) of anti-RSPO3 antibody 4H1, 4D4, 5C2, 5D6, 5E11, 6E9, 21C2, or 26E11. In certain embodiments, the RSPO-binding agent is a RSPO3-binding agent (e.g., an antibody) that binds the same epitope, or essentially the same epitope on RSPO3 as anti-RSPO3 antibody 4H1, 4D4, 5C2, 5D6, 5E11, 6E9, 21C2, or 26E11.

In certain embodiments, the RSPO-binding agent is a RSPO2-binding agent (e.g., an antibody) disclosed in U.S. Patent Publication No. 20150147333, which is hereby incorporated by reference herein in its entirety for all purposes. In certain embodiments, the RSPO-binding agent is anti-RSPO2 antibody 1A1, 11F11, 26E11, 36D2, or 49G5 disclosed in U.S. Patent Publication No. 20150147333. In certain embodiments, the RSPO-binding agent is an anti-RSPO2 antibody comprising the 6 CDRs of anti-RSPO2 antibody 1A1, 11F11, 26E11, 36D2, or 49G5. In certain embodiments, the RSPO-binding agent is an anti-RSPO2 antibody comprising the VH and/or VL region(s) of anti-RSPO2 antibody 1A1, 11F11, 26E11, 36D2, or 49G5. In certain embodiments, the RSPO-binding agent is a RSPO2-binding agent (e.g., an antibody) that binds the same epitope, or essentially the same epitope on RSPO2 as anti-RSPO2 antibody 1A1, 11F11, 26E11, 36D2, or 49G5.

In certain embodiments of the methods described herein, a RSPO-binding agent (e.g., an antibody) binds at least one human RSPO protein and modulates RSPO activity. In some embodiments, the RSPO-binding agent is a RSPO antagonist and decreases RSPO activity. In some embodiments, the RSPO-binding agent is a RSPO antagonist and decreases β-catenin activity.

In certain embodiments, a RSPO1-binding agent (e.g., an antibody) binds human RSPO1 and modulates RSPO1 activity. In some embodiments, a RSPO1-binding agent is a RSPO1 antagonist and decreases RSPO1 activity. In some embodiments, a RSPO1-binding agent is a RSPO1 antagonist and decreases β-catenin activity. In certain embodiments, a RSPO2-binding agent (e.g., an antibody) binds human RSPO2 and modulates RSPO2 activity. In some embodiments, a RSPO2-binding agent is a RSPO2 antagonist and decreases RSPO2 activity. In some embodiments, a RSPO2-binding agent is a RSPO2 antagonist and decreases β-catenin activity. In certain embodiments, a RSPO3-binding agent (e.g., an antibody) binds human RSPO3 and modulates RSPO3 activity. In some embodiments, a RSPO3-binding agent is a RSPO3 antagonist and decreases RSPO3 activity. In some embodiments, a RSPO3-binding agent is a RSPO3 antagonist and decreases β-catenin activity.

In certain embodiments, the RSPO-binding agent (e.g., an antibody) is an antagonist of at least one human RSPO protein. In some embodiments, the RSPO-binding agent is an antagonist of at least one RSPO and inhibits RSPO activity. In certain embodiments, the RSPO-binding agent inhibits RSPO activity by at least about 10%, at least about 20%, at least about 30%, at least about 50%, at least about 75%, at least about 90%, or about 100%. In some embodiments, the RSPO-binding agent inhibits activity of one, two, three, or four RSPO proteins. In some embodiments, the RSPO-binding agent inhibits activity of human RSPO1, RSPO2, RSPO3, and/or RSPO4.

In certain embodiments, the RSPO-binding agent (e.g., antibody) is an antagonist of at least one human RSPO protein. In certain embodiments, the RSPO-binding agent inhibits RSPO signaling by at least about 10%, at least about 20%, at least about 30%, at least about 50%, at least about 75%, at least about 90%, or about 100%. In some embodiments, the RSPO-binding agent inhibits signaling by one, two, three, or four RSPO proteins. In some embodiments, the RSPO-binding agent inhibits signaling of human RSPO1, RSPO2, RSPO3, and/or RSPO4.

In certain embodiments, the RSPO-binding agent (e.g., antibody) is an antagonist of β-catenin signaling. In certain embodiments, the RSPO-binding agent inhibits β-catenin signaling by at least about 10%, at least about 20%, at least about 30%, at least about 50%, at least about 75%, at least about 90%, or about 100%.

In certain embodiments, the RSPO-binding agent (e.g., antibody) inhibits binding of at least one RSPO protein to a receptor. In certain embodiments, the RSPO-binding agent inhibits binding of a human RSPO protein to one or more of its receptors. In some embodiments, the RSPO-binding agent inhibits binding of a RSPO protein to at least one LGR protein. In some embodiments, the RSPO-binding agent inhibits binding of a RSPO protein to LGR4 (SEQ ID NO:53), LGR5 (SEQ ID NO:54), and/or LGR6 (SEQ ID NO:55). In certain embodiments, the inhibition of binding of a RSPO-binding agent to at least one LGR protein is at least about 10%, at least about 25%, at least about 50%, at least about 75%, at least about 90%, or at least about 95%. In certain embodiments, a RSPO-binding agent that inhibits binding of at least one RSPO to at least one LGR protein further inhibits β-catenin signaling.

In certain embodiments, the RSPO-binding agent (e.g., antibody) blocks binding of at least one RSPO to a receptor. In certain embodiments, the RSPO-binding agent blocks binding of a human RSPO protein to one or more of its receptors. In some embodiments, the RSPO-binding agent blocks binding of a RSPO to at least one LGR protein. In some embodiments, the RSPO-binding agent blocks binding of at least one RSPO protein to LGR4 (SEQ ID NO:53), LGR5 (SEQ ID NO:54), and/or LGR6 (SEQ ID NO:55). In certain embodiments, the blocking of binding of a RSPO-binding agent to at least one LGR protein is at least about 10%, at least about 25%, at least about 50%, at least about 75%, at least about 90%, or at least about 95%. In certain embodiments, a RSPO-binding agent that blocks binding of at least one RSPO protein to at least one LGR protein further inhibits β-catenin signaling.

In certain embodiments, the RSPO-binding agent (e.g., an antibody) inhibits β-catenin signaling. It is understood that a RSPO-binding agent that inhibits β-catenin signaling can, in certain embodiments, inhibit signaling by one or more receptors in the β-catenin signaling pathway but not necessarily inhibit signaling by all receptors. In certain alternative embodiments, β-catenin signaling by all human receptors can be inhibited. In certain embodiments, β-catenin signaling by one or more receptors selected from the group consisting of LGR4 (SEQ ID NO:53), LGR5 (SEQ ID NO:54), and/or LGR6 (SEQ ID NO:55) is inhibited. In certain embodiments, the inhibition of β-catenin signaling by a RSPO-binding agent is a reduction in the level of β-catenin signaling of at least about 10%, at least about 25%, at least about 50%, at least about 75%, at least about 90%, or at least about 95%.

In certain embodiments, the RSPO-binding agent (e.g., an antibody) inhibits activation of β-catenin. It is understood that a RSPO-binding agent that inhibits activation of β-catenin can, in certain embodiments, inhibit activation of β-catenin by one or more receptors, but not necessarily inhibit activation of β-catenin by all receptors. In certain alternative embodiments, activation of β-catenin by all human receptors can be inhibited. In certain embodiments, activation of β-catenin by one or more receptors selected from the group consisting of LGR4 (SEQ ID NO:53), LGR5 (SEQ ID NO:54), and LGR6 (SEQ ID NO:55) is inhibited. In certain embodiments, the inhibition of activation of β-catenin by a RSPO-binding agent is a reduction in the level of activation of β-catenin of at least about 10%, at least about 25%, at least about 50%, at least about 75%, at least about 90%, or at least about 95%.

In certain embodiments, the RSPO-LGR pathway inhibitors are agents that bind one or more human LGR proteins. These agents are referred to herein as “LGR-binding agents”. Non-limiting examples of LGR-binding agents can be found in U.S. Pat. Nos. 8,158,758, 8,158,757, 8,802,097, and U.S. Patent Publication Nos. 2012/0135422, 2013/0209473, 2014/0044713, each of which is hereby incorporated by reference herein in its entirety for all purposes.

In some embodiments, the LGR-binding agent binds at least one human LGR protein. In alternative embodiments, the LGR-binding agent binds two or more human LGR proteins. In some embodiments, the LGR-binding agent is an antibody. In some embodiments, the LGR-binding agent inhibits (partially or wholly) the binding of at least one RSPO protein (e.g., RSPO1, RSPO2, RSPO3, and/or RSPO4) to at least one LGR protein (e.g., LGR4, LGR5, and/or LGR6). In certain embodiments, the LGR-binding agent inhibits RSPO-activated LGR signaling, such as LGR5 signaling. In certain embodiments, the LGR-binding agent inhibits β-catenin signaling.

In certain embodiments, a LGR-binding agent is an antibody, for example, an antibody that binds at least one LGR protein. Thus, the LGR-binding agent can be an antibody that specifically binds LGR5. In certain alternative embodiments, the LGR-binding agent is an antibody that specifically binds LGR4 or LGR6.

In certain embodiments, a LGR-binding agent is an antibody that specifically binds at least one human LGR protein. In certain embodiments, the antibody specifically binds at least one human LGR protein selected from the group consisting of LGR4, LGR5, and LGR6. In certain embodiments, the antibody specifically binds LGR5. In certain embodiments, the antibody specifically binds two or more human LGR proteins selected from the group consisting of LGR4, LGR5, and LGR6. In certain embodiments, the antibody that specifically binds at least one human LGR protein, also inhibits binding of at least one RSPO protein (e.g., RSPO1, RSPO2, RSPO3, and/or RSPO4) to the at least one human LGR protein (e.g., LGR5). In certain embodiments, the antibody that specifically binds at least one human LGR protein is characterized by an ability to inhibit RSPO activation of LGR signaling and/or an ability to inhibit β-catenin signaling. In certain embodiments, the antibody that specifically binds at least one human LGR protein is characterized by the ability to inhibit tumor growth, such as the growth of a solid tumor. In certain embodiments, the antibody that specifically binds at least one human LGR protein is characterized by the ability to inhibit tumor growth, such as the growth of a solid tumor comprising solid tumor stem cells. For example, in some embodiments, the antibody that specifically binds at least one human LGR protein, disrupts or inhibits RSPO binding to LGR, and inhibits tumor growth. In certain alternative embodiments, the antibody that specifically binds at least one LGR protein, also disrupts RSPO activation of LGR signaling and inhibits tumor growth. In certain alternative embodiments, the antibody that specifically binds at least one LGR protein, also inhibits RSPO activation of LGR signaling and/or β-catenin signaling and inhibits tumor growth.

In certain embodiments, a LGR-binding agent that inhibits binding of a RSPO protein to a LGR protein, inhibits at least about 25%, at least about 50%, at least about 60%, at least about 70%, at least about 80%, or at least about 90% of the binding of the RSPO protein to a LGR protein in an in vitro or in vivo assay.

Likewise, in certain embodiments, a LGR-binding agent that inhibits (a) RSPO activation of LGR signaling and/or (b) β-catenin signaling, inhibits at least about 25%, at least about 50%, at least about 60%, at least about 70%, at least about 80%, or at least about 90% of the signaling in an in vitro or in vivo assay.

In certain embodiments, a LGR-binding agent is an isolated antibody that specifically binds to an extracellular domain of a human LGR protein and inhibits growth of a solid tumor. In certain embodiments, a LGR-binding agent is an isolated antibody that specifically binds to an extracellular domain of a human LGR protein and inhibits growth of a solid tumor comprising solid tumor stem cells. In certain embodiments, the extracellular domain comprises amino acids 22-564 of human LGR5 (SEQ ID NO:56). In certain embodiments, the antibody is a monoclonal antibody. In certain embodiments, the antibody is a humanized or human antibody.

In certain embodiments, a LGR-binding agent is an isolated antibody that specifically binds to an extracellular domain of a human LGR protein and disrupts RSPO activation of LGR signaling. In certain embodiments, the extracellular domain comprises amino acids 22-564 of human LGR5 (SEQ ID NO:56). In certain embodiments, the antibody is a monoclonal antibody. In certain embodiments, the antibody is a humanized or human antibody.

In certain embodiments, a LGR-binding agent is monoclonal anti-LGR5 antibody 88M1. The 88M1 monoclonal antibody is produced by a hybridoma cell line deposited with the American Type Culture collection (ATCC), 10801 University Blvd, Manassas, Va., 20110, USA, on Jul. 2, 2008, in accordance with the Budapest Treaty, under ATCC deposit number PTA-9342. In certain embodiments, a LGR-binding agent is an antibody that specifically binds human LGR5 and (a) comprises a heavy chain variable region that has at least about 95% sequence identity (e.g., at least about 98% or about 100% sequence identity) to the heavy chain variable region of 88M1; (b) comprises a light chain variable region that has at least about 95% (e.g., at least about 98% or about 100% sequence identity) sequence identity to the light chain variable region of 88M1; (c) comprises the heavy chain CDRs of 88M1; (d) comprises the light chain CDRs of 88M1; (e) binds to an epitope that 88M1 binds to; and/or (f) competes with 88M1 in a competitive binding assay.

In certain embodiments, the RSPO-binding agent or LGR-binding agent is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgA, IgD, IgE, IgG, or IgM antibody. In certain embodiments, the antibody is an IgG1 antibody. In certain embodiments, the antibody is an IgG2 antibody. In some embodiments, the antibody is an IgG4 antibody. In certain embodiments, the antibody is an antibody fragment comprising an antigen-binding site. In some embodiments, the antibody is a bispecific antibody or a multispecific antibody. In some embodiments, the antibody is a monovalent antibody. In some embodiments, the antibody is a monospecific antibody. In some embodiments, the antibody is a bivalent antibody. In some embodiments, the antibody is conjugated to a cytotoxic moiety. In some embodiments, the antibody is isolated. In some embodiments, the antibody is substantially pure.

RSPO-binding agents and LGR-binding agents (e.g., antibodies) can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blot analysis, radioimmunoassay, ELISA, “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, and protein A immunoassay. Such assays are routine and well-known in the art (see, e.g., Ausubel et al., Editors, 1994-present, Current Protocols in Molecular Biology, John Wiley & Sons, Inc., New York, N.Y.).

For example, the specific binding of an agent (e.g., RSPO-binding agent or LGR-binding agent) to a human RSPO protein or human LGR protein can be determined using ELISA. An ELISA assay comprises preparing antigen, coating wells of a 96 well microtiter plate with antigen, adding the RSPO-binding agent or LGR-binding agent conjugated to a detectable compound such as an enzymatic substrate (e.g. horseradish peroxidase or alkaline phosphatase) to the well, incubating for a period of time, and detecting the presence of the agent bound to the antigen. In some embodiments, the RSPO-binding agent or LGR-binding agent is not conjugated to a detectable compound, but instead a second antibody that recognizes the RSPO-binding agent or LGR-binding agent (e.g., an anti-Fc antibody) and is conjugated to a detectable compound is added to the well. In some embodiments, instead of coating the well with the antigen, the RSPO-binding agent or LGR-binding agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs known in the art.

In another example, the specific binding of an agent (e.g., RSPO-binding agent or LGR-binding agent) to a human RSPO protein or human LGR protein can be determined using FACS. A FACS screening assay can comprise generating a cDNA construct that expresses an antigen (e.g., RSPO or LGR), optionally as a fusion protein (e.g., RSPO-CD4TM or LGR-CD4TM), transfecting the construct into cells, expressing the antigen on the surface of the cells, mixing the RSPO-binding agent or LGR-binding agent with the transfected cells, and incubating for a period of time. The cells bound by the RSPO-binding agent or LGR-binding agent can be identified using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that can enhance screening (e.g., screening for blocking antibodies).

The binding affinity of an agent (e.g., RSPO-binding agent or LGR-binding agent) to an antigen and the off-rate of an agent-antigen interaction can be determined by competitive binding assays. One example of a competitive binding assay is a radioimmunoassay comprising the incubation of labeled antigen (e.g., labeled with ³H or ¹²⁵I), or fragment or variant thereof, with a binding agent of interest in the presence of increasing amounts of unlabeled antigen followed by the detection of the agent bound to the labeled antigen. The affinity of the agent for the antigen and the binding off-rates can be determined from the data by Scatchard plot analysis. In some embodiments, Biacore kinetic analysis is used to determine the binding on and off rates of agents that bind an antigen. In some embodiments, Biacore kinetic analysis comprises analyzing the binding and dissociation of antibodies from chips with immobilized antigen on their surface. In some embodiments, Biacore kinetic analysis comprises analyzing the binding and dissociation of antigen from chips with immobilized binding agent on their surface.

In vivo and in vitro assays for determining whether a RSPO-binding agent or LGR-binding agent inhibits β-catenin signaling are known in the art. For example, cell-based, luciferase reporter assays utilizing a TCF/Luc reporter vector containing multiple copies of the TCF-binding domain upstream of a firefly luciferase reporter gene can be used to measure β-catenin signaling levels in vitro (Gazit et al., 1999, Oncogene, 18; 5959-66; TOPflash, Millipore, Billerica Mass.). The level of β-catenin signaling in the presence of one or more Wnts (e.g., Wnt(s) expressed by transfected cells or provided by Wnt-conditioned media) with or without a RSPO protein or RSPO-conditioned media in the presence of a RSPO-binding agent or LGR-binding agent is compared to the level of signaling without the RSPO-binding agent or LGR-binding agent present. In addition to the TCF/Luc reporter assay, the effect of a RSPO-binding agent or LGR-binding agent on β-catenin signaling can be measured in vitro or in vivo by measuring the effect of the agent on the level of expression of β-catenin-regulated genes, such as c-myc (He et al., 1998, Science, 281:1509-12), cyclin D1 (Tetsu et al., 1999, Nature, 398:422-6) and/or fibronectin (Gradl et al. 1999, Mol. Cell Biol., 19:5576-87). In certain embodiments, the effect of a RSPO-binding agent or LGR-binding agent on β-catenin signaling can also be assessed by measuring the effect of the agent on the phosphorylation state of Dishevelled-1, Dishevelled-2, Dishevelled-3, LRP5, LRP6, and/or β-catenin.

In some embodiments, the RSPO-LGR pathway inhibitors (e.g., RSPO-binding agent and LGR-binding agent) are polyclonal antibodies. Polyclonal antibodies can be prepared by any known method. In some embodiments, polyclonal antibodies are generated by immunizing an animal (e.g., a rabbit, rat, mouse, goat, donkey) by multiple subcutaneous or intraperitoneal injections of the relevant antigen (e.g., a purified peptide fragment, full-length recombinant protein, or fusion protein). The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood and/or ascites of the immunized animal. The polyclonal antibodies can be purified from serum or ascites according to standard methods in the art including, but not limited to, affinity chromatography, ion-exchange chromatography, gel electrophoresis, and dialysis.

In some embodiments, the RSPO-LGR pathway inhibitors (e.g., RSPO-binding agent or LGR-binding agent) are monoclonal antibodies. Monoclonal antibodies can be prepared using hybridoma methods known to one of skill in the art. In some embodiments, using the hybridoma method, a mouse, hamster, or other appropriate host animal, is immunized as described above to elicit from lymphocytes the production of antibodies that will specifically bind the immunizing antigen. In some embodiments, lymphocytes can be immunized in vitro. In some embodiments, the immunizing antigen can be a human protein or a portion thereof. In some embodiments, the immunizing antigen can be a mouse protein or a portion thereof.

Following immunization, lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen can be identified by a variety of methods including, but not limited to, immunoprecipitation, immunoblotting, and in vitro binding assay (e.g., flow cytometry, FACS, ELISA, and radioimmunoassay). The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques known to one skilled in the art. The polynucleotides encoding a monoclonal antibody are isolated from mature B-cells or hybridoma cells, such as by RT-PCR using oligonucleotide primers that specifically amplify genes encoding the heavy and light chains of the antibody, and their sequence is determined using conventional techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins. In other embodiments, recombinant monoclonal antibodies, or fragments thereof, can be isolated from phage display libraries.

The polynucleotide(s) encoding a monoclonal antibody can be further modified in a number of different manners using recombinant DNA technology to generate alternative antibodies. In some embodiments, the constant domains of the light and heavy chains of, for example, a mouse monoclonal antibody can be substituted for those regions of, for example, a human antibody to generate a chimeric antibody, or for a non-immunoglobulin polypeptide to generate a fusion antibody. In some embodiments, the constant regions are truncated or removed to generate the desired antibody fragment of a monoclonal antibody. In some embodiments, site-directed or high-density mutagenesis of the variable region can be used to optimize specificity, affinity, etc. of a monoclonal antibody.

In some embodiments, the RSPO-LGR pathway inhibitor (e.g., RSPO-binding agent or LGR-binding agent) is a humanized antibody. Typically, humanized antibodies are human immunoglobulins in which residues from the CDRs are replaced by residues from CDRs of a non-human species (e.g., mouse, rat, rabbit, hamster, etc.) that have the desired specificity, affinity, and/or binding capability using methods known to one skilled in the art. In some embodiments, the framework region residues of a human immunoglobulin are replaced with the corresponding residues in an antibody from a non-human species. In some embodiments, the humanized antibody can be further modified by the substitution of additional residues either in the framework region and/or within the replaced non-human residues to refine and optimize antibody specificity, affinity, and/or capability. In general, the humanized antibody will comprise variable domain regions containing all, or substantially all, of the CDRs that correspond to the non-human immunoglobulin whereas all, or substantially all, of the framework regions are those of a human immunoglobulin sequence. In some embodiments, the humanized antibody can also comprise at least a portion of an immunoglobulin constant region or domain (Fc), typically that of a human immunoglobulin. In certain embodiments, such humanized antibodies are used therapeutically because they can reduce antigenicity and HAMA (human anti-mouse antibody) responses when administered to a human subject.

In certain embodiments, the RSPO-LGR pathway inhibitor (e.g., RSPO-binding agent or LGR-binding agent) is a human antibody. Human antibodies can be directly prepared using various techniques known in the art. In some embodiments, immortalized human B lymphocytes immunized in vitro or isolated from an immunized individual that produces an antibody directed against a target antigen can be generated. In some embodiments, the human antibody can be selected from a phage library, where that phage library expresses human antibodies. Alternatively, phage display technology can be used to produce human antibodies and antibody fragments in vitro, from immunoglobulin variable domain gene repertoires from unimmunized donors. Techniques for the generation and use of antibody phage libraries are well-known in the art and antibody phage libraries are commercially available. Affinity maturation strategies including, but not limited to, chain shuffling and site-directed mutagenesis, are known in the art and can be employed to generate high affinity human antibodies.

In some embodiments, human antibodies can be made in transgenic mice that contain human immunoglobulin loci. These mice are capable, upon immunization, of producing the full repertoire of human antibodies in the absence of endogenous immunoglobulin production.

In certain embodiments, the RSPO-LGR pathway inhibitor (e.g., RSPO-binding agent or LGR-binding agent) is a bispecific antibody that specifically recognizes at least one human RSPO protein or at least one LGR protein. Bispecific antibodies are capable of specifically recognizing and binding at least two different epitopes. The different epitopes can either be within the same molecule (e.g., two different epitopes on human RSPO3) or on different molecules (e.g., one epitope on RSPO3 and a different epitope on a second protein). In some embodiments, the bispecific antibodies are monoclonal human or humanized antibodies. In some embodiments, the bispecific antibodies are intact antibodies. In some embodiments, the bispecific antibodies are antibody fragments. In certain embodiments, the antibodies are multispecific. In some embodiments, the antibodies can specifically recognize and bind a first antigen target, (e.g., a LGR protein) as well as a second antigen target, such as an effector molecule on a leukocyte (e.g., CD2, CD3, CD28, CD80, or CD86) or a Fc receptor (e.g., CD64, CD32, or CD16) so as to focus cellular defense mechanisms to the cell expressing the first antigen target. In some embodiments, the antibodies can be used to direct cytotoxic agents to cells which express a particular target antigen. These antibodies possess an antigen-binding arm and an arm which binds a cytotoxic agent or a radionuclide chelator, such as EOTUBE, DPTA, DOTA, or TETA. Techniques for making bispecific or multispecific antibodies are known by those skilled in the art.

In certain embodiments, the RSPO-LGR pathway inhibitor (e.g., RSPO-binding agent or LGR-binding agent) is a monospecific antibody. For example, in certain embodiments, each of the one or more antigen-binding sites that an antibody contains is capable of binding (or binds) a homologous epitope on different proteins.

In certain embodiments, the RSPO-LGR pathway inhibitor is an antibody fragment comprising an antigen-binding site. Antibody fragments can have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries to allow rapid and effective identification of monoclonal Fab fragments with the desired specificity for a RSPO or LGR protein or derivatives, fragments, analogs or homologs thereof. In some embodiments, antibody fragments are linear antibody fragments. In certain embodiments, antibody fragments are monospecific or bispecific. In certain embodiments, the RSPO-LGR pathway inhibitor is a scFv. Various techniques can be used for the production of single-chain antibodies specific to one or more human RSPO proteins or one or more human LGR proteins.

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis). In some embodiments, an antibody is modified to decrease its serum half-life.

In certain embodiments, the RSPO-LGR pathway inhibitor is a heteroconjugate antibody. Heteroconjugate antibodies are composed of two covalently joined antibodies. Such antibodies have, for example, been proposed to target immune cells to unwanted cells. It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

It should be appreciated that modified antibodies can comprise any type of variable region that provides for the association of the antibody with the target (i.e., a human RSPO protein or a human LGR protein). In this regard, the variable region can comprise or be derived from any type of mammal that can be induced to mount a humoral response and generate immunoglobulins against the desired tumor-associated antigen. As such, the variable region of the modified antibodies can be, for example, of human, murine, non-human primate (e.g. cynomolgus monkeys, macaques, etc.) or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification and/or alteration. Although the CDRs can be derived from an antibody of the same class or even subclass as the antibody from which the framework regions are derived, it is envisaged that the CDRs will be derived preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the activity of the antigen-binding site.

Alterations to the variable region notwithstanding, those skilled in the art will appreciate that the modified antibodies will comprise antibodies (e.g., full-length antibodies or immunoreactive fragments thereof) in which at least a fraction of one or more of the constant region domains has been deleted or otherwise altered so as to provide desired biochemical characteristics such as increased tumor localization and/or increased serum half-life when compared with an antibody of approximately the same immunogenicity comprising a native or unaltered constant region. In some embodiments, the constant region of the modified antibodies will comprise a human constant region. Modifications to the constant region comprise additions, deletions or substitutions of one or more amino acids in one or more domains. The modified antibodies disclosed herein can comprise alterations or modifications to one or more of the three heavy chain constant domains (CH1, CH2, or CH3) and/or to the light chain constant domain (CL). In some embodiments, one or more domains are partially or entirely deleted from the constant regions of the modified antibodies. In some embodiments, the modified antibodies will comprise domain deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs can be expressed wherein the CH2 domain has been deleted and the remaining CH3 domain (modified or unmodified) is joined to the hinge region with a 5-20 amino acid spacer. Such a spacer can be added to ensure that the regulatory elements of the constant domain remain free and accessible or that the hinge region remains flexible. However, it should be noted that amino acid spacers can, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies can have only a partial deletion of a constant domain or substitution of a few or even a single amino acid. For example, the mutation of a single amino acid in selected areas of the CH2 domain can be enough to substantially reduce Fc binding and thereby increase cancer cell localization and/or tumor penetration. Similarly, it can be desirable to simply delete the part of one or more constant region domains that control a specific effector function (e.g. complement C1q binding). Such partial deletions of the constant regions can improve selected characteristics of the antibody (serum half-life) while leaving other desirable functions associated with the subject constant region domain intact. Moreover, as alluded to above, the constant regions of the disclosed antibodies can be modified through the mutation or substitution of one or more amino acids that enhances the profile of the resulting construct. In this respect it can be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells, release of inflammatory mediators, placental transfer, and control of immunoglobulin production.

In certain embodiments, the RSPO-LGR pathway inhibitors are antibodies that provide for altered effector functions. These altered effector functions can affect the biological profile of the administered antibody. For example, in some embodiments, the deletion or inactivation (through point mutations or other means) of a constant region domain can reduce Fc receptor binding of the circulating modified antibody (e.g., anti-RSPO antibody) thereby increasing cancer cell localization and/or tumor penetration. In other embodiments, the constant region modifications increase or reduce the serum half-life of the antibody. In some embodiments, the constant region is modified to eliminate disulfide linkages or oligosaccharide moieties. Modifications to the constant region can easily be made using well known biochemical or molecular engineering techniques well within the purview of the skilled artisan.

In certain embodiments, a RSPO-LGR pathway inhibitor is an antibody that does not have one or more effector functions. For instance, in some embodiments, the antibody has no ADCC activity, and/or no CDC activity. In certain embodiments, the antibody does not bind an Fc receptor, and/or complement factors. In certain embodiments, the antibody has no effector function.

Variants and equivalents which are substantially homologous to the chimeric, humanized, and human antibodies, or antibody fragments thereof, set forth herein can also be used in the methods described herein. These can contain, for example, conservative substitution mutations.

In certain embodiments, the antibodies described herein are isolated. In certain embodiments, the antibodies described herein are substantially pure.

In some embodiments, the RSPO-LGR pathway inhibitor is a soluble receptor. In certain embodiments, the RSPO-binding agent is a soluble receptor. In certain embodiments, the soluble receptor comprises the extracellular domain of a LGR protein or fragment of the extracellular domain of a LGR protein. In certain embodiments, the LGR protein is LGR5. For example, in some embodiments, the RSPO-binding agent is a fusion protein comprising a fragment of the LGR5 receptor. In some embodiments, the RSPO-binding agent is a fusion protein comprising a fragment of the LGR5 receptor and the Fc portion of an antibody.

In certain embodiments, the RSPO-binding agent is a soluble receptor comprising an extracellular domain of a human LGR protein or a fragment thereof that inhibits growth of a solid tumor comprising solid tumor stem cells. In certain embodiments, the extracellular domain comprises amino acids 22-564 of human LGR5 (SEQ ID NO:56). In certain embodiments, the extracellular domain of human LGR5 is linked in-frame to a non-LGR protein sequence. In certain embodiments, the non-LGR protein is human Fc.

In certain embodiments, the RSPO-binding agent is a soluble receptor comprising an extracellular domain of a human LGR protein or a fragment thereof that inhibits RSPO activation of LGR signaling. In certain embodiments, the extracellular domain comprises amino acids 22-564 of human LGR5 (SEQ ID NO:56). In certain embodiments, the extracellular domain of human LGR5 is linked in-frame to a non-LGR protein sequence. In certain embodiments, the non-LGR protein is human Fc. Non-limiting examples of soluble LGR receptors can be found in U.S. Pat. Nos. 8,158,758 and 8,158,757, each of which is hereby incorporated by reference herein in its entirety for all purposes.

In certain embodiments, the RSPO-binding agent is a soluble receptor comprising an extracellular domain of a human LGR protein that inhibits growth of a solid tumor. In certain embodiments, the RSPO-binding agent is a soluble receptor comprising an extracellular domain of a human LGR protein that inhibits growth of a solid tumor comprising solid tumor stem cells. In certain embodiments, the extracellular domain comprises amino acids 22-564 of human LGR5 (SEQ ID NO:56). In certain embodiments, the extracellular domain comprises a fragment of the amino acids 22-564 of human LGR5 (SEQ ID NO:56). In certain embodiments, the extracellular domain of human LGR5 or fragment thereof is linked in-frame to a non-LGR protein sequence. In certain embodiments, the non-LGR protein is human Fc.

In certain embodiments, the soluble receptor comprises a variant of the aforementioned extracellular domain of a human LGR protein that comprises one or more (e.g., one, two, three, four, five, six, seven, eight, nine, ten, etc.) conservative substitutions and is capable of binding RSPO protein(s).

In certain embodiments, the soluble receptor, such as an agent comprising an extracellular domain of a human LGR protein, further comprises a non-LGR (e.g., heterologous) polypeptide. In some embodiments, a soluble receptor can include a LGR ECD linked to other non-LGR functional and structural polypeptides including, but not limited to, a human Fc region, at least one protein tag (e.g., myc, FLAG, GST, GFP), other endogenous proteins or protein fragments, or any other useful protein sequence including any linker region between a LGR ECD and a second polypeptide. In certain embodiments, the non-LGR polypeptide comprises a human Fc region. The Fc region can be obtained from any of the classes of immunoglobulin, IgG, IgA, IgM, IgD and IgE. In some embodiments, the Fc region is a human IgG1 Fc region. In some embodiments, the Fc region is a human IgG2 Fc region. In some embodiments, the Fc region is a wild-type Fc region. In some embodiments, the Fc region is a mutated Fc region. In some embodiments, the Fc region is truncated at the N-terminal end by 1, 2, 3, 4, 5, 6, 7, 8, 9, or 10 amino acids, (e.g., in the hinge domain). In some embodiments, an amino acid in the hinge domain is changed to hinder undesirable disulfide bond formation. In some embodiments, a cysteine is replaced with a serine to hinder undesirable disulfide bond formation. In some embodiments, the Fc region is truncated at the C-terminal end by 1, 2, 3, or more amino acids. In some embodiments, the Fc region is truncated at the C-terminal end by 1 amino acid. In certain embodiments, the non-LGR polypeptide comprises SEQ ID NO:58, SEQ ID NO:59, SEQ ID NO:60, SEQ ID NO:61, or SEQ ID NO:62. In certain embodiments, the non-LGR polypeptide consists essentially of SEQ ID NO:58, SEQ ID NO:59, SEQ ID NO:60, SEQ ID NO:61, or SEQ ID NO:62. In certain embodiments, the non-LGR polypeptide comprises SEQ ID NO:61. In certain embodiments, the non-LGR polypeptide consists essentially of SEQ ID NO:61.

In certain embodiments, a soluble receptor is a fusion protein comprising an extracellular domain of a LGR polypeptide capable of binding a RSPO protein and a Fc region. As used herein, a “fusion protein” is a hybrid protein expressed by a nucleic acid molecule comprising nucleotide sequences of at least two genes. In some embodiments, the C-terminus of the first polypeptide is linked to the N-terminus of the immunoglobulin Fc region. In some embodiments, the first polypeptide (e.g., an extracellular domain of a LGR polypeptide) is directly linked to the Fc region (i.e. without an intervening peptide linker). In some embodiments, the first polypeptide is linked to the Fc region via a linker.

In some embodiments, the fusion protein comprises SEQ ID NO:57. In some embodiments, the fusion protein comprises SEQ ID NO:63. In some embodiments, the soluble receptor comprises SEQ ID NO:63. In some embodiments, the RSPO-binding agent comprises SEQ ID NO:63.

As used herein, the term “linker” refers to a linker inserted between a first polypeptide (e.g., a LGR component) and a second polypeptide (e.g., a Fc region). In some embodiments, the linker is a peptide linker. Linkers should not adversely affect the expression, secretion, or bioactivity of the polypeptide. Linkers should not be antigenic and should not elicit an immune response. Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. As used herein, a “linker” is an intervening peptide sequence that does not include amino acid residues from either the C-terminus of the first polypeptide (e.g., LGR component) or the N-terminus of the second polypeptide (e.g., a Fc region).

In certain embodiments, a RSPO-binding agent (e.g., soluble receptor) comprises a Fc region of an immunoglobulin. Those skilled in the art will appreciate that some of the binding agents will comprise fusion proteins in which at least a portion of the Fc region has been deleted or otherwise altered so as to provide desired biochemical characteristics, such as increased cancer cell localization, increased tumor penetration, reduced serum half-life, or increased serum half-life, when compared with a fusion protein of approximately the same immunogenicity comprising a native or unaltered constant region. Modifications to the Fc region can include additions, deletions, or substitutions of one or more amino acids in one or more domains. The modified fusion proteins disclosed herein can comprise alterations or modifications to one or more of the two heavy chain constant domains (CH2 or CH3) or to the hinge region. In other embodiments, the entire CH2 domain can be removed (ΔCH2 constructs). In some embodiments, the omitted constant region domain is replaced by a short amino acid spacer (e.g., 10 residues) that provides some of the molecular flexibility typically imparted by the absent constant region domain.

In some embodiments, the modified fusion proteins are engineered to link the CH3 domain directly to the hinge region. In other embodiments, a peptide spacer is inserted between the hinge region and the modified CH2 and/or CH3 domains. For example, constructs can be expressed wherein the CH2 domain has been deleted and the remaining CH3 domain (modified or unmodified) is joined to the hinge region with a 5-20 amino acid spacer. Such a spacer can be added to ensure that the regulatory elements of the constant domain remain free and accessible or that the hinge region remains flexible. However, it should be noted that amino acid spacers can, in some cases, prove to be immunogenic and elicit an unwanted immune response against the construct. Accordingly, in certain embodiments, any spacer added to the construct will be relatively non-immunogenic so as to maintain the desired biological qualities of the fusion protein.

In some embodiments, the modified fusion proteins can have only a partial deletion of a constant domain or substitution of a few or even a single amino acid. For example, the mutation of a single amino acid in selected areas of the CH2 domain can be enough to substantially reduce Fc binding and thereby increase cancer cell localization and/or tumor penetration. Similarly, it can be desirable to simply delete that part of one or more constant region domains that control a specific effector function (e.g., complement C1q binding). Such partial deletions of the constant regions can improve selected characteristics of the binding agent (e.g., serum half-life) while leaving other desirable functions associated with the subject constant region domain intact. Moreover, as alluded to above, the constant regions of the disclosed fusion proteins can be modified through the mutation or substitution of one or more amino acids that enhances the profile of the resulting construct. In this respect it can be possible to disrupt the activity provided by a conserved binding site (e.g., Fc binding) while substantially maintaining the configuration and immunogenic profile of the modified fusion protein. In certain embodiments, the modified fusion proteins comprise the addition of one or more amino acids to the constant region to enhance desirable characteristics such as decreasing or increasing effector function, or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an immunoglobulin can bind to a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells, release of inflammatory mediators, placental transfer, and control of immunoglobulin production.

In some embodiments, the modified fusion proteins provide for altered effector functions that, in turn, affect the biological profile of the administered agent. For example, in some embodiments, the deletion or inactivation (through point mutations or other means) of a constant region domain can reduce Fc receptor binding of the circulating modified agent, thereby increasing cancer cell localization and/or tumor penetration. In other embodiments, the constant region modifications increase or reduce the serum half-life of the agent. In some embodiments, the constant region is modified to eliminate disulfide linkages or oligosaccharide moieties.

In certain embodiments, a modified fusion protein does not have one or more effector functions normally associated with an Fc region. In some embodiments, the agent has no antibody-dependent cell-mediated cytotoxicity (ADCC) activity, and/or no complement-dependent cytotoxicity (CDC) activity. In certain embodiments, the agent does not bind to the Fc receptor and/or complement factors. In certain embodiments, the agent has no effector function.

In some embodiments, the RSPO-binding agent (e.g., a soluble receptor) described herein is modified to reduce immunogenicity. In general, immune responses against completely normal human proteins are rare when these proteins are used as therapeutics. However, although many fusion proteins comprise polypeptides sequences that are the same as the sequences found in nature, several therapeutic fusion proteins have been shown to be immunogenic in mammals. In some studies, a fusion protein comprising a linker has been found to be more immunogenic than a fusion protein that does not contain a linker. Accordingly, in some embodiments, the polypeptides are analyzed by computation methods to predict immunogenicity. In some embodiments, the polypeptides are analyzed for the presence of T-cell and/or B-cell epitopes. If any T-cell or B-cell epitopes are identified and/or predicted, modifications to these regions (e.g., amino acid substitutions) can be made to disrupt or destroy the epitopes. Various algorithms and software that can be used to predict T-cell and/or B-cell epitopes are known in the art. For example, the software programs SYFPEITHI, HLA Bind, PEPVAC, RANKPEP, DiscoTope, ElliPro, and Antibody Epitope Prediction are all publicly available.

In some embodiments, the RSPO-LGR pathway inhibitors are polypeptides. The polypeptides can be recombinant polypeptides, natural polypeptides, or synthetic polypeptides comprising an antibody, or fragment thereof, that bind at least one human RSPO protein or at least one LGR protein. It will be recognized in the art that some amino acid sequences can be varied without significant effect on the structure or function of the protein. Thus, the methods described herein further encompass using variations of the polypeptides which show substantial activity or which include regions of an antibody, or fragment thereof, against a human RSPO protein or a LGR protein. In some embodiments, amino acid sequence variations of RSPO-binding polypeptides or LGR-binding polypeptides can include deletions, insertions, inversions, repeats, and/or other types of substitutions.

The polypeptides, analogs and variants thereof, can be further modified to contain additional chemical moieties not normally part of the polypeptide. The derivatized moieties can improve the solubility, the biological half-life, and/or absorption of the polypeptide. The moieties can also reduce or eliminate any undesirable side effects of the polypeptides and variants. An overview for chemical moieties can be found in Remington: The Science and Practice of Pharmacy, 22^{nd} Edition, 2012, Pharmaceutical Press, London.

Many proteins, including antibodies and soluble receptors, contain a signal sequence that directs the transport of the proteins to various locations. Signal sequences (also referred to as signal peptides or leader sequences) are located at the N-terminus of nascent polypeptides (e g, amino acids 1-21 of human LGR5 (SEQ ID NO:54)). They target the polypeptide to the endoplasmic reticulum and the proteins are sorted to their destinations, for example, to the inner space of an organelle, to an interior membrane, to the cell's outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site can be recognized and/or can be used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides as described herein can comprise a mixture of polypeptides with different N-termini. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc) amino acid substitutions and/or deletions as compared to a “native” or “parental” signal sequence. In some embodiments, the signal sequence of the polypeptide comprises amino acid substitutions and/or deletions that allow one cleavage site to be dominant, thereby resulting in a substantially homogeneous polypeptide with one N-terminus. In some embodiments, a signal sequence of the polypeptide affects the expression level of the polypeptide, e.g., increased expression or decreased expression.

The isolated polypeptides described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof.

In some embodiments, a DNA sequence encoding a polypeptide of interest can be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

Recombinant expression vectors can be used to amplify and express DNA encoding agents (e.g., antibodies or soluble receptors), or fragments thereof, which bind a human RSPO protein or a human LGR protein. For example, recombinant expression vectors can be replicable DNA constructs which have synthetic or cDNA-derived DNA fragments encoding a polypeptide chain of a RSPO-binding agent, a LGR-binding agent, an anti-RSPO antibody or fragment thereof, an anti-LGR antibody or fragment thereof, or a LGR-Fc soluble receptor operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems can include a leader sequence enabling extracellular secretion of translated protein by a host yeast cell. Where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of a RSPO-binding or LGR-binding agent (or a protein to use as an antigen) include prokaryotes, yeast cells, insect cells, or higher eukaryotic cells. Prokaryotes include gram-negative or gram-positive organisms, for example E. coli or Bacillus. Higher eukaryotic cells include established cell lines of mammalian origin as described below. Cell-free translation systems can also be employed. Appropriate cloning and expression vectors for use with bacterial, fungal, yeast, and mammalian cellular hosts are known to those skilled in the art.

Various mammalian cell culture systems are used to express recombinant polypeptides. Expression of recombinant proteins in mammalian cells can be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

The proteins produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Isolated proteins can also be physically characterized using such techniques as proteolysis, mass spectrometry (MS), nuclear magnetic resonance (NMR), high performance liquid chromatography (HPLC), and x-ray crystallography.

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a binding agent. Some or all of the foregoing purification steps, in various combinations, can also be employed to provide a homogeneous recombinant protein.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

In certain embodiments, the binding agents can be used in any one of a number of conjugated (i.e. an immunoconjugate or radioconjugate) or non-conjugated forms. In certain embodiments, antibodies can be used in a non-conjugated form to harness the subject's natural defense mechanisms including complement-dependent cytotoxicity and antibody dependent cellular toxicity to eliminate the malignant or cancer cells.

In some embodiments, the binding agent is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi. In some embodiments, conjugates of an antibody and one or more small molecule toxins, such as a calicheamicin, maytansinoids, a trichothene, and CC1065, and the derivatives of these toxins that have toxin activity, can be produced. In certain embodiments, conjugates of an antibody and a cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In certain embodiments, the RSPO-LGR pathway inhibitor (e.g., antibody or soluble receptor) is an antagonist of at least one RSPO protein (i.e., 1, 2, 3, or 4 RSPO proteins). In certain embodiments, the RSPO-LGR pathway inhibitor inhibits activity of the RSPO protein(s) to which it binds. In certain embodiments, the RSPO-LGR pathway inhibitor inhibits at least about 10%, at least about 20%, at least about 30%, at least about 50%, at least about 75%, at least about 90%, or about 100% of the activity of the human RSPO protein(s) to which it binds. In certain embodiments, the RSPO-LGR pathway inhibitor inhibits activity of RSPO3.

In certain embodiments, the RSPO-LGR pathway inhibitor (e.g., antibody or soluble receptor) inhibits binding of at least one human RSPO to an appropriate receptor. In certain embodiments, the RSPO-LGR pathway inhibitor inhibits binding of at least one human RSPO protein to one or more human LGR proteins. In some embodiments, the at least one RSPO protein is selected from the group consisting of: RSPO1, RSPO2, RSPO3, and RSPO4. In some embodiments, the at least one RSPO protein is RSPO3. In some embodiments, the one or more human LGR proteins are selected from the group consisting of: LGR4, LGR5, and LGR6. In certain embodiments, the RSPO-LGR pathway inhibitor inhibits binding of one or more RSPO proteins to LGR4, LGR5, and/or LGR6. In certain embodiments, the inhibition of binding of a particular RSPO to a LGR protein by a RSPO-LGR pathway inhibitor is at least about 10%, at least about 25%, at least about 50%, at least about 75%, at least about 90%, or at least about 95%. In certain embodiments, a RSPO-LGR pathway inhibitor that inhibits binding of a RSPO to a LGR protein also inhibits RSPO-LGR pathway signaling. In certain embodiments, a RSPO-LGR pathway inhibitor that inhibits human RSPO pathway signaling is an antibody. In certain embodiments, a RSPO-LGR pathway inhibitor that inhibits human RSPO-LGR pathway signaling is an anti-RSPO antibody. In certain embodiments, a RSPO-LGR pathway inhibitor that inhibits human RSPO-LGR pathway signaling is an anti-RSPO3 antibody. In certain embodiments, a RSPO-LGR pathway inhibitor that inhibits human RSPO-LGR pathway signaling is OMP-131R010. In certain embodiments, a RSPO-LGR pathway inhibitor that inhibits human RSPO-LGR pathway signaling is an antibody comprising the 6 CDRs of OMP-131R010. In certain embodiments, a RSPO-LGR pathway inhibitor that inhibits human RSPO-LGR pathway signaling is an anti-LGR antibody. In certain embodiments, a RSPO-LGR pathway inhibitor that inhibits human RSPO-LGR pathway signaling is a LGR-Fc soluble receptor. In certain embodiments, a RSPO-LGR pathway inhibitor that inhibits human RSPO-LGR pathway signaling is a LGR5-Fc soluble receptor. In certain embodiments, the LGR5-Fc soluble receptor comprises amino acid sequence of SEQ ID NO:57. In certain embodiments, the LGR5-Fc soluble receptor comprises the amino acid sequence of SEQ ID NO:63.

In certain embodiments, the RSPO-LGR pathway inhibitors (e.g., antibody or soluble receptor) described herein are antagonists of at least one human RSPO protein and inhibit RSPO activity. In certain embodiments, the RSPO-LGR pathway inhibitor inhibits RSPO activity by at least about 10%, at least about 20%, at least about 30%, at least about 50%, at least about 75%, at least about 90%, or about 100%. In some embodiments, the RSPO-LGR pathway inhibitor inhibits activity of one, two, three, or four RSPO proteins. In some embodiments, the RSPO-LGR pathway inhibitor inhibits activity of at least one human RSPO protein selected from the group consisting of: RSPO1, RSPO2, RSPO3, and RSPO4. In some embodiments, the RSPO-binding agent binds at least RSPO3. In certain embodiments, a RSPO-LGR pathway inhibitor that inhibits human RSPO activity is an antibody. In certain embodiments, a RSPO-LGR pathway inhibitor that inhibits human RSPO activity is an anti-RSPO antibody. In certain embodiments, a RSPO-LGR pathway inhibitor that inhibits human RSPO activity is an anti-RSPO3 antibody. In certain embodiments, a RSPO-LGR pathway inhibitor that inhibits human RSPO activity is OMP-131R010. In certain embodiments, a RSPO-LGR pathway inhibitor that inhibits human RSPO activity is an antibody comprising the 6 CDRs of OMP-131R010. In certain embodiments, a RSPO-LGR pathway inhibitor that inhibits human RSPO activity is a LGR-Fc soluble receptor. In certain embodiments, a RSPO-LGR pathway inhibitor that inhibits human RSPO activity is a LGR5-Fc soluble receptor. In certain embodiments, the LGR5-Fc soluble receptor comprises amino acid sequence of SEQ ID NO:57. In certain embodiments, the LGR5-Fc soluble receptor comprises the amino acid sequence of SEQ ID NO:63.

In certain embodiments, the RSPO-LGR pathway inhibitor described herein is an antagonist of at least one human LGR protein and inhibits LGR activity. In certain embodiments, the RSPO-LGR pathway inhibitor inhibits LGR activity by at least about 10%, at least about 20%, at least about 30%, at least about 50%, at least about 75%, at least about 90%, or about 100%. In some embodiments, the RSPO-LGR pathway inhibitor inhibits activity of at least one human LGR protein selected from the group consisting of: LGR4, LGR5, and LGR6. In certain embodiments, the RSPO-LGR pathway inhibitor inhibits activity of LGR5. In some embodiments, the RSPO-LGR pathway inhibitor is an anti-LGR antibody. In certain embodiments, the RSPO-LGR pathway inhibitor is anti-LGR antibody comprising the 3 heavy chain CDRs of 88M1, and/or the 3 light chain CDRs of 88M1. In some embodiments, the anti-LGR antibody comprises the heavy chain variable region of 88M1, and/or the light chain variable region of 88M1.

In certain embodiments, the RSPO-LGR pathway inhibitor described herein is an antagonist of at least one human RSPO protein and inhibits RSPO signaling. In certain embodiments, the RSPO-LGR pathway inhibitor inhibits RSPO signaling by at least about 10%, at least about 20%, at least about 30%, at least about 50%, at least about 75%, at least about 90%, or about 100%. In some embodiments, the RSPO-LGR pathway inhibitor inhibits signaling by one, two, three, or four RSPO proteins. In some embodiments, the RSPO-LGR pathway inhibitor inhibits signaling of at least one RSPO protein selected from the group consisting of RSPO1, RSPO2, RSPO3, and RSPO4. In some embodiments, the RSPO-LGR pathway inhibitor inhibits signaling of at least RSPO3. In certain embodiments, a RSPO-LGR pathway inhibitor that inhibits RSPO signaling is an antibody. In certain embodiments, a RSPO-LGR pathway inhibitor that inhibits RSPO signaling is an anti-RSPO antibody. In certain embodiments, a RSPO-LGR pathway inhibitor that inhibits RSPO signaling is an anti-RSPO3 antibody. In certain embodiments, a RSPO-LGR pathway inhibitor that inhibits RSPO signaling is OMP-131R010. In certain embodiments, a RSPO-LGR pathway inhibitor that inhibits RSPO signaling is an antibody comprising the 6 CDRs of OMP-131R010. In certain embodiments, a RSPO-LGR pathway inhibitor that inhibits RSPO signaling is a soluble receptor. In certain embodiments, a RSPO-LGR pathway inhibitor that inhibits RSPO signaling is a LGR-Fc soluble receptor. In certain embodiments, a RSPO-LGR pathway inhibitor that inhibits RSPO signaling is a LGR5-Fc soluble receptor. In certain embodiments, the LGR5-Fc soluble receptor comprises amino acid sequence of SEQ ID NO:57. In certain embodiments, the LGR5-Fc soluble receptor comprises the amino acid sequence of SEQ ID NO:63.

In certain embodiments, a RSPO-LGR pathway inhibitor described herein is an antagonist of β-catenin signaling. In certain embodiments, the RSPO-LGR pathway inhibitor inhibits β-catenin signaling by at least about 10%, at least about 20%, at least about 30%, at least about 50%, at least about 75%, at least about 90%, or about 100%. In certain embodiments, a RSPO-LGR pathway inhibitor that inhibits β-catenin signaling is an antibody. In certain embodiments, a RSPO-LGR pathway inhibitor that inhibits β-catenin signaling is an anti-RSPO antibody. In certain embodiments, a RSPO-LGR pathway inhibitor that inhibits β-catenin signaling is an anti-RSPO3 antibody. In certain embodiments, a RSPO-LGR pathway inhibitor that inhibits β-catenin signaling is OMP-131R010. In certain embodiments, a RSPO-LGR pathway inhibitor that inhibits β-catenin signaling is an antibody comprising the 6 CDRs of OMP-131R010. In certain embodiments, a RSPO-LGR pathway inhibitor that inhibits β-catenin signaling is an anti-LGR antibody. In certain embodiments, a RSPO-LGR pathway inhibitor that inhibits β-catenin signaling is an anti-LGR antibody comprising the 3 heavy chain CDRs of 88M1, and/or the 3 light chain CDRs of 88M1. In some embodiments, the anti-LGR antibody comprises the heavy chain variable region of 88M1, and/or the light chain variable region of 88M1. In certain embodiments, a RSPO-LGR pathway inhibitor that inhibits β-catenin signaling is a soluble receptor. In certain embodiments, a RSPO-LGR pathway inhibitor that inhibits β-catenin signaling is a LGR-Fc soluble receptor. In certain embodiments, a RSPO-LGR pathway inhibitor that inhibits β-catenin signaling is a LGR5-Fc soluble receptor. In certain embodiments, the LGR5-Fc soluble receptor comprises amino acid sequence of SEQ ID NO:57. In certain embodiments, the LGR5-Fc soluble receptor comprises the amino acid sequence of SEQ ID NO:63.

In certain embodiments, the RSPO-LGR pathway inhibitor described herein inhibits binding of at least one RSPO protein to a receptor. In certain embodiments, the RSPO-LGR pathway inhibitor inhibits binding of at least one human RSPO protein to one or more of its receptors. In some embodiments, the RSPO-LGR pathway inhibitor inhibits binding of at least one RSPO protein to at least one LGR protein. In some embodiments, the RSPO-binding agent inhibits binding of at least one RSPO protein to LGR4, LGR5, and/or LGR6. In certain embodiments, the inhibition of binding of at least one RSPO to at least one LGR protein is at least about 10%, at least about 25%, at least about 50%, at least about 75%, at least about 90%, or at least about 95%. In certain embodiments, a RSPO-LGR pathway inhibitor that inhibits binding of at least one RSPO to at least one LGR protein further inhibits RSPO-LGR pathway signaling and/or β-catenin signaling. In certain embodiments, a RSPO-LGR pathway inhibitor that inhibits binding of at least one human RSPO to at least one LGR protein is an antibody. In certain embodiments, a RSPO-LGR pathway inhibitor that inhibits binding of at least one human RSPO to at least one LGR protein is an anti-LGR antibody. In certain embodiments, a RSPO-LGR pathway inhibitor that inhibits binding of at least one human RSPO to at least one LGR protein is an anti-LGR antibody comprising the 3 heavy chain CDRs of 88M1, and/or the 3 light chain CDRs of 88M1. In some embodiments, the anti-LGR antibody comprises the heavy chain variable region of 88M1 and/or the light chain variable region of 88M1. In certain embodiments, a RSPO-LGR pathway inhibitor that inhibits binding of at least one human RSPO to at least one LGR protein is a soluble receptor. In certain embodiments, a RSPO-LGR pathway inhibitor that inhibits binding of at least one human RSPO to at least one LGR protein is a LGR-Fc soluble receptor. In certain embodiments, a RSPO-LGR pathway inhibitor that inhibits binding of at least one human RSPO to at least one LGR protein is a LGR5-Fc soluble receptor. In certain embodiments, the LGR5-Fc soluble receptor comprises amino acid sequence of SEQ ID NO:57. In certain embodiments, the LGR5-Fc soluble receptor comprises the amino acid sequence of SEQ ID NO:63.

In certain embodiments, the RSPO-LGR pathway inhibitor described herein blocks binding of at least one RSPO to a receptor. In certain embodiments, the RSPO-LGR pathway inhibitor blocks binding of at least one human RSPO protein to one or more of its receptors. In some embodiments, the RSPO-LGR pathway inhibitor blocks binding of at least one RSPO to at least one LGR protein. In some embodiments, the RSPO-LGR pathway inhibitor blocks binding of at least one RSPO protein to LGR4, LGR5, and/or LGR6. In certain embodiments, the blocking of binding of at least one RSPO to at least one LGR protein is at least about 10%, at least about 25%, at least about 50%, at least about 75%, at least about 90%, or at least about 95%. In certain embodiments, a RSPO-LGR pathway inhibitor that blocks binding of at least one RSPO protein to at least one LGR protein further inhibits RSPO-LGR pathway signaling and/or β-catenin signaling. In certain embodiments, a RSPO-LGR pathway inhibitor that blocks binding of at least one human RSPO to at least one LGR protein is an antibody. In certain embodiments, a RSPO-LGR pathway inhibitor that blocks binding of at least one human RSPO to at least one LGR protein is an anti-LGR antibody. In certain embodiments, a RSPO-LGR pathway inhibitor that blocks binding of at least one human RSPO to at least one LGR protein is an anti-LGR antibody comprising the 3 heavy chain CDRs of 88M1 and/or the 3 light chain CDRs of 88M1. In some embodiments, the anti-LGR antibody comprises the heavy chain variable region of 88M1 and/or the light chain variable region of 88M1. In certain embodiments, a RSPO-LGR pathway inhibitor that blocks binding of at least one human RSPO to at least one LGR protein is a soluble receptor. In certain embodiments, a RSPO-LGR pathway inhibitor that blocks binding of at least one human RSPO to at least one LGR protein is a LGR-Fc soluble receptor. In certain embodiments, a RSPO-LGR pathway inhibitor that blocks binding of at least one human RSPO to at least one LGR protein is a LGR5-Fc soluble receptor. In certain embodiments, the LGR5-Fc soluble receptor comprises amino acid sequence of SEQ ID NO:57. In certain embodiments, the LGR5-Fc soluble receptor comprises the amino acid sequence of SEQ ID NO:63.

In certain embodiments, the RSPO-LGR pathway inhibitor described herein inhibits RSPO pathway signaling. It is understood that a RSPO-LGR pathway inhibitor that inhibits RSPO-LGR pathway signaling can, in certain embodiments, inhibit signaling by one or more receptors in the RSPO-LGR signaling pathway but not necessarily inhibit signaling by all receptors. In certain alternative embodiments, RSPO pathway signaling by all human receptors can be inhibited. In certain embodiments, RSPO pathway signaling by one or more receptors selected from the group consisting of LGR4, LGR5, and LGR6 is inhibited. In certain embodiments, the inhibition of RSPO-LGR pathway signaling by a RSPO-LGR pathway inhibitor is a reduction in the level of RSPO-LGR pathway signaling of at least about 10%, at least about 25%, at least about 50%, at least about 75%, at least about 90%, or at least about 95%. In some embodiments, a RSPO-LGR pathway inhibitor that inhibits RSPO-LGR pathway signaling is an antibody. In some embodiments, a RSPO-LGR pathway inhibitor that inhibits RSPO-LGR pathway signaling is an anti-LGR antibody. In some embodiments, a RSPO-LGR pathway inhibitor that inhibits RSPO-LGR pathway signaling is an anti-LGR antibody comprising the 3 heavy chain CDRs of 88M1 and/or the 3 light chain CDRs of 88M1. In some embodiments, the anti-LGR antibody comprises the heavy chain variable region of 88M1 and/or the light chain variable region of 88M1. In some embodiments, a RSPO-LGR pathway inhibitor that inhibits RSPO-LGR pathway signaling is a soluble receptor. In some embodiments, a RSPO-LGR pathway inhibitor that inhibits RSPO-LGR pathway signaling is a LGR-Fc soluble receptor. In some embodiments, a RSPO-LGR pathway inhibitor that inhibits RSPO-LGR pathway signaling is a LGR5-Fc soluble receptor. In certain embodiments, the LGR5-Fc soluble receptor comprises amino acid sequence of SEQ ID NO:57. In certain embodiments, the LGR5-Fc soluble receptor comprises the amino acid sequence of SEQ ID NO:63.

In certain embodiments, the RSPO-LGR pathway inhibitor described herein inhibits activation of β-catenin. It is understood that a RSPO-LGR pathway inhibitor that inhibits activation of β-catenin can, in certain embodiments, inhibit activation of β-catenin by one or more receptors, but not necessarily inhibit activation of β-catenin by all receptors. In certain alternative embodiments, activation of β-catenin by all human receptors can be inhibited. In certain embodiments, activation of β-catenin by one or more receptors selected from the group consisting of LGR4, LGR5, and LGR6 is inhibited. In certain embodiments, the inhibition of activation of β-catenin by a RSPO-binding agent or LGR-binding agent is a reduction in the level of activation of β-catenin of at least about 10%, at least about 25%, at least about 50%, at least about 75%, at least about 90%, or at least about 95%. In some embodiments, a RSPO-LGR pathway inhibitor that inhibits activation of β-catenin is an antibody. In some embodiments, a RSPO-LGR pathway inhibitor that inhibits activation of β-catenin is an anti-LGR antibody. In some embodiments, a RSPO-LGR pathway inhibitor that inhibits activation of β-catenin is an anti-LGR antibody comprising the 3 heavy chain CDRs of 88M1 and/or the 3 light chain CDRs of 88M1. In some embodiments, the anti-LGR antibody comprises the heavy chain variable region of 88M1 and/or the light chain variable region of 88M1. In some embodiments, a RSPO-LGR pathway inhibitor that inhibits activation of β-catenin is a soluble receptor. In some embodiments, a RSPO-LGR pathway inhibitor that inhibits activation of β-catenin is a LGR-Fc soluble receptor. In some embodiments, a RSPO-LGR pathway inhibitor that inhibits activation of μ-catenin is a LGR5-Fc soluble receptor. In certain embodiments, the LGR5-Fc soluble receptor comprises amino acid sequence of SEQ ID NO:57. In certain embodiments, the LGR5-Fc soluble receptor comprises the amino acid sequence of SEQ ID NO:63.

In certain embodiments, a RSPO-LGR pathway inhibitor has one or more of the following effects: inhibit proliferation of tumor cells, inhibit tumor growth, reduce the frequency of cancer stem cells in a tumor, reduce the tumorigenicity of a tumor, reduce the tumorigenicity of a tumor by reducing the frequency of cancer stem cells in the tumor, trigger cell death of tumor cells, induce cells in a tumor to differentiate, differentiate tumorigenic cells to a non-tumorigenic state, induce expression of differentiation markers in the tumor cells, prevent metastasis of tumor cells, or decrease survival of tumor cells.

In certain embodiments, a RSPO-LGR pathway inhibitor is capable of inhibiting tumor growth and/or reducing tumor size. In certain embodiments, a RSPO-LGR pathway inhibitor is capable of inhibiting tumor growth and/or reducing tumor size in vivo (e.g., in a xenograft mouse model and/or in a human having cancer). In some embodiments, the tumor is a tumor selected from the group consisting of colorectal tumor, colon tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In certain embodiments, the tumor is a breast tumor. In certain embodiments, the tumor is an ovarian tumor. In certain embodiments, the tumor is a lung tumor. In certain embodiments, the tumor is a pancreatic tumor. In certain embodiments, the tumor is a RSPO-dependent tumor, LGR-dependent tumor, or β-catenin-dependent tumor.

In certain embodiments, a RSPO-LGR pathway inhibitor is capable of reducing the tumorigenicity of a tumor. In certain embodiments, a RSPO-LGR pathway inhibitor is capable of reducing the tumorigenicity of a tumor comprising cancer stem cells in an animal model, such as a mouse xenograft model. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the number or frequency of cancer stem cells is determined by limiting dilution assay using an animal model. Additional examples and guidance regarding the use of limiting dilution assays to determine a reduction in the number or frequency of cancer stem cells in a tumor can be found, e.g., in International Publication Number WO 2008/042236, and U.S. Patent Publication Nos. 2008/0064049, and 2008/0178305, each of which is hereby incorporated by reference herein in its entirety for all purposes.

In certain embodiments, a RSPO-LGR pathway inhibitor is active in vivo for at least 1 hour, at least about 2 hours, at least about 5 hours, at least about 10 hours, at least about 24 hours, at least about 2 days, at least about 3 days, at least about 1 week, or at least about 2 weeks. In certain embodiments, the RSPO-LGR pathway inhibitor is an IgG (e.g., IgG1 or IgG2) antibody that is active in vivo for at least 1 hour, at least about 2 hours, at least about 5 hours, at least about 10 hours, at least about 24 hours, at least about 2 days, at least about 3 days, at least about 1 week, or at least about 2 weeks. In certain embodiments, the RSPO-LGR pathway inhibitor is a fusion protein that is active in vivo for at least 1 hour, at least about 2 hours, at least about 5 hours, at least about 10 hours, at least about 24 hours, at least about 2 days, at least about 3 days, at least about 1 week, or at least about 2 weeks.

In certain embodiments, a RSPO-LGR pathway inhibitor has a circulating half-life in mice, cynomolgus monkeys, or humans of at least about 5 hours, at least about 10 hours, at least about 24 hours, at least about 2 days, at least about 3 days, at least about 1 week, or at least about 2 weeks. In certain embodiments, the RSPO-LGR pathway inhibitor is an IgG (e.g., IgG1 or IgG2) antibody that has a circulating half-life in mice, cynomolgus monkeys, or humans of at least about 5 hours, at least about 10 hours, at least about 24 hours, at least about 2 days, at least about 3 days, at least about 1 week, or at least about 2 weeks. In certain embodiments, the RSPO-LGR pathway inhibitor is a fusion protein that has a circulating half-life in mice, cynomolgus monkeys, or humans of at least about 5 hours, at least about 10 hours, at least about 24 hours, at least about 2 days, at least about 3 days, at least about 1 week, or at least about 2 weeks. Methods of increasing (or decreasing) the half-life of agents such as polypeptides and antibodies are known in the art. For example, known methods of increasing the circulating half-life of IgG antibodies include the introduction of mutations in the Fc region which increase the pH-dependent binding of the antibody to the neonatal Fc receptor (FcRn). Known methods of increasing the circulating half-life of antibody fragments lacking the Fc region include such techniques as PEGylation.

### IV. Mitotic Inhibitors

Described herein are methods for inhibiting tumor growth, for reducing tumor size, and/or for the treatment of cancer, the methods comprising administering a RSPO-LGR pathway inhibitor in combination with mitotic inhibitors. Mitotic inhibitors or anti-mitotic agents include, but are not limited to, microtubule binders, microtubule enzyme inhibitors, mitosis checkpoint kinase (CHK) inhibitors, and mitosis enzyme inhibitors. Microtubule binders, include but are not limited to, taxanes, taxoids, vinca alkaloids, alkaloids, epothilones, and halichondrins.

In some embodiments, a mitotic inhibitor is selected from the group consisting of a taxane, a vinca alkaloid, an epothilone, or a halichondrin. In some embodiments, a mitotic inhibitor is a taxane. Taxanes induce a mitotic cell-cycle block through the inhibition of microtubule depolymerization (i.e., stabilization of the microtubule polymers). The mitotic cell-cycle block results in mitotic arrest and apoptosis. In some embodiments, a taxane is selected from the group consisting of: paclitaxel (TAXOL), nab-paclitaxel (ABRAXANE), docetaxel (TAXOTERE), cabazitaxel (JEVTANA), tesetaxel, larotaxel, ortataxel, DHA-paclitaxel, PG-paclitaxel, and pharmaceutically acceptable salts, acids, or derivatives thereof. In some embodiments, the taxane is paclitaxel. In some embodiments, the taxane is nab-paclitaxel. In some embodiments, the mitotic inhibitor is a vinca alkaloid. In some embodiments, the vinca alkaloid is selected from the group consisting of vinblastine (VELBAN), vincristine (MARQIBO), vinorelbine (NAVELBINE), vincadifformine, vindesine, vinflunine, minovincine, and pharmaceutically acceptable salts, acids, or derivatives thereof. In some embodiments, the mitotic inhibitor is an alkaloid such as neoxaline. In some embodiments, the mitotic inhibitor is an epothilone. In some embodiments, the epothilone is ixabepilone (IXEMPRA). In some embodiments, the mitotic inhibitor is halichondrin B. In some embodiments, the halichondrin is analogue eribulin mesylate (HALAVEN). In some embodiments, the mitotic inhibitor is a microtubule enzyme inhibitor. In some embodiments, the microtubule enzyme inhibitor is selected from the group consisting of ARQ 621, EMD 534085, and LY2523355. In some embodiments, the mitotic inhibitor is a mitosis checkpoint kinase inhibitor. In some embodiments, the mitosis checkpoint kinase inhibitor is LY2603618. In some embodiments, the mitotic inhibitor is a mitosis enzyme inhibitor. In some embodiments, the mitosis enzyme inhibitor is an inhibitor of Aurora A or PLK1. In some embodiments, the mitosis enzyme inhibitor is selected from the group consisting of MLN8237, ENMD-0276, AZD1152, GSK1070916A, PHA-739358, SNS-314, CYC116, PF-03814735, AT9238, AS703569, and BI 6727.

### EXAMPLES

### Example 1

Activity of Anti-RSPO3 Antibody OMP-131R010 in Combination with a Chemotherapeutic Agent in In Vivo Ovarian Tumor Model

OncoMed xenograft models described herein were established at OncoMed Pharmaceuticals from minimally passaged, patient-derived tumor specimens. The tumor specimens were examined by a pathologist and classified as a specific tumor type. OncoMed relies on these classifications unless further analyses are done on any specific tumor and a reclassification is deemed necessary.

Single cell suspensions of xenograft OMP-OV19 ovarian tumor cells (1×10⁵ cells) were injected subcutaneously into NOD/SCID mice. Tumors were allowed to grow 39 days until they reached an average volume of approximately 120 mm³. Tumor-bearing mice were randomized into four groups (n=8-9 animals per group). Tumor-bearing mice were treated with either (i) control antibody, (ii) paclitaxel alone, (iii) anti-RSPO3 antibody OMP-131R010 plus paclitaxel dosed on the same day, or (iv) anti-RSPO3 antibody OMP-131R010 plus paclitaxel where the antibody was administered two days prior to the paclitaxel. Antibodies were dosed at 25 mg/kg and administered every other week. Paclitaxel was dosed at 20 mg/kg and administered every other week. Tumor volumes were measured on the indicated days post-treatment and are shown as the mean±SEM in FIG. 1A. Tumor volumes of the individual animals in the two combination treatment groups on Day 61 are shown in FIG. 1B. Surprisingly, the staggered administration of OMP-131R010 and paclitaxel, where OMP-131R010 is administered prior to administration of paclitaxel, resulted in greater tumor growth inhibition than dosing on the same day. Indeed, staggered administration of OMP-131R010 and paclitaxel not only inhibited tumor growth, but actually decreased tumor size over the course of the treatment.

### Example 2

Activity of Anti-RSPO3 Antibody OMP-131R010 in Combination with a Chemotherapeutic Agent in In Vivo Lung Cancer Model

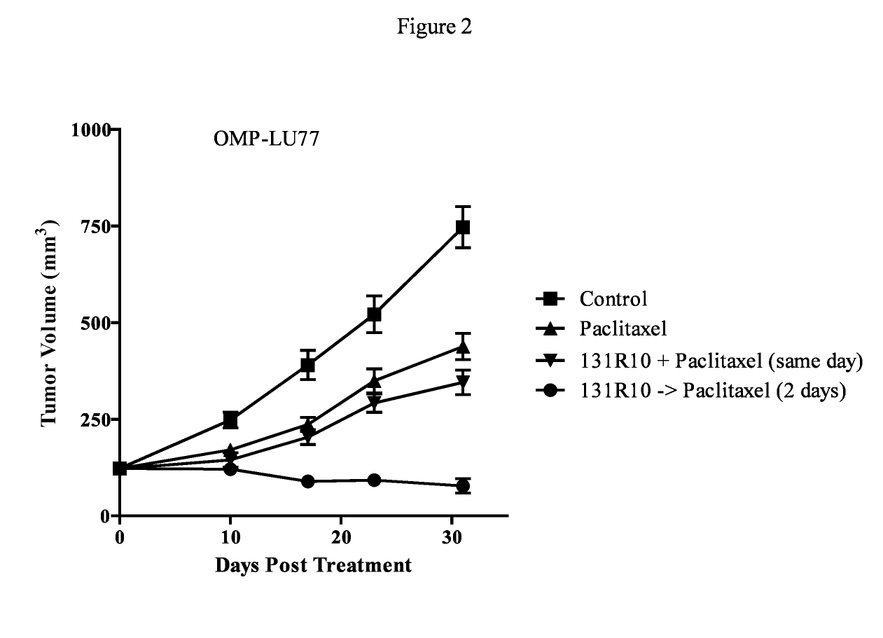

Single cell suspensions of xenograft OMP-LU77 lung tumor cells (5×10⁴ cells) were injected subcutaneously into NOD/SCID mice. Tumors were allowed to grow 34 days until they reached an average volume of approximately 125 mm³. Tumor-bearing mice were randomized into four groups (n=9 animals per group). Tumor-bearing mice were treated with (i) control antibody, (ii) paclitaxel alone, (iii) anti-RSPO3 antibody OMP-131R010 plus paclitaxel dosed on the same day, or (iv) anti-RSPO3 antibody OMP-131R010 plus paclitaxel where the antibody was administered two days prior to the paclitaxel. Antibodies were dosed at 25 mg/kg, paclitaxel was dosed at 20 mg/kg, and both agents were administered once every three weeks. Tumor volumes were measured on the indicated days post-treatment. Results are shown in FIG. 2. As seen with the ovarian tumor model in Example 1, the staggered administration of OMP-131R010 and paclitaxel, where OMP-131R010 is administered prior to administration of paclitaxel, resulted in greater tumor growth inhibition than dosing on the same day. Indeed, staggered administration of OMP-131R010 and paclitaxel not only inhibited tumor growth, but actually decreased tumor size over the course of the treatment.

### Example 3

Activity of Anti-RSPO3 Antibody OMP-131R010 in Combination with a Chemotherapeutic Agent in In Vivo Colorectal Cancer Model

Single cell suspensions of xenograft OMP-C8 colon tumor cells (5×10⁴ cells) were injected subcutaneously into NOD/SCID mice. OMP-C8 colon tumor cells comprise an inactivating mutation in the APC gene and express low levels of RSPO3 (data not shown). Tumors were allowed to grow 23 days until they reached an average volume of approximately 100 mm³. Tumor-bearing mice were randomized into four groups (n=9 animals per group). Tumor-bearing mice were treated with (i) control antibody, (ii) nab-paclitaxel (ABRAXANE) alone, (iii) anti-RSPO3 antibody OMP-131R010 plus nab-paclitaxel dosed on the same day, (iv) anti-RSPO3 antibody OMP-131R010 plus nab-paclitaxel where the antibody was administered two days prior to paclitaxel, (v) fluorouracil and irinotecan, (vi) anti-RSPO3 antibody OMP-131R010 plus fluorouracil and irinotecan dosed on the same day, or (vii) anti-RSPO3 antibody OMP-131R010 plus fluorouracil and irinotecan where the antibody was administered two days prior to fluorouracil and irinotecan. Antibodies were dosed weekly at 25 mg/kg, nab-paclitaxel was dosed at 30 mg/kg, fluorouracil was dosed at 50 mg/kg, and irinotecan was dosed at 5 mg/kg. Antibodies were administered every other week and chemotherapy was administered weekly. Tumor volumes were measured on the indicated days post-treatment.

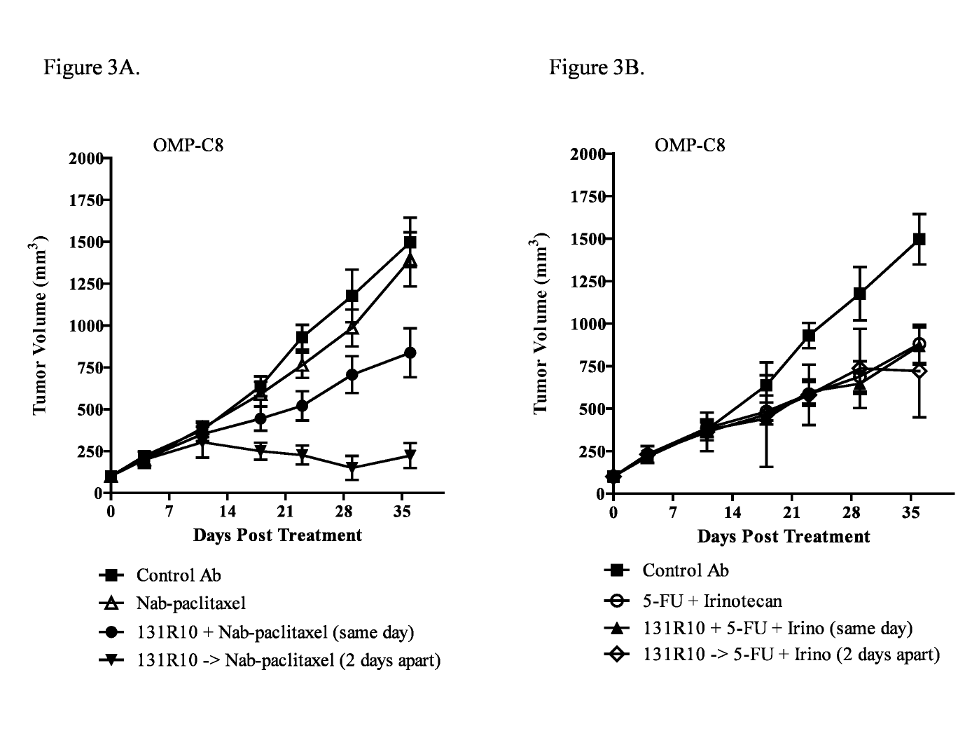

Results are shown in FIGS. 3A and 3B. The staggered administration of OMP-131R010 and nab-paclitaxel, where OMP-131R010 is administered prior to administration of nab-paclitaxel, resulted in greater tumor growth inhibition than dosing on the same day. In contrast, staggered administration of OMP-131R010 in combination with fluorouracil and irinotecan did not result in greater tumor growth inhibition than dosing on the same day. Furthermore, the combination of OMP-131R010 and nab-paclitaxel administered in a sequential manner had greater efficacy in inhibiting tumor growth than the combination of OMP-131R010 with a chemotherapeutic agent that is not a mitotic inhibitor.

These studies suggest that the order of dosing can have a significant impact on the extent of tumor growth inhibition, particularly in cases where the RSPO-LGR pathway inhibitor and the paclitaxel are administered sequentially. In addition, these studies suggest that taxanes in combination with an anti-RSPO3 antibody may be a new treatment option for colon cancer, as taxanes, in general, are not considered a standard-of-care therapeutic agent for colon or colorectal cancer treatment.

It is understood that the examples and embodiments described herein are for illustrative purposes only and that various modifications or changes in light thereof will be suggested to person skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

Following are the sequences disclosed in the application:

Human RSPO1 amino acid sequence with signal sequence
(SEQ ID NO: 1)
MRLGLCVVALVLSWTHLTISSRGIKGKRQRRISAEGSQACAKGCELCSEVNGCLKCSPKL
FILLERNDIRQVGVCLPSCPPGYFDARNPDMNKCIKCKIEHCEACFSHNECTKCKEGLYL
HKGRCYPACPEGSSAANGTMECSSPAQCEMSEWSPWGPCSKKQQLCGFRRGSEERTRRVL
HAPVGDHAACSDTKETRRCTVRRVPCPEGQKRRKGGQGRRENANRNLARKESKEAGAGSR
RRKGQQQQQQQGTVGPLTSAGPA
Human RSPO2 amino acid sequence with signal sequence
(SEQ ID NO: 2)
MQFRLFSFALIILNCMDYSHCQGNRWRRSKRASYVSNPICKGCLSCSKDNGCSRCQQKLF
FFLRREGMRQYGECLHSCPSGYYGHRAPDMNRCARCRIENCDSCFSKDFCTKCKVGFYLH
RGRCFDECPDGFAPLEETMECVEGCEVGHWSEWGTCSRNNRTCGFKWGLETRTRQIVKKP
VKDTIPCPTIAESRRCKMTMRHCPGGKRTPKAKEKRNKKKKRKLIERAQEQHSVFLATDR
ANQ
Human RSPO3 amino acid sequence with signal sequence
(SEQ ID NO: 3)
MHLRLISWLFIILNFMEYIGSQNASRGRRQRRMHPNVSQGCQGGCATCSDYNGCLSCKPR
LFFALERIGMKQIGVCLSSCPSGYYGTRYPDINKCTKCKADCDTCFNKNFCTKCKSGFYL
HLGKCLDNCPEGLEANNHTMECVSIVHCEVSEWNPWSPCTKKGKTCGFKRGTETRVREII
QHPSAKGNLCPPTNETRKCTVQRKKCQKGERGKKGRERKRKKPNKGESKEAIPDSKSLES
SKEIPEQRENKQQQKKRKVQDKQKSVSVSTVH
Human RSPO4 amino acid sequence with signal sequence
(SEQ ID NO: 4)
MRAPLCLLLLVAHAVDMLALNRRKKQVGTGLGGNCTGCIICSEENGCSTCQQRLFLFIRR
EGIRQYGKCLHDCPPGYFGIRGQEVNRCKKCGATCESCFSQDFCIRCKRQFYLYKGKCLP
TCPPGTLAHQNTRECQGECELGPWGGWSPCTHNGKTCGSAWGLESRVREAGRAGHEEAAT
CQVLSESRKCPIQRPCPGERSPGQKKGRKDRRPRKDRKLDRRLDVRPRQPGLQP
89M5 Heavy chain CDR1
(SEQ ID NO: 5)
TGYTMH
89M5 Heavy chain CDR2
(SEQ ID NO: 6)
GINPNNGGTTYNQNFKG
89M5 Heavy chain CDR3
(SEQ ID NO: 7)
KEFSDGYYFFAY
89M5 Light chain CDR1
(SEQ ID NO: 8)
KASQDVIFAVA
89M5 Light chain CDR2
(SEQ ID NO: 9)
WASTRHT
89M5 Light chain CDR3
(SEQ ID NO: 10)
QQHYSTPW
h89M5-H8L5 Heavy chain variable region amino acid sequence
(SEQ ID NO: 11)
EVQLVQSGAEVKKPGESLRISCKGSGYSFTGYTMHWVRQMPGKGLEWMGGINPNNGGTTY
NQNFKGHVTISADKSISTAYLQWSSLKASDTAMYYCARKEFSDGYYFFAYWGQGTLVTVS
S
h89M5-H8L5 Light chain variable region amino acid sequence
(SEQ ID NO: 12)
DIVMTQSPDSLAVSLGERATINCKASQDVIFAVAWYQQKPGQPPKLLIYWASTRHTGVPD
RFSGSGSGTDFTLTISSLQAEDVAVYYCQQHYSTPWTFGGGTKVEIK
h89M5-H8L5 Heavy chain amino acid sequence with predicted signal
sequence underlined
(SEQ ID NO: 13)
MDWTWRILFLVAAATGAHSEVQLVQSGAEVKKPGESLRISCKGSGYSFTGYTMHWVRQMP
GKGLEWMGGINPNNGGTTYNQNFKGHVTISADKSISTAYLQWSSLKASDTAMYYCARKEF
SDGYYFFAYWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKRVEP
KSCDKTHTCPPCPAPELLGGPSVFLEPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
h89M5-H8L5 Heavy chain amino acid sequence without predicted signal
sequence
(SEQ ID NO: 14)
EVQLVQSGAEVKKPGESLRISCKGSGYSFTGYTMHWVRQMPGKGLEWMGGINPNNGGTTY
NQNFKGHVTISADKSISTAYLQWSSLKASDTAMYYCARKEFSDGYYFFAYWGQGTLVTVS
SASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTHTCPPCPAPELLG
GPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFLYSKLTVDKSR
WQQGNVFSCSVMHEALHNHYTQKSLSLSPGK
h89M5-H8L5 Light chain amino acid sequence with predicted signal
sequence underlined
(SEQ ID NO: 15)
MVLQTQVFISLLLWISGAYGDIVMTQSPDSLAVSLGERATINCKASQDVIFAVAWYQQKP
GQPPKLLIYWASTRHTGVPDRFSGSGSGTDFTLTISSLQAEDVAVYYCQQHYSTPWTFGG
GTKVEIKRTVAAPSVFIFPPSDEQLKSGTASVVCLLNNFYPREAKVQWKVDNALQSGNSQ
ESVTEQDSKDSTYSLSNTLTLSKADYEKHKVYACEVTHQGLSSPVTKSFNRGEC
h89M5-H8L5 Light chain amino acid sequence without predicted signal
sequence
(SEQ ID NO: 16)
DIVMTQSPDSLAVSLGERATINCKASQDVIFAVAWYQQKPGQPPKLLIYWASTRHTGVPD
RFSGSGSGTDFTLTISSLQAEDVAVYYCQQHYSTPWTFGGGTKVEIKRTVAAPSVFIFPP
SDEQLKSGTASVVCLLNNFYPREAKVQWKVDNALQSGNSQESVTEQDSKDSTYSLSNTLT
LSKADYEKHKVYACEVTHQGLSSPVTKSFNRGEC
130M23 Heavy chain CDR1
(SEQ ID NO: 17)
SSYAMS
130M23 Heavy chain CDR2
(SEQ ID NO: 18)
SISSGGSTYYPDSVKG
130M23 Heavy chain CDR3
(SEQ ID NO: 19)
RGGDPGVYNGDYEDAMDY
130M23 Light chain CDR1
(SEQ ID NO: 20)
KASQDVSSAVA
130M23 Light chain CDR2
(SEQ ID NO: 21)
WASTRHT
130M23 Light chain CDR3
(SEQ ID NO: 22)
QQHYSTP
h130M23-H1L6 Heavy chain variable region amino acid sequence
(SEQ ID NO: 23)
EVQLVESGGGLVKPGGSLRLSCAASGFTFSSYAMSWVRQAPGKGLEWVSSISSGGSTYYP
DSVKGRFTISRDNAKNSLYLQMNSLRAEDTAVYYCARGGDPGVYNGDYEDAMDYWGQGTT
VTVSS
h130M23-H1L6 Light chain variable region amino acid sequence
(SEQ ID NO: 24)
DIQMTQSPSSLSASVGDRVTITCKASQDVSSAVAWYQQKPGKAPKLLIYWASTRHTGVPS
RFSGSGSGTDFTLTISSLQPEDFATYYCQQHYSTPWTFGQGTKVEIK
h130M23-H1L6 Heavy chain amino acid sequence with predicted signal
sequence underlined
(SEQ ID NO: 25)
MELGLRWVELVAILEGVQCEVQLVESGGGLVKPGGSLRLSCAASGFTFSSYAMSWVRQAP
GKGLEWVSSISSGGSTYYPDSVKGRFTISRDNAKNSLYLQMNSLRAEDTAVYYCARGGDP
GVYNGDYEDAMDYWGQGTTVTVSSASTKGPSVFPLAPCSRSTSESTAALGCLVKDYFPEP
VTVSWNSGALTSGVHTFPAVLQSSGLYSLSSVVTVPSSNFGTQTYTCNVDHKPSNTKVDK
TVERKCCVECPPCPAPPVAGPSVFLFPPKPKDTLMISRTPEVTCVVVDVSHEDPEVQFNW
YVDGVEVHNAKTKPREEQFNSTFRVVSVLTVVHQDWLNGKEYKCKVSNKGLPAPIEKTIS
KTKGQPREPQVYTLPPSREEMTKNQVSLTCLVKGFYPSDIAVEWESNGQPENNYKTTPPM
LDSDGSFFLYSKLTVDKSRWQQGNVFSCSVMHEALHNHYTQKSLSLSPGK
h130M23-H1L6 Heavy chain amino acid sequence without predicted signal
sequence
(SEQ ID NO: 26)
EVQLVESGGGLVKPGGSLRLSCAASGFTFSSYAMSWVRQAPGKGLEWVSSISSGGSTYYP
DSVKGRFTISRDNAKNSLYLQMNSLRAEDTAVYYCARGGDPGVYNGDYEDAMDYWGQGTT
VTVSSASTKGPSVFPLAPCSRSTSESTAALGCLVKDYFPEPVTVSWNSGALTSGVHTFPA
VLQSSGLYSLSSVVTVPSSNFGTQTYTCNVDHKPSNTKVDKTVERKCCVECPPCPAPPVA
GPSVFLFPPKPKDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPREPQVYTLPPSRE
EMTKNQVSLTCLVKGFYPSDIAVEWESNGQPENNYKTTPPMLDSDGSFFLYSKLTVDKSR
WQQGNVFSCSVMHEALHNHYTQKSLSLSPGK
h130M23-H1L6 Light chain amino acid sequence with predicted signal
sequence underlined
(SEQ ID NO: 27)
MGIKMESQIQAFVFVFLWLSGVDGDIQMTQSPSSLSASVGDRVTITCKASQDVSSAVAWY
QQKPGKAPKLLIYWASTRHTGVPSRFSGSGSGTDFTLTISSLQPEDFATYYCQQHYSTPW
TFGQGTKVEIKRTVAAPSVFIFPPSDEQLKSGTASVVCLLNNFYPREAKVQWKVDNALQS
GNSQESVTEQDSKDSTYSLSNTLTLSKADYEKHKVYACEVTHQGLSSPVTKSFNRGEC
h130M23-H1L6 Light chain amino acid sequence without predicted signal
sequence
(SEQ ID NO: 28)
DIQMTQSPSSLSASVGDRVTITCKASQDVSSAVAWYQQKPGKAPKLLIYWASTRHTGVPS
RFSGSGSGTDFTLTISSLQPEDFATYYCQQHYSTPWTFGQGTKVEIKRTVAAPSVFIFPP
SDEQLKSGTASVVCLLNNFYPREAKVQWKVDNALQSGNSQESVTEQDSKDSTYSLSNTLT
LSKADYEKHKVYACEVTHQGLSSPVTKSFNRGEC
131R010 Heavy chain CDR1
(SEQ ID NO: 29)
DYSIH
131R010 Heavy chain CDR2
(SEQ ID NO: 30)
YIYPSNGDSGYNQKFK
131R010 Heavy chain CDR3
(SEQ ID NO: 31)
TYFANNFD
131R010 Alternative Heavy chain CDR3
(SEQ ID NO: 32)
ATYFANNFDY
131R010 Light chain CDR1
(SEQ ID NO: 33)
KASQSVDYDGDSYMN
131R010 Light chain CDR2
(SEQ ID NO: 34)
AASNLES
131R010 Alternative Light chain CDR2
(SEQ ID NO: 35)
AAS
131R010 Light chain CDR3
(SEQ ID NO: 36)
QQSNEDPLT
131R010 Alternative Light chain CDR3
(SEQ ID NO: 37)
QQSNEDPLTF
131R010 Heavy chain variable region amino acid sequence
(SEQ ID NO: 3 8)
QVQLVQSGAEVKKPGASVKVSCKASGYTFTDYSIHWVRQAPGQGLEWIGYTYPSNGDSGY
NQKFKNRVTMTRDTSTSTAYMELSRLRSEDTAVYYCATYFANNFDYWGQGTTLTVSS
131R010 Light chain variable region amino acid sequence
(SEQ ID NO: 39)
DIQMTQSPSSLSASVGDRVTITCKASQSVDYDGDSYMNWYQQKPGKAPKLLIYAASNLES
GVPSRFSGSGSGTDFTLTISPVQAEDFATYYCQQSNEDPLTFGAGTKLELKR
131R010 Heavy chain amino acid sequence with predicted signal
sequence underlined
(SEQ ID NO: 40)
MKHLWFFLLLVAAPRWVLSQVQLVQSGAEVKKPGASVKVSCKASGYTFTDYSIHWVRQAP
GQGLEWIGYTYPSNGDSGYNQKFKNRVTMTRDTSTSTAYMELSRLRSEDTAVYYCATYFA
NNFDYWGQGTTLTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKRVEPKSCD
KTHTCPPCPAPELLGGPSVFLEPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
131R010 Heavy chain amino acid sequence without predicted signal
sequence
(SEQ ID NO: 41)
QVQLVQSGAEVKKPGASVKVSCKASGYTFTDYSIHWVRQAPGQGLEWIGYTYPSNGDSGY
NQKFKNRVTMTRDTSTSTAYMELSRLRSEDTAVYYCATYFANNFDYWGQGTTLTVSSAST
KGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQG
NVFSCSVMHEALHNHYTQKSLSLSPGK
131R010 Light chain amino acid sequence with predicted signal
sequence underlined
(SEQ ID NO: 42)
MKHLWEELLLVAAPRWVLSDIQMTQSPSSLSASVGDRVTITCKASQSVDYDGDSYMNWYQ
QKPGKAPKLLIYAASNLESGVPSRFSGSGSGTDFTLTISPVQAEDFATYYCQQSNEDPLT
FGAGTKLELKRTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTKSFNRGEC
131R010 Light chain amino acid sequence without predicted signal
sequence
(SEQ ID NO: 43)
DIQMTQSPSSLSASVGDRVTITCKASQSVDYDGDSYMNWYQQKPGKAPKLLIYAASNLES
GVPSRFSGSGSGTDFTLTISPVQAEDFATYYCQQSNEDPLTFGAGTKLELKRTVAAPSVF
IFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC
h89M5-H2L2 Heavy chain variable region amino acid sequence
(SEQ ID NO: 44)
QVQLVQSGAEVKKPGASVKVSCKTSGYTFTGYTMHWVRQAPGQRLEWMGGINPNNGGTTY
NQNFKGRVTITRDTSASTAYMELSSLRSEDTAVYYCARKEFSDGYYFFAYWGQGTLVTVS
S
h89M5-H2L2 Light chain variable region amino acid sequence
(SEQ ID NO: 45)
DIQMTQSPSSLSASVGDRVTITCKASQDVIFAVAWYQQKPGKAPKLLIYWASTRHTGVPS
RFSGSGSGTDYTLTISSLQPEDFATYYCQQHYSTPWTFGGGTKVEIK
h89M5-H2L2 Heavy chain amino acid sequence with predicted signal
sequence underlined
(SEQ ID NO: 46)
MDWTWRILFLVAAATGAHSQVQLVQSGAEVKKPGASVKVSCKTSGYTFTGYTMHWVRQAP
GQRLEWMGGINPNNGGTTYNQNFKGRVTITRDTSASTAYMELSSLRSEDTAVYYCARKEF
SDGYYFFAYWGQGTLVTVSSASTKGPSVFPLAPCSRSTSESTAALGCLVKDYFPEPVTVS
WNSGALTSGVHTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPAPIEKTISKTKG
QPREPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK
h89M5-H2L2 Heavy chain amino acid sequence without predicted/
signal sequence
(SEQ ID NO: 47)
QVQLVQSGAEVKKPGASVKVSCKTSGYTFTGYTMHWVRQAPGQRLEWMGGINPNNGGTTY
NQNFKGRVTITRDTSASTAYMELSSLRSEDTAVYYCARKEFSDGYYFFAYWGQGTLVTVS
SASTKGPSVFPLAPCSRSTSESTAALGCLVKDYFPEPVTVSWNSGALTSGVHTFPAVLQS
SGLYSLSSVVTVPSSNFGTQTYTCNVDHKPSNTKVDKTVERKCCVECPPCPAPPVAGPSV
FLFPPKPKDTLMISRTPEVTCVVVDVSHEDPEVQFNWYVDGVEVHNAKTKPREEQFNSTF
RVVSVLTVVHQDWLNGKEYKCKVSNKGLPAPIEKTISKTKGQPREPQVYTLPPSREEMTK
NQVSLTCLVKGFYPSDIAVEWESNGQPENNYKTTPPMLDSDGSFFLYSKLTVDKSRWQQG
NVFSCSVMHEALHNHYTQKSLSLSPGK
h89M5-H2L2 Light chain amino acid sequence with predicted signal
sequence underlined
(SEQ ID NO: 48)
MDMRVPAQLLGLLLLWLRGARCDIQMTQSPSSLSASVGDRVTITCKASQDVIFAVAWYQQ
KPGKAPKLLIYWASTRHTGVPSRFSGSGSGTDYTLTISSLQPEDFATYYCQQHYSTPWTF
GGGTKVEIKRTVAAPSVFIFPPSDEQLKSGTASVVCLLNNFYPREAKVQWKVDNALQSGN
SQESVTEQDSKDSTYSLSNTLTLSKADYEKHKVYACEVTHQGLSSPVTKSFNRGEC
h89M5-H2L2 Light chain amino acid sequence without predicted
signal sequence
(SEQ ID NO: 49)
DIQMTQSPSSLSASVGDRVTITCKASQDVIFAVAWYQQKPGKAPKLLIYWASTRHTGVPS
RFSGSGSGTDYTLTISSLQPEDFATYYCQQHYSTPWTFGGGTKVEIKRTVAAPSVFIFPP
SDEQLKSGTASVVCLLNNFYPREAKVQWKVDNALQSGNSQESVTEQDSKDSTYSLSNTLT
LSKADYEKHKVYACEVTHQGLSSPVTKSFNRGEC
h130M23-H1L2 Light chain variable region amino acid sequence
(SEQ ID NO: 50)
DIQMTQSPSSLSASVGDRVTITCKASQDVSSAVAWYQQKPGKAPKLLIYWASTRHTGVPS
RFSGSGSGTDFTLTISSVQAEDFATYYCQQHYSTPWTFGQGTKVEIK
h130M23-H1L2 Light chain amino acid sequence with predicted
signal sequence underlined
(SEQ ID NO: 51)
MKYLLPTAAAGLLLLAAQPAMADIQMTQSPSSLSASVGDRVTITCKASQDVSSAVAWYQQ
KPGKAPKLLIYWASTRHTGVPSRFSGSGSGTDFTLTISSVQAEDFATYYCQQHYSTPWTF
GQGTKVEIKRTVAAPSVFIFPPSDEQLKSGTASVVCLLNNFYPREAKVQWKVDNALQSGN
SQESVTEQDSKDSTYSLSNTLTLSKADYEKHKVYACEVTHQGLSSPVTKSFNRGEC
h130M23-H1L2 Light chain amino acid sequence without predicted
signal sequence
(SEQ ID NO: 52)
DIQMTQSPSSLSASVGDRVTITCKASQDVSSAVAWYQQKPGKAPKLLIYWASTRHTGVPS
RFSGSGSGTDFTLTISSVQAEDFATYYCQQHYSTPWTFGQGTKVEIKRTVAAPSVFIFPP
SDEQLKSGTASVVCLLNNFYPREAKVQWKVDNALQSGNSQESVTEQDSKDSTYSLSNTLT
LSKADYEKHKVYACEVTHQGLSSPVTKSFNRGEC
Human LGR4 protein sequence
(NM_018490; SEQ ID NO: 53)
MPGPLGLLCFLALGLLGSAGPSGAAPPLCAAPCSCDGDRRVDCSGKGLTAVPEGLSAFTQ
ALDISMNNITQLPEDAFKNFPFLEELQLAGNDLSFIHPKALSGLKELKVLTLQNNQLKTV
PSEAIRGLSALQSLRLDANHITSVPEDSFEGLVQLRHLWLDDNSLTEVPVHPLSNLPTLQ
ALTLALNKISSIPDFAFTNLSSLVVLHLHNNKIRSLSQHCFDGLDNLETLDLNYNNLGEF
PQAIKALPSLKELGFHSNSISVIPDGAFDGNPLLRTIHLYDNPLSFVGNSAFHNLSDLHS
LVIRGASMVQQFPNLTGTVHLESLTLTGTKISSIPNNLCQEQKMLRTLDLSYNNIRDLPS
FNGCHALEEISLQRNQIYQIKEGTFQGLISLRILDLSRNLIHEIHSRAFATLGPITNLDV
SFNELTSFPTEGLNGLNQLKLVGNFKLKEALAAKDFVNLRSLSVPYAYQCCAFWGCDSYA
NLNTEDNSLQDHSVAQEKGTADAANVTSTLENEEHSQIIIHCTPSTGAFKPCEYLLGSWM
IRLTVWFIFLVALFFNLLVILTTFASCTSLPSSKLFIGLISVSNLFMGIYTGILTFLDAV
SWGRFAEFGIWWETGSGCKVAGFLAVFSSESAIFLLMLATVERSLSAKDIMKNGKSNHLK
QFRVAALLAFLGATVAGCFPLFHRGEYSASPLCLPFPTGETPSLGFTVTLVLLNSLAFLL
MAVIYTKLYCNLEKEDLSENSQSSMIKHVAWLIFTNCIFFCPVAFFSFAPLITAISISPE
IMKSVTLIFFPLPACLNPVLYVFFNPKFKEDWKLLKRRVTKKSGSVSVSISSQGGCLEQD
FYYDCGMYSHLQGNLTVCDCCESFLLTKPVSCKHLIKSHSCPALAVASCQRPEGYWSDCG
TQSAHSDYADEEDSFVSDSSDQVQACGRACFYQSRGFPLVRYAYNLPRVKD
Human LGR5 protein sequence
(SEQ ID NO: 54)
MDTSRLGVLLSLPVLLQLATGGSSPRSGVLLRGCPTHCHCEPDGRMLLRVDCSDLGLSEL
PSNLSVFTSYLDLSMNNISQLLPNPLPSLRFLEELRLAGNALTYIPKGAFTGLYSLKVLM
LQNNQLRHVPTEALQNLRSLQSLRLDANHISYVPPSCFSGLHSLRHLWLDDNALTEIPVQ
AFRSLSALQAMTLALNKIHHIPDYAFGNLSSLVVLHLHNNRIHSLGKKCFDGLHSLETLD
LNYNNLDEFPTAIRTLSNLKELGFHSNNIRSIPEKAFVGNPSLITIHFYDNPIQFVGRSA
FQHLPELRTLTLNGASQITEFPDLTGTANLESLTLTGAQISSLPQTVCNQLPNLQVLDLS
YNLLEDLPSFSVCQKLQKIDLRHNEIYEIKVDTFQQLLSLRSLNLAWNKIAIIHPNAFST
LPSLIKLDLSSNLLSSFPITGLHGLTHLKLTGNHALQSLISSENFPELKVIEMPYAYQCC
AFGVCENAYKISNQWNKGDNSSMDDLHKKDAGMFQAQDERDLEDFLLDFEEDLKALHSVQ
CSPSPGPFKPCEHLLDGWLIRIGVWTIAVLALTCNALVTSTVFRSPLYISPIKLLIGVIA
AVNMLTGVSSAVLAGVDAFTFGSFARHGAWWENGVGCHVIGFLSIFASESSVFLLTLAAL
ERGFSVKYSAKFETKAPFSSLKVIILLCALLALTMAAVPLLGGSKYGASPLCLPLPFGEP
STMGYMVALILLNSLCFLMMTIAYTKLYCNLDKGDLENIWDCSMVKHIALLLFTNCILNC
PVAFLSFSSLINLTFISPEVIKFILLVVVPLPACLNPLLYILFNPHFKEDLVSLRKQTYV
WTRSKHPSLMSINSDDVEKQSCDSTQALVTFTSSSITYDLPPSSVPSPAYPVTESCHLSS
VAFVPCL
Human LGR6 protein sequence
(BC047905; SEQ ID NO: 55)
MGRPRLTLVCQVSIIISARDLSMNNLTELQPGLFHHLRFLEELRLSGNHLSHIPGQAFSG
LYSLKILMLQNNQLGGIPAEALWELPSLQSLRLDANLISLVPERSFEGLSSLRHLWLDDN
ALTEIPVRALNNLPALQAMTLALNRISHIPDYAFQNLTSLVVLHLHNNRIQHLGTHSFEG
LHNLETLDLNYNKLQEFPVAIRTLGRLQELGFHNNNIKAIPEKAFMGNPLLQTIHFYDNP
IQFVGRSAFQYLPKLHTLSLNGAMDIQEFPDLKGTTSLEILTLTRAGIRLLPSGMCQQLP
RLRVLELSHNQIEELPSLHRCQKLEEIGLQHNRIWEIGADTFSQLSSLQALDLSWNAIRS
IHPEAFSTLHSLVKLDLTDNQLTTLPLAGLGGLMHLKLKGNLALSQAFSKDSFPKLRILE
VPYAYQCCPYGMCASFFKASGQWEAEDLHLDDEESSKRPLGLLARQAENHYDQDLDELQL
EMEDSKPHPSVQCSPTPGPFKPCEYLFESWGIRLAVWAIVLLSVLCNGLVLLTVFAGGPV
PLPPVKFVVGAIAGANTLTGISCGLLASVDALTFGQFSEYGARWETGLGCRATGFLAVLG
SEASVLLLTLAAVQCSVSVSCVRAYGKSPSLGSVRAGVLGCLALAGLAAALPLASVGEYG
ASPLCLPYAPPEGQPAALGFTVALVMMNSFCFLVVAGAYIKLYCDLPRGDFEAVWDCAMV
RHVAWLIFADGLLYCPVAFLSFASMLGLFPVTPEAVKSVLLVVLPLPACLNPLLYLLFNP
HFRDDLRRLRPRAGDSGPLAYAAAGELEKSSCDSTQALVAFSDVDLILEASEAGRPPGLE
TYGFPSVTLISCQQPGAPRLEGSHCVEPEGNHFGNPQPSMDGELLLRAEGSTPAGGGLSG
GGGFQPSGLAFASHV
LGR5 ECD amino acids 22-564
(SEQ ID NO: 56)
GSSPRSGVLLRGCPTHCHCEPDGRMLLRVDCSDLGLSELPSNLSVFTSYLDLSMNNISQL
LPNPLPSLRFLEELRLAGNALTY IPKGAFTGLYSLKVLMLQNNQLRHVPTEALQNLRSLQ
SLRLDANHISYVPPSCFSGLHSLRHLWLDDNALTEIPVQAFRSLSALQAMTLALNKIHHI
PDYAFGNLSSLVVLHLHNNRIHSLGKKCFDGLHSLETLDLNYNNLDEFPTAIRTLSNLKE
LGFHSNNIRSIPEKAFVGNPSLITIHFYDNPIQFVGRSAFQHLPELRTLTLNGASQITEF
PDLTGTANLESLTLTGAQISSLPQTVCNQLPNLQVLDLSYNLLEDLPSFSVCQKLQKIDL
RHNEIYEIKVDTFQQLLSLRSLNLAWNKIAIIHPNAFSTLPSLIKLDLSSNLLSSFPITG
LHGLTHLKLTGNHALQSLISSENFPELKVIEMPYAYQCCAFGVCENAYKISNQWNKGDNS
SMDDLHKKDAGMFQAQDERDLEDFLLDFEEDLKALHSVQCSPSPGPFKPCEHLLDGWLIR
IGV
LGR5-Fc protein sequence
(SEQ ID NO: 57)
MDTSRLGVLLSLPVLLQLATGGSSPRSGVLLRGCPTHCHCEPDGRMLLRVDCSDLGLSEL
PSNLSVFTSYLDLSMNNISQLLPNPLPSLRFLEELRLAGNALTYIPKGAFTGLYSLKVLM
LQNNQLRHVPTEALQNLRSLQSLRLDANHISYVPPSCFSGLHSLRHLWLDDNALTEIPVQ
AFRSLSALQAMTLALNKIHHIPDYAFGNLSSLVVLHLHNNRIHSLGKKCFDGLHSLETLD
LNYNNLDEFPTAIRTLSNLKELGFHSNNIRSIPEKAFVGNPSLITIHFYDNPIQFVGRSA
FQHLPELRTLTLNGASQITEFPDLTGTANLESLTLTGAQISSLPQTVCNQLPNLQVLDLS
YNLLEDLPSFSVCQKLQKIDLRHNEIYEIKVDTFQQLLSLRSLNLAWNKIAIIHPNAFST
LPSLIKLDLSSNLLSSFPITGLHGLTHLKLTGNHALQSLISSENFPELKVIEMPYAYQCC
AFGVCENAYKISNQWNKGDNSSMDDLHKKDAGMFQAQDERDLEDFLLDFEEDLKALHSVQ
CSPSPGPFKPCEHLLDGWLIRIGVGRADKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALH
NHYTQKSLSLSPGK
Human IgG₁ Fc region
(SEQ ID NO: 58)
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
Human IgG₁ Fc region
(SEQ ID NO: 59)
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK
Human IgG₁ Fc region
(SEQ ID NO: 60)
KSSDKTHTCPPCPAPELLGGPSVFLEPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
Human IgG₁ Fc region
(SEQ ID NO: 61)
EPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
Human IgG₂ Fc region
(SEQ ID NO: 62)
CVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDSDGS
FFLYSKLTVDKSRWQQGNVFSCSVMHEALHNHYTQKSLSLSPGK
LGR5-Fc protein sequence without predicted signal sequence
(SEQ ID NO: 63)
GSSPRSGVLLRGCPTHCHCEPDGRMLLRVDCSDLGLSELPSNLSVFTSYLDLSMNNISQL
LPNPLPSLRFLEELRLAGNALTYIPKGAFTGLYSLKVLMLQNNQLRHVPTEALQNLRSLQ
SLRLDANHISYVPPSCFSGLHSLRHLWLDDNALTEIPVQAFRSLSALQAMTLALNKIHHI
PDYAFGNLSSLVVLHLHNNRIHSLGKKCFDGLHSLETLDLNYNNLDEFPTAIRTLSNLKE
LGFHSNNIRSIPEKAFVGNPSLITIHFYDNPIQFVGRSAFQHLPELRTLTLNGASQITEF
PDLTGTANLESLTLTGAQISSLPQTVCNQLPNLQVLDLSYNLLEDLPSFSVCQKLQKIDL
RHNEIYEIKVDTFQQLLSLRSLNLAWNKIAIIHPNAFSTLPSLIKLDLSSNLLSSFPITG
LHGLTHLKLTGNHALQSLISSENFPELKVIEMPYAYQCCAFGVCENAYKISNQWNKGDNS
SMDDLHKKDAGMFQAQDERDLEDFLLDFEEDLKALHSVQCSPSPGPFKPCEHLLDGWLIR
IGVGRADKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK

What is claimed is:

1. A method of treating cancer comprising administering to a subject a therapeutically effective amount of a RSPO-LGR pathway inhibitor and a therapeutically effective amount of a mitotic inhibitor, wherein the RSPO-LGR inhibitor and the mitotic inhibitor are administered using a staggered dosing schedule and the RSPO-LGR inhibitor is administered first; and wherein the RSPO-LGR inhibitor is:
(a) an antibody that specifically binds at least one human RSPO protein,
(b) an antibody that specifically binds at least one human LGR protein, or
(c) a soluble receptor comprising an extracellular domain of a human LGR protein capable of binding at least one human RSPO protein.

2. A method of treating cancer comprising administering to a subject a therapeutically effective amount of an antibody that specifically binds at least one human RSPO protein and a therapeutically effective amount of a mitotic inhibitor, wherein the antibody and the mitotic inhibitor are administered using a staggered dosing schedule and the antibody is administered first.

3. A method of treating cancer comprising administering to a subject a therapeutically effective amount of an antibody that specifically binds human RSPO3 and a therapeutically effective amount of a mitotic inhibitor, wherein the antibody and the mitotic inhibitor are administered using a staggered dosing schedule and the antibody is administered first.

4. The method of any one of claims 1 to 3, wherein the mitotic inhibitor is administered about 1, 2, 3, 4, 5, or 6 days after the RSPO-LGR pathway inhibitor or antibody is administered.

5. A method of treating cancer in a subject comprising administering to the subject a therapeutically effective amount of a RSPO-LGR pathway inhibitor, wherein the RSPO-LGR pathway inhibitor is:
(a) an antibody that specifically binds at least one human RSPO protein,
(b) an antibody that specifically binds at least one human LGR protein, or
(c) a soluble receptor comprising an extracellular domain of a human LGR protein capable of binding at least one human RSPO protein, and
wherein the subject is scheduled to receive a therapeutically effective amount of a mitotic inhibitor about 1, 2, 3, 4, 5, or 6 days after the administration of the RSPO-LGR pathway inhibitor.

6. A method of treating cancer in a subject comprising administering to the subject a therapeutically effective amount of an antibody that specifically binds at least one human RSPO protein, wherein the subject is scheduled to receive a therapeutically effective amount of a mitotic inhibitor about 1, 2, 3, 4, 5, or 6 days after the administration of the antibody.

7. A method of treating cancer in a subject comprising administering to the subject a therapeutically effective amount of an antibody that specifically binds human RSPO3, wherein the subject is scheduled to receive a therapeutically effective amount of a mitotic inhibitor about 1, 2, 3, 4, 5, or 6 days after the administration of the antibody.

8. A method of increasing the efficacy of a mitotic inhibitor in treating cancer in a subject comprising administering to the subject a mitotic inhibitor about 1, 2, 3, 4, 5, or 6 days after a RSPO-LGR pathway inhibitor has been administered, wherein the RSPO-LGR pathway inhibitor is:
(a) an antibody that specifically binds at least one human RSPO protein,
(b) an antibody that specifically binds at least one human LGR protein, or
(c) a soluble receptor comprising an extracellular domain of a human LGR protein capable of binding at least one human RSPO protein.

9. A method of increasing the efficacy of a mitotic inhibitor in treating cancer in a subject comprising administering to the subject a mitotic inhibitor about 1, 2, 3, 4, 5, or 6 days after an antibody that specifically binds at least one human RSPO protein has been administered.

10. A method of increasing the efficacy of a mitotic inhibitor in treating cancer in a subject comprising administering to the subject a mitotic inhibitor about 1, 2, 3, 4, 5, or 6 days after an antibody that specifically binds human RSPO3 has been administered.

11. A method of increasing the efficacy of a mitotic inhibitor in treating cancer in a subject comprising:
(a) administering to the subject a RSPO-LGR pathway inhibitor; and
(b) administering to the subject a mitotic inhibitor about 1, 2, 3, 4, 5, or 6 days after the RSPO-LGR pathway inhibitor has been administered,
wherein the RSPO-LGR pathway inhibitor is:
(i) an antibody that specifically binds at least one human RSPO protein,
(ii) an antibody that specifically binds at least one human LGR protein, or
(iii) a soluble receptor comprising an extracellular domain of a human LGR protein capable of binding at least one human RSPO protein.

12. A method of increasing the efficacy of a mitotic inhibitor in treating cancer in a subject comprising:
(a) administering to the subject an antibody that specifically binds at least one human RSPO protein; and
(b) administering to the subject a mitotic inhibitor about 1, 2, 3, 4, 5, or 6 days after the antibody has been administered.

13. A method of increasing the efficacy of a mitotic inhibitor in treating cancer in a subject comprising:
(a) administering to the subject an antibody that specifically binds human RSPO3; and
(b) administering to the subject a mitotic inhibitor about 1, 2, 3, 4, 5, or 6 days after the antibody has been administered.

14. The method of any one of claims 1 to 13, wherein the mitotic inhibitor is administered about 1 day after administration of the RSPO-LGR pathway inhibitor or antibody.

15. The method of any one of claims 1 to 13, wherein the mitotic inhibitor is administered about 2 days after administration of the RSPO-LGR pathway inhibitor or antibody.

16. The method of any one of claims 1 to 13, wherein the mitotic inhibitor is administered about 3 days after administration of the RSPO-LGR pathway inhibitor or antibody.

17. The method of any one of claims 1 to 16, wherein the RSPO-LGR pathway inhibitor or antibody and the mitotic inhibitor act synergistically.

18. The method of any one of claims 1 to 17, wherein the RSPO-LGR pathway inhibitor or antibody is administered about once a week, about once every two weeks, or about once every 3 weeks.

19. The method of any one of claims 1 to 17, wherein the RSPO-LGR pathway inhibitor or antibody is administered about once every 3 weeks and the mitotic inhibitor is administered about once a week.

20. The method of any one of claims 1 to 17, wherein the RSPO-LGR pathway inhibitor or antibody is administered about once every 4 weeks.

21. The method of any one of claims 1 to 18 or 20, wherein the mitotic inhibitor is administered about once a week, about once every 2 weeks, about once every 3 weeks, or about once a week for 3 weeks of a 4 week cycle.

22. The method of any one of claims 1 to 17, wherein the RSPO-LGR pathway inhibitor or antibody is administered about once every 4 weeks and the mitotic inhibitor is administered about once a week.

23. The method of any one of claims 1 to 22, wherein the RSPO-LGR pathway inhibitor or antibody is administered for 2, 3, 4, 5, 6, 7, 8, or more cycles.

24. The method of any one of claims 1 to 23, wherein the mitotic inhibitor is administered for 2, 3, 4, 5, 6, 7, 8, or more cycles.

25. The method of any one of claims 1 to 24, wherein the RSPO-LGR pathway inhibitor or antibody is administered to the subject at a dosage of about 2 mg/kg to about 20 mg/kg.

26. The method of claim 25, wherein the RSPO-LGR pathway inhibitor or antibody is administered at a dosage of about 2.5 mg/kg to about 20 mg/kg every two weeks.

27. The method of claim 25, wherein the RSPO-LGR pathway inhibitor or antibody is administered at a dosage of about 5 mg/kg to about 20 mg/kg every three weeks.

28. The method of any one of claims 1 to 27, wherein the RSPO-LGR pathway inhibitor is an antibody that specifically binds at least one human RSPO protein.

29. The method of claim 28, wherein the antibody specifically binds at least one human RSPO protein selected from the group consisting of: RSPO1, RSPO2, and RSPO3.

30. The method of claim 28, wherein the antibody specifically binds at least human RSPO1.

31. The method of claim 28, wherein the antibody comprises:
(a) a heavy chain CDR1 comprising TGYTMH (SEQ ID NO:5), a heavy chain CDR2 comprising GINPNNGGTTYNQNFKG (SEQ ID NO:6), and a heavy chain CDR3 comprising KEFSDGYYFFAY (SEQ ID NO:7); and
(b) a light chain CDR1 comprising KASQDVIFAVA (SEQ ID NO:8), a light chain CDR2 comprising WASTRHT (SEQ ID NO:9), and a light chain CDR3 comprising QQHYSTPW (SEQ ID NO:10).

32. The method of claim 28, wherein the antibody comprises a heavy chain variable region comprising SEQ ID NO:11 or SEQ ID NO:44 and a light chain variable region comprising SEQ ID NO:12 or SEQ ID NO:45.

33. The method of claim 28, wherein the antibody comprises a heavy chain variable region comprising SEQ ID NO:11 and a light chain variable region comprising SEQ ID NO:12.

34. The method of claim 28, wherein the antibody specifically binds at least human RSPO2.

35. The method of claim 28, wherein the antibody comprises:
(a) a heavy chain CDR1 comprising SSYAMS (SEQ ID NO:17), a heavy chain CDR2 comprising SISSGGSTYYPDSVKG (SEQ ID NO:18), and a heavy chain CDR3 comprising RGGDPGVYNGDYEDAMDY (SEQ ID NO:19); and
(b) a light chain CDR1 comprising KASQDVSSAVA (SEQ ID NO:20), a light chain CDR2 comprising WASTRHT (SEQ ID NO:21), and a light chain CDR3 comprising QQHYSTP (SEQ ID NO:22).

36. The method of claim 28, wherein the antibody comprises a heavy chain variable region comprising SEQ ID NO:23 and a light chain variable region comprising SEQ ID NO:24.

37. The method of claim 28, wherein the antibody specifically binds at least human RSPO3.

38. The method of claim 28, wherein the antibody comprises:
(a) a heavy chain CDR1 comprising DYSIH (SEQ ID NO:29), a heavy chain CDR2 comprising YIYPSNGDSGYNQKFK (SEQ ID NO:30), and a heavy chain CDR3 comprising TYFANNFD (SEQ ID NO:31) or ATYFANNTDY(SEQ ID NO:32); and
(b) a light chain CDR1 comprising KASQSVDYDGDSYMN (SEQ ID NO:33), a light chain CDR2 comprising AASNLES (SEQ ID NO:34) or AAS (SEQ ID NO:35), and a light chain CDR3 comprising QQSNEDPLT (SEQ ID NO:36) or QQSNEDPLTF (SEQ ID NO:37).

39. The method of claim 38, wherein the antibody comprises:
(a) a heavy chain CDR1 comprising DYSIH (SEQ ID NO:29), a heavy chain CDR2 comprising YIYPSNGDSGYNQKFK (SEQ ID NO:30), and a heavy chain CDR3 comprising TYFANNFD (SEQ ID NO:32); and
(b) a light chain CDR1 comprising KASQSVDYDGDSYMN (SEQ ID NO:33), a light chain CDR2 comprising AASNLES (SEQ ID NO:34), and a light chain CDR3 comprising QQSNEDPLT (SEQ ID NO:36).

40. The method of claim 28, wherein the antibody comprises a heavy chain variable region comprising SEQ ID NO:38 and a light chain variable region comprising SEQ ID NO:39.

41. The method of any one of claims 1 to 27, wherein the RSPO-LGR pathway inhibitor is an antibody that specifically binds at least one human LGR protein.

42. The method of claim 41, wherein the antibody specifically binds at least one human LGR protein selected from the group consisting of: LGR4, LGR5, and LGR6.

43. The method of claim 41, wherein the antibody specifically binds at least human LGR5.

44. The method of claim 41, wherein the antibody comprises:
(a) the heavy chain CDR1, CDR2, and CDR3 sequences of the monoclonal antibody produced by the 88M1 hybridoma having the ATCC deposit number PTA-9342; and
(b) the light chain CDR1, CDR2, and CDR3 sequences of the monoclonal antibody produced by the 88M1 hybridoma having the ATCC deposit number PTA-9342.

45. The method of claim 41, wherein the antibody comprises the heavy chain variable region and light chain variable region of the monoclonal antibody produced by the 88M1 hybridoma having the ATCC deposit number PTA-9342.

46. The method of any one of claims 1 to 45, wherein the antibody is a monoclonal antibody, a recombinant antibody, a chimeric antibody, a humanized antibody, a human antibody, or an antibody fragment comprising an antigen-binding site.

47. The method of any one of claims 1 to 46, wherein the antibody is a monospecific antibody or a bispecific antibody.

48. The method of any one of claims 1 to 39, wherein the antibody is an IgG1 antibody or an IgG2 antibody.

49. The method of any one of claims 1 to 30, 37 to 40, or 46 to 48, wherein the RSPO-LGR pathway inhibitor is OMP-131R010.

50. The method of any one of claims 1 to 27, wherein the RSPO-LGR pathway inhibitor is a soluble receptor comprising an extracellular domain of a human LGR protein, wherein the extracellular domain is capable of binding a human RSPO protein.

51. The method of claim 50, wherein the human LGR protein is LGR5.

52. The method of claim 50, wherein the extracellular domain of a human LGR protein comprises amino acids 22-564 of human LGR5 (SEQ ID NO:56).

53. The method of any one of claims 50 to 52, wherein the soluble receptor comprises a non-LGR polypeptide.

54. The method of claim 53, wherein the non-LGR polypeptide is directly linked to the extracellular domain of the human LGR protein.

55. The method of claim 53, wherein the non-LGR polypeptide is connected to the extracellular domain of the human LGR protein by a linker.

56. The method of any one of claims 53 to 55, wherein the non-LGR polypeptide comprises a human Fc region.

57. The method of any one of claims 50 to 56, wherein the non-LGR polypeptide comprises SEQ ID NO:58, SEQ ID NO:59, SEQ ID NO:60, SEQ ID NO:61, or SEQ ID NO:62.

58. The method of any one of claims 1 to 57, wherein the mitotic inhibitor is a taxane, a vinca alkaloid, an epothilone, or eribulin mesylate.

59. The method of claim 58, wherein the mitotic inhibitor is a taxane selected from the group consisting of paclitaxel, docetaxel, and derivatives thereof.

60. The method of claim 59, wherein the mitotic inhibitor is paclitaxel or nab-paclitaxel.

61. The method of claim 59, wherein the mitotic inhibitor is docetaxel.

62. The method of claim 58, wherein the mitotic inhibitor is a vinca alkaloid selected from the group consisting of vinblastine, vincristine, vinorelbine, and derivatives thereof.

63. The method of any one of claims 1 to 62, wherein the cancer is colorectal cancer, breast cancer, ovarian cancer, lung cancer, or pancreatic cancer.

64. The method of claim 63, wherein the cancer is colorectal cancer.

65. The method of any one of claims 1 to 64, which further comprises administering at least one additional therapeutic agent.

66. The method of claim 65, wherein the additional therapeutic agent is a chemotherapeutic agent.

67. A method of treating cancer comprising administering to a subject a therapeutically effective amount of OMP-131R010 and a therapeutically effective amount of a taxane selected from the group consisting of paclitaxel, nab-paclitaxel, and docetaxel, wherein the taxane is administered about 1, 2, 3, 4, 5, or 6 days after OMP-131R010 is administered.

68. The method of claim 67, wherein OMP-131R010 is administered about once every 3 weeks.

69. The method of claim 67, wherein OMP-131R010 is administered about once every 2 weeks.

70. The method of any one of claims 67 to 69, wherein the taxane is administered about once a week.

71. The method of any one of claims 67 to 69, wherein the taxane is administered about once every two weeks.

72. The method of any one of claims 67 to 69, wherein the taxane is administered about once every three weeks.

73. The method of any one of claims 67 to 72, further comprising the administration of an additional therapeutic agent.

74. The method of claim 73, wherein the additional therapeutic agent is a chemotherapeutic agent.

75. The method of any one of claims 67 to 74, wherein the cancer is colorectal cancer, breast cancer, ovarian cancer, lung cancer, or pancreatic cancer.

76. The method of claim 75, wherein the cancer is colorectal cancer.

77. The method of claim 64 or claim 76, wherein the colorectal cancer comprises an inactivating mutation in the adenomatous polyposis coli (APC) gene.

78. The method of claim 64 or claim 76, wherein the colorectal cancer does not comprise an inactivating mutation in the APC gene.

79. The method of claim 64 or claim 76, wherein the colorectal cancer comprises a wild-type APC gene.

80. The method of claim 64 or claim 76, wherein the colorectal cancer does not comprise an activating mutation in the β-catenin gene.

81. The method of claim 64 or claim 76, wherein the colorectal cancer comprises a RSPO gene fusion.

82. The method of claim 81, wherein the RSPO gene fusion is a RSPO2 gene fusion.

83. The method of claim 81, wherein the RSPO gene fusion is a RSPO3 gene fusion.

84. The method of any one of claims 1 to 83, further comprising determining if the cancer has an inactivating mutation in the APC gene.

85. The method of any one of claims 1 to 84, further comprising determining if the tumor or cancer has an activating mutation in the β-catenin gene.

86. The method of any one of claims 1 to 85, further comprising determining if the tumor or cancer has a RSPO gene fusion.

87. The method of claim 86, wherein the RSPO gene fusion is a RSPO2 gene fusion.

88. The method of claim 86, wherein the RSPO gene fusion is a RSPO3 gene fusion.

89. The method of any one of claims 86 to 88, wherein the RSPO gene fusion is determined by a PCR-based assay, microarray analysis, or nucleotide sequencing.

90. The method of any one of claims 1 to 89, wherein the cancer expresses high RSPO1, RSPO2, RSPO3, and/or RSPO4 levels compared to a pre-determined level of expression of RSPO1, RSPO2, RSPO3, and/or RSPO4, respectively.

91. The method of claim 90, wherein the pre-determined RSPO1, RSPO2, RSPO3, or RSPO4 expression level is the level of RSPO1, RSPO2, RSPO3, or RSPO4 expression in a tumor or a group of tumors of the same tissue type.

92. The method according to any one of claims 1 to 91, further comprising determining the expression level of one or more of RSPO1, RSPO2, RSPO3, and RSPO4 in the cancer.

93. The method according to claim 92, wherein the expression level of one or more of RSPO1, RSPO2, RSPO3, and RSPO4 is determined by a PCR-based assay, microarray analysis, or nucleotide sequencing.
